(19) United States
(12) Patent Application Publication
(10) Pub. No.: US 2007/0225269 A1
(43) Pub. Date: Sep. 27, 2007

(54) Use of (3-(1-(3-Phenyl-Propenyl)-Piperidin-4-Yl)-2,3-Dihydro-Indol-1-Yl)-(Pyridin-4-Yl)-Methanonone Derivatives and Related Compounds as Insecticides

(76) Inventors: Jerome Cassayre, Basel (CH); Louis-Pierre Molleyres, Basel (CH); Peter Maienfisch, Basel (CH); Fredrik Cederbaum, Basel (CH)

Correspondence Address: SYNGENTA CROP PROTECTION , INC.;PATENT AND TRADEMARK DEPARTMENT, 410 SWING ROAD, GREENSBORO, NC, 27409, US

(21) Appl. No.: 10/581,173

(22) PCT Filed: Dec. 9, 2004
(86) PCT No.: PCT/IB04/04170

§ 371 Date: Jan. 23, 2007

(30) Foreign Application Priority Data
Dec. 12, 2003 (GB) 0328909.7

(51) Int. Cl. A01N 43/38; C07D 401/14

(52) U.S. Cl. 514/210.21; 514/336; 514/414

(57) ABSTRACT

The use of a compound of formula (I): R³ N Y^{R1}(I) wherein Y is a single bond, C═O, C═S or S(O)₇₁ where m is 0, 1 or 2; R¹, R², R³, R⁴, R⁸ and Ra are specified organic groups and p is 0, 1, 2, 3, 4, 5 or 6; q is 0, 1, 2, 3, 4, 5 or 6; p+q is 1, 2, 3,4, 5 or 6; or salts or N-oxides thereof or compositions to containing them in controlling insects, acarines, nematodes or molluscs; novel compounds are also provided.

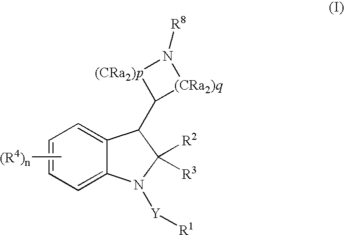

The present invention relates to indoline derivatives, to processes for preparing them, to insecticidal, acaricidal, molluscicidal and nematicidal compositions comprising them and to methods of using them to combat and control insect, acarine, mollusc and nematode pests.

Indoline derivatives with pharmaceutical properties are disclosed in for example in GB1237008 and WO2001/074775.

It has now surprisingly been found that certain indolines have insecticidal properties. The present invention therefore provides a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I):

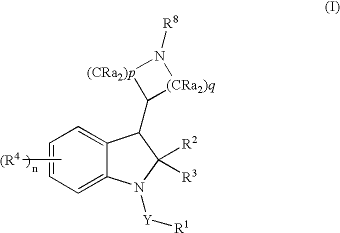

wherein Y is a single bond, C═O, C═S or S(O)ₘ where m is 0, 1 or 2;

R¹ is hydrogen, optionally substituted alkyl, optionally substituted alkoxycarbonyl, optionally substituted alllylcarbonyl, aminocarbonyl, optionally substituted alkylaminocarbonyl, optionally substituted dialkylaminocarbonyl, optionally substituted aryl, optionally substituted heteroaryl, optionally substituted alkoxy, optionally substituted aryloxy, optionally substituted heteroaryloxy, optionally substituted heterocyclyloxy, cyano, optionally substituted alkenyl, optionally substituted alkynyl, optionally substituted cycloalkyl, optionally substituted cycloalkenyl, formyl, optionally substituted heterocyclyl, optionally substituted alkylthio, NO or NR¹³R¹⁴ where R¹³ and R¹⁴ are independently hydrogen, COR¹⁵, optionally substituted alkyl, optionally substituted aryl, optionally substituted heteroazyl, optionally substituted heterocyclyl or R¹³ and R¹⁴ together with the N atom to which they are attached form a group —N═C(R¹⁶)—NR¹⁷R¹⁸; R¹⁵ is H, optionally substituted alkyl, optionally substituted alkoxy, optionally substituted aryl, optionally substituted aryloxy optionally substituted heteroaryl, optionally substituted heteroaryloxy or NR¹⁹R²⁰; R¹⁶, R¹⁷ and R¹⁸ are each independently H or lower alkyl; R¹⁹ and R²⁰ are independently optionally substituted alkyl, optionally substituted aryl or optionally substituted heteroaryl;

R² and R³ are independently hydrogen, halogen, cyano, optionally substituted alkyl, optionally substituted alkoxy or optionally substituted aryl;

each R⁴ is independently halogen, nitro, cyano, optionally substituted C₁₋₈ alkyl, optionally substituted C₂₋₆ alkenyl, optionally substituted C₂₋₆ alkynyl, optionally substituted alkoxycarbonyl, optionally substituted alkylcarbonyl, optionally substituted alkylaminocarbonyl, optionally substituted dialkylaminocarbonyl, optionally substituted C₃₋₇ cycloalkyl, optionally substituted aryl, optionally substituted heteroaryl, optionally substituted heterocyclyl, optionally substituted alkoxy, optionally substituted aryloxy, optionally substituted heteroaryloxy, optionally substituted alkylthio or R²¹R²²N where R²¹ and R²² are, independently, hydrogen, C₁₋₈ alkyl, C₃₋₇ cycloalkyl, C₃₋₆ alkenyl, C₃₋₆ alkynyl, C₃₋₇ cycloalkyl(C₁₋₄)alkyl, C₂₋₆ haloalkyl, C₁₋₆ alkoxy(C₁₋₆)alkyl, C₁₋₆ alkoxycarbonyl or R²¹ and R²² together with the N atom to which they are attached form a five, six or seven-membered heterocyclic ring which may contain one or two further heteroatoms selected from O, N or S and which may be optionally substituted by one or two C₁₋₆ alkyl groups, or 2 adjacent groups R⁴ together with the carbon atoms to which they are attached form a 4, 5, 6, or 7 membered carbocyclic or heterocyclic ring which may be optionally substituted by halogen; n is 0, 1, 2, 3 or 4;

each Ra is independently hydrogen, halogen, hydroxy, cyano, optionally substituted C₁₋₈ alkyl, optionally substituted C₂₋₆ alkenyl, optionally substituted C₂₋₆ alkynyl, optionally substituted alkoxycarbonyl,.optionally substituted alkylcarbonyl, optionally substituted alkylaminocarbonyl, optionally substituted dialkylaminocarbonyl, optionally substituted C₃₋₇ cycloalkyl, optionally substituted aryl, optionally substituted heteroaryl, optionally substituted heterocyclyl, optionally substituted alkoxy, optionally substituted aryloxy, optionally substituted heteroaryloxy, optionally substituted alkylthio, optionally substituted arylthio or R²³R²⁴N where R²³ and R²⁴ are, independently, hydrogen, C₁₋₈ alkyl, C₃₋₇ cycloalkyl, C₃₋₆ alkenyl, C₃₋₆ aynyl, C₃₋₇ cycloalkyl(C₁₋₄)alkyl, C₂₋₆ haloalkyl, C₁₋₆ alkoxy(C₁₋₆)alkyl, C₁₋₆ alkoxycarbonyl or R²³ and R²⁴ together with the N atom to which they are attached form a five, six or seven-membered heterocyclic ring which may contain one or two further heteroatoms selected from O, N or S and which may be optionally substituted by one or two C₁₋₆ alkyl groups, or two Ra groups attached to the same carbon atom are ═O or two Ra groups attached to adjacent carbon atoms form a bond, or two Ra groups together with the carbon atom to which they are attached form a three- to seven-membered ring, that may be saturated or unsaturated, and that may contain one or two hetero atoms selected from the group consisting of N, O and S, and which may be optionally substituted by one or two C₁₋₆ alkyl groups; or two Ra groups together form a group —CH₂—, —CH═CH— or —CH₂CH₂; p is 0, 1, 2, 3, 4, 5 or 6; q is 0, 1, 2, 3, 4, 5 or 6 provided that p+q is 1, 2, 3, 4, 5 or 6;

R⁸ is optionally substituted alkyl, optionally substituted alkenyl, optionally substituted alkynyl, optionally substituted cycloalkyl, optionally substituted aryl, optionally substituted alkoxy, optionally substituted aryloxy, optionally substituted alkoxycarbonyl, optionally substituted alkylcarbonyl or optionally substituted alkenylcarbonyl; or salts or N-oxides thereof.

The compounds of formula (I) may exist in different geometric or optical isomers or tautomeric forms. This invention covers all such isomers and tautomers and mixtures thereof in all proportions as well as isotopic forms such as deuterated compounds.

Each alkyl moiety either alone or as part of a larger group (such as alkoxy, alkoxycarbonyl, alkylcarbonyl, alkylaminocarbonyl, dialkylaminocarbonyl) is a straight or branched chain and is, for example, methyl, ethyl, n-propyl, n-butyl, n-pentyl, n-hexyl, iso-propyl, n-butyl, sec-butyl, iso-butyl, tert-butyl or neo-pentyl. The alkyl groups are suitably C₁ to C₁₂ alkyl groups, but are preferably C₁-C₁₀, more preferably C₁-C₈, even more preferably preferably C₁-C₆ and most preferably C₁-C₄ alkyl groups.

When present, the optional substituents on an alkyl moiety (alone or as part of a larger group such as alkoxy, alkoxycarbonyl, alkylcarbonyl, alkylaminocarbonyl, dialkylaminocarbonyl) include one or more of halogen, nitro, cyano, NCS—, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkylsilyl(C₁₋₆)alkoxy, C₁₋₆ alkoxycarbonyl(C₁₋₁₀)alkoxyi C₁₋₁₀ haloalkoxy, aryl(C₁₋₄)-alkoxy (where the aryl group is optionally substituted), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, SH, C₁₋₁₀ alkylthio, C₁₋₁₀ haloalkylthio, aryl(C₁₋₄)alkylthio (where the aryl group is optionally substituted), C₃₋₇ cycloalkylthio (where the cycloalkyl group is optionally substituted with C₁₋₆ alkyl or halogen), tri(C₁₋₄)alkylsilyl(C₁₋₆)alkylthio, arylthio (where the aryl group is optionally substituted), C₁₋₆ alkylsulfonyl, C₁₋₆ haloalkylsulfonyl, C₁₋₆ alkylsulfinyl, C₁₋₆ haloalkylsulfinyl, arylsulfonyl (where the aryl group may be optionally substituted), tri(C₁₋₄)alkylsilyl, aryldi(C₁₋₄)alkylsilyl, (C₁₋₄)alkyldiarylsilyl, triarylsilyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)aminocarbonyl, N—(C₁₋₃ alkyl)-N—(C₁₋₃ alkoxy)aminocarbonyl, C₁₋₆ alkylcarbonyloxy, arylcarbonyloxy (where the aryl group is optionally substituted), di(C₁₋₆)alkylaminocarbonyloxy, oximes such as ═NOalkyl, ═NOhaloalkyl and ═NOaryl (itself optionally substituted), aryl (itself optionally substituted), heteroaryl (itself optionally substituted), heterocyclyl (itself optionally substituted with C₁₋₆ alkyl or halogen), aryloxy (where the aryl group is optionally substituted), heteroaryloxy, (where the heteroaryl group is optionally substituted), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylaamino, N—(C₁₋₆)alkylcarbonyl-N—(C₁₋₆)alkylamino, C₂₋₆ alkenylcarbonyl, C₂₋₆ alkynylcarbonyl, C₃₋₆ alkenyloxycarbonyl, C₃₋₆ alkynyloxycarbonyl, aryloxycarbonyl (where the aryl group is optionally substituted) and arylcarbonyl (where the aryl group is optionally substituted).

Alkenyl and alkynyl moieties can be in the form of straight or branched chains, and the alkenyl moieties, where appropriate, can be of either the (E)- or (Z)-configuration. Examples are vinyl, allyl and propargyl.

When present, the optional substituents on alkenyl or alkynyl include those optional substituents given above for an alkyl moiety.

In the context of this specification acyl is optionally substituted C₁₋₆ alkylcarbonyl (for example acetyl), optionally substituted C₂₋₆ alkenylcarbonyl, optionally substituted C₂₋₆ allynylcarbonyl, optionally substituted arylcarbonyl (for example benzoyl) or optionally substituted heteroarylcarbonyl.

Halogen is fluorine, chlorine, bromine or iodine.

Haloalkyl groups are alkyl groups which are substituted with one or more of the same or different halogen atoms and are, for example, CF₃, CF₂Cl, CF₃CH₂ or CHF₂CH₂.

In the context of the present specification the terms “aryl” and “aromatic ring system” refer to ring systems which may be mono-, bi- or tricyclic. Examples of such rings include phenyl, naphthalenyl, anthracenyl, indenyl or phenanthrenyl. A preferred aryl group is phenyl. In addition, the terms “heteroaryl”, “heteroaromatic ring” or “heteroaromatic ring system” refer to an aromatic ring system containing at least one heteroatom and consisting either of a single ring or of two or more fused rings. Preferably, single rings will contain up to three and bicyclic systems up to four heteroatoms which will preferably be chosen from nitrogen, oxygen and sulphur. Examples of such groups include furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole quinolinyl, isoquinolinyl, cinnolinyl, phthalazinyl, quinazolinyl, quinoxalinyl, naphthyridinyl, benzotriazinyl, purinyl, pteridinyl and indolizinyl. Preferred examples of heteroaromatic radicals include pyridyl,-pyrimidyl, triazinyl, thienyl, furyl, oxazolyl, isoxazolyl, 2,1,3-benzoxadiazole and thiazolyl.

The terms heterocycle and heterocyclyl refer to a non-arormatic ring containing up to 10 atoms including one or more (preferably one or two) heteroatoms selected from O, S and N. Examples of such rings include 1,3-dioxolane, tetrahydrofuran and morpholine.

When present, the optional substituents on heterocyclyl include C₁₋₆ alkyl and C₁₋₆ haloalkyl as well as those optional substituents given above for an alkyl moiety.

Cycloalkyl includes cyclopropyl, cyclopentyl and cyclohexyl.

Cycloalkenyl includes cyclopentenyl and cyclohexenyl.

When present, the optional substituents on cycloalkyl or cycloalkenyl include C₁₋₃ alkyl as well as those optional substituents given above for an alkyl moiety.

Carbocyclic rings include aryl, cycloalkyl and cycloalkenyl groups.

When present, the optional substituents on aryl or heteroaryl are selected independently, from halogen, nitro, cyano, NCS—, C₁₋₆ alkyl, C₁₋₆ haloalkyl, C₁₋₆ alkoxy-(C₁₋₆)alkyl, C₂₋₆ alkenyl, C₂₋₆ haloalkenyl, C₂₋₆ alkynyl, C₃₋₇ cycloalkyl (itself option substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkyl-silyl(C₁₋₆)alkoxy, C₁₋₆ alkoxycarbonyl(C₁₋₁₀)alkoxy, C₁₋₁₀ haloalkoxy, aryl(C₁₋₄)alkoxy (where the aryl group is optionally substituted with halogen or C₁₋₆ alkyl), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, SH, C₁₋₁₀ alkylthio, C₁₋₁₀ haloalkylthio, aryl(C₁₋₄)alkylthio C₃₋₇ cycloalkylthio (where the cycloalkyl group is optionally substituted with C₁₋₆ alkyl or halogen), tri(C₁₋₄)-alkylsilyl(C₁₋₆)alkylthio, arylthio, C₁₋₆ alkylsulfonyl, C₁₋₆ haloalkylsulfonyl, C₁₋₆ alkylsulfinyl, C₁₋₆ haloalkylsulfinyl, arylsulfonyl, tri(C₁₋₄)alylsilyl, aryldi(C₁₋₄)-alkylsilyl, (C₁₋₄)alkyldiarylsilyl, triarylsilyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)-aminocarbonyl, N—(C₁₋₃ alkyl)-N—(C₁₋₃ alkoxy)aminocarbonyl, C₁₋₆ alkylcarbonyloxy, arylcarbonyloxy, di(C₁₋₆)alkylamino-carbonyloxy, aryl (itself optionally substituted with C₁₋₆ alkyl or halogen), heteroaryl (itself optionally substituted with C₁₋₆ alkyl or halogen), heterocyclyl (itself optionally substituted with C₁₋₆ alkyl or halogen), aryloxy (where the aryl group is optionally substituted with C₁₋₆ alkyl or halogen), heteroaryloxy (where the heteroaryl group is optionally substituted with C₁₋₆ alkyl or halogen), heterocyclyloxy (where the heterocyclyl group is optionally substituted with C₁₋₆ alkyl or halogen), amino, C₁₋₆ alkylamino, di(C₁₋₆)alkylamino; C₁₋₆ alkylcarbonylamnino, N—(C₁₋₆)alkylcarbonyl-N—(C₁₋₆)alkylamino, arylcarbonyl, (where the aryl group is itself optionally substituted with halogen or C₁₋₆ alkyl) or two adjacent positions on an aryl or heteroaryl system may be cyclised to form a 5, 6 or 7 membered carbocyclic or heterocyclic ring, itself optionally substituted with halogen or C₁₋₆ alkyl. Further substituents for aryl or heteroaryl include aryl carbonyl amino (where the aryl group is substituted by C₁₋₆ alkyl or halogen), (C₁₋₆)alkyloxycarbonylamino (C₁₋₆)alkyloxycarbonyl-N—(C₁₋₆)alkylamino, aryloxycarbonylamino (where the aryl group is substituted by C₁₋₆ alkyl or halogen), aryloxycarbonyl-N—(C₁₋₆)alkylamino, (where the aryl group is substituted by C₁₋₆ alkyl or halogen), arylsulphonylamino (where the aryl group is substituted by C₁₋₆ alkyl or halogen), arylsulphonyl-N—(C₁₋₆)alkylamino (where the aryl group is substituted by C₁₋₆ alkyl or halogen), aryl-N—(C₁₋₆)alkylamino (where the aryl group is substituted by C₁₋₆ alkyl or halogen), arylamino (where the aryl group is substituted by C₁₋₆ alkyl or halogen), heteroaryl amino (where the heteroaryl group is substituted by C₁₋₄ aylyl or halogen), heterocyclylamino (where the heterocyclyl group is substituted by C₁₋₆ alkyl or halogen), aminocarbonylamino, C₁₋₆ alkylaminocarbonyl amino, di(C₁₋₆)alkylaminocarbonyl amino, arylaminocarbonyl amino where the aryl group is substituted by C₁₋₆ alkyl or halogen), aryl-N—(C₁₋₆)alkylarminocarbonylamino where the aryl group is substituted by C₁₋₆ alkyl or halogen), C₁₋₆ alkylaminocarbonyl-N—(C₁₋₆)alkyl amino, di(C₁₋₆)alkylaminocarbonyl-N—(C₁₋₆)alkyl amino, arylaminocarbonyl-N—(C₁₋₆)alkyl amino where the aryl group is substituted by C₁₋₆ alkyl or halogen) and aryl-N—(C₁₋₆)alkylaminocarbonyl-N—(C₁₋₆)alkyl amino where the aryl group is substituted by C₁₋₆ alkyl or halogen).

For substituted phenyl moieties, heterocyclyl and heteroaryl groups it is preferred that one or more substituents are independently selected from halogen, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkylthio, C₁₋₆ haloalkylthio, C₁₋₆ alkylsulfinyl, C₁₋₆ haloalkylsulfinyl, C₁₋₆ alkylsulfonyl, C₁₋₆ haloalkylsulfonyl, C₂₋₆ alkenyl, C₂₋₆ haloalkenyl, C₂₋₆ alynyl, C₃₋₇ cycloalkyl, nitro, cyano, CO₂H, C₁₋₆ alkylcarbonyl, alkoxycarbonyl, R²⁵R²⁶N or R²⁷R²⁸NC(O); wherein R²⁵, R²⁶, R²⁷ and R²⁸ are, independe hydrogen or C₁₋₆ alkyl. Further preferred substituents are aryl and heteroaryl groups.

Haloalkenyl groups are alkenyl groups which are substituted with one or more of the same or different halogen atoms.

It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected (C₁₋₆)alkyl groups. When heterocyclic rings are formed by joining two groups on an N atom, the resulting rings are suitably pyrrolidine, piperidine, thiomorpholine and morpholine each of which maybe substituted by one or two independently selected (C₁₋₆) alkyl groups.

Preferably the optional substituents on an alkyl moiety include one or more of halogen, nitro, cyano, HO₂C, C₁₋₁₀ alkoxy (itself optionally substituted by C₁₋₁₀ alkoxy), aryl(C₁₋₄)alkoxy, C₁₋₁₀ alkylthio, C₁₋₁₀ alkylcarbonyl, C₁₋₁₀ alkoxycarbonyl, C₁₋₆ alkylaminocarbonyl, di(C₁₋₆ alkyl)aminocarbonyl, (C₁₋₆)alkylcarbonyloxy, optionally substituted phenyl, heteroaryl, aryloxy, arylcarbonyloxy, heteroaryloxy, heterocyclyl, heterocyclyloxy, C₃₋₇ cycloalkyl (itself optionally substituted with (C₁₋₆)alkyl or halogen), C₃₋₇ cycloalkyloxy, C₅₋₇ cycloalkenyl, C₁₋₆ alkylsulfonyl, C₁₋₆ alkylsulfinyl, tri(C₁₋₄)alkylsilyl, tri(C₁₋₄)alkylsilyl(C₁₋₆)alkoxy, aryldi(C₁₋₄)alkylsilyl, (C₁₋₄)alkyldiarylsilyl and triarylsilyl.

Preferably the optional substituents on alkenyl or alkynyl include one or more of halogen, aryl and C₃₋₇ cycloalkyl.

A preferred optional substituent for heterocyclyl is C₁₋₆ alkyl.

Preferably the optional substituents for cycloalkyl include halogen, cyano and C₁₋₃ alkyl.

Preferably the optional substituents for cycloalkenyl include C₁₋₃ alkyl, halogen and cyano.

Preferably Y is a single bond, C═O or S(O)m where m is 0, 1 or 2.

More preferably Y is a single bond, C═O or SO₂.

Yet more preferably Y is a single bond or C═O.

Most preferably Y is C═O.

Preferably R¹ is hydrogen, C₁₋₆ alkyl, C₁₋₆ cyanoalkyl, C₁₋₆ haloalkyl, C₃₋₇ cycloalkyl(C₁₋₄)alkyl, C₁₋₆ alkoxy(C₁₋₆)alkyl, heteroaryl(C₁₋₆)alkyl (wherein the heteroaryl group may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl, or two adjacent positions on the heteroaryl system may be cyclised to form a 5, 6 or 7 membered carbocyclic or heterocyclic ring, itself optionally substituted with halogen), aryl(C₁₋₆)alkyl (wherein the aryl group may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl, or two adjacent positions on the aryl system may be cyclised to form a 5, 6 or 7 membered carbocyclic or heterocyclic ring, itself optionally substituted with halogen), C₁₋₆ alkylcarbonylamino(C₁₋₆)alkyl, aryl (which may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl, or two adjacent positions on the aryl system may be cyclised to form a 5, 6 or 7 membered carbocyclic or heterocyclic ring, itself optionally substituted with halogen), heteroaryl (which may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl, or two adjacent positions on the heteroaryl system may be cyclised to form a 5, 6 or 7 membered carbocyclic or heterocyclic ring, itself optionally substituted with halogen), C₁₋₄ alkoxy, C₁₋₆ haloalkoxy, phenoxy (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryloxy (optionally substituted by halo, nitro, cyano, C₁₋₆ alkyl, C₁₋₆ haloalkyl, C₁₋₆ alkoxy or C₁₋₆ haloalkoxy), heterocyclyloxy (optionally substituted by halo, C₁₋₆ alkyl, C₁₋₆ haloalkyl, C₁₋₆ alkoxy or C₁₋₆ haloalkoxy), cyano, C₂₋₆ alkenyl, C₂₋₆ alkynyl, C₃₋₆ cycloalkyl, C₅₋₇ cycloalkenyl, heterocyclyl (optionally substituted by halo, nitro, cyano, C₁₋₆ alkyl, C₁₋₆ haloalkyl, C₁₋₆ alkoxy or C₁₋₆ haloalkoxy), C₁₋₆ alkylthio, C₁₋₆ haloalkylthio or NR¹³R¹⁴ where R¹³ and R¹⁴ are independently hydrogen, C₁₋₆ alkyl, C₁₋₆ haloalkyl, C₁₋₆ alkoxy(C₁₋₆)alkyl, phenyl (which may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino or C₁₋₄ alkoxycarbonyl), phenyl (C₁₋₆)alkyl (wherein the phenyl group maybe optionally substituted by halogen, C₁₋₄alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl, or two adjacent positions on the phenyl ring may be cyclised to form a 5, 6 or 7 membered carbocyclic or heterocyclic ring, itself optionally substituted with halogen), heteroaryl (C₁₋₆)alkyl (wherein the heteroaryl group may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl, or two adjacent positions on the heteroaryl system may be cyclised to form a 5, 6 or 7 membered carbocyclic or heterocyclic ring, itself optionally substituted with halogen) or heteroaryl (which may be optionally substituted by halo, nitro, cyano, C₁₋₆ alkyl, C₁₋₆ haloalkyl, C₁₋₆ alkoxy or C₁₋₆ haloalkoxy, C₁₋₄ alkoxycarbonyl C₁₋₆ alkylcarbonylamino, phenyloxycarbonylamino (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), amino, C₁₋₆ alkylamino or phenylamino (wherein the phenyl group is optionally substituted halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino)).

More preferably R¹ is C₁₋₆ alkyl, C₁₋₆ haloalkyl, C₁₋₆ alkoxy(C₁₋₆)alkyl, heteroaryl(C₁₋₃)alkyl (wherein the heteroaryl group may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkoxycarbonyl, or two adjacent positions on the heteroaryl system may be cyclised to form a 5, 6 or 7 membered carbocyclic or heterocyclic ring, itself optionally substituted with halogen), phenyl(C₁₋₃)alkyl (wherein the phenyl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl, or two adjacent positions on the phenyl ring may be cyclised to form a 5, 6 or 7 membered carbocyclic or heterocyclic ring, itself optionally substituted with halogen), phenyl (which may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl, or two adjacent positions on the phenyl ring may be cyclised to form a 5, 6 or 7 membered carbocyclic or heterocyclic ring, itself optionally substituted with halogen), heteroaryl (which may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkoxycarbonyl, or two adjacent positions on the heteroaryl system may be cyclised to form a 5, 6 or 7 membered carbocyclic or heterocyclic ring, itself optionally substituted with halogen), C₁₋₆ alkoxy, C₁₋₆ haloalkoxy, C₂₋₆ alkenyl, heterocyclyl (optionally substituted by halo, cyano, C₁₋₆ alkyl, C₁₋₆ haloalkyl, C₁₋₆ alkoxy or C₁₋₆ haloalkoxy), C₁₋₆ alkylthio, C₁₋₆ haloalkylthio or NR¹³R¹⁴ where R¹³ and R¹⁴ are independently hydrogen, C₁₋₆ alkyl or C₁₋₆ haloalkyl, C₁₋₆ alkoxy(C₁₋₆)alkyl, C₂₋₆ alkylcarbonyl, phenylcarbonyl, (where the phenyl is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), phenyl(C₁₋₃)alkyl (wherein the phenyl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆-alkoxycarbonyl, or two adjacent positions on the phenyl ring may be cyclised to form a 5, 6 or 7 membered carbocyclic or heterocyclic ring, itself optionally substituted with halogen) or heteroaryl(C₁₋₃)alkyl (wherein the heteroaryl group may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl, or two adjacent positions on the heteroaryl system may be cyclised to form a 5, 6 or 7 membered carbocyclic or heterocyclic ring, itself optionally substituted with halogen).

Even more preferably R¹ is C₁₋₆ alkyl, C₁₋₆ haloalkyl, heteroaryl(C₁₋₃)alkyl (wherein the heteroaryl group may be optionally substituted by halo, cyano, C₁₋₆ alkyl, C₁₋₆ haloalkyl and where the heteroaryl group is a thiazole, pyridine, pyrimidine, pyrazine or pyridazine ring), heteroaryl (optionally substituted by halo, cyano, C₁₋₆ alkyl, C₁₋₆ haloalkyl and where the heteroaryl group is a pyridine, pyrimidine, 2,1,3-benzoxadiazole, pyrazine or pyridazine ring), C₁₋₆ alkoxy, C₁₋₆ alkoxy(C₁₋₆)alkyl, C₁₋₆ alkylamino, C₁₋₆ alkoxy(C₁₋₆)alkylamino or heteroaryl(C₁₋₃)alkylamino (wherein the heteroaryl group may be optionally substituted by halo, cyano, C₁₋₆ alkyl, C₁₋₆ haloalkyl and where the heteroaryl group is a thiazole, pyridine, pyrimidine, pyrazine or pyridazine ring).

Most preferably R¹ is pyridyl (optionally substituted by halo, C₁₋₃ alkyl or C₁₋₃ haloalkyl) especially halo-substituted pyridyl.

It is preferred that R² and R³ are independently hydrogen, C₁₋₆ alkyl, C₁₋₆ haloalkyl, C₁₋₆ alkoxy or cyano.

More preferably R² and R³ are independently hydrogen, halogen, C₁₋₂ alkyl, C₁₋₂ haloalkyl, C₁₋₂ alkoxy, cyano.

Even more preferably R² and R³ are independently hydrogen or C₁₋₄ alkyl.

Yet more preferably R² and R³ are independently hydrogen or methyl.

Most preferably R² and R³ are both hydrogen.

Preferably each R⁴ is independently halogen, cyano, C₁₋₈ alkyl, C₁₋₈ haloalkyl, C₁₋₆ cyanoalkyl, C₁₋₆ alkoxy(C₁₋₆)alkyl, C₃₋₇ cycloalkyl(C₁₋₆)alkyl, C₅₋₆ cycloalkenyl(C₁₋₆)alkyl, C₃₋₆ alkenyloxy(C₁₋₆)alkyl, C₃₋₆ alkynyloxy(C₁₋₆)alkyl, aryloxy(C₁₋₆)alkyl, C₁₋₆ carboxyalkyl, C₁₋₆ alkylcarbonyl(C₁₋₆)alkyl, C₂₋₆ alkenylcarbonyl(C₁₋₆)alkyl, C₂₋₆ alkynylcarbonyl(C₁₋₆)-alkyl, C₁₋₆ alkoxycarbonyl(C₁₋₆)alkyl, C₃₋₆ alkenyloxycarbonyl(C₁₋₆)alkyl, C₃₋₆ alkynyloxycarbonyl(C₁₋₆)alkyl, aryloxycarbonyl(C₁₋₆)alkyl, C₁₋₆ alkylthio(C₁₋₆)alkyl, C₁₋₆ alkylsulfinyl(C₁₋₆)alkyl, C₁₋₆ alkylsulfonyl(C₁₋₆)alkyl, aminocarbonyl(C₁₋₆)alkyl, C₁₋₆ alkylaminocarbonyl(C₁₋₆)alkyl, di(C₁₋₆)alkylaminocarbonyl(C₁₋₆)alkyl, phenyl(C₁₋₄)alkyl (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryl(C₁₋₄)alkyl (wherein the heteroaryl group is optionally substituted by halo, nitro, cyano, C₁₋₆ alkyl, C₁₋₆ haloalkyl, C₁₋₆ alkoxy or C₁₋₆ haloalkoxy), heterocyclyl(C₁₋₄)alkyl (wherein the heterocyclyl group is optionally substituted by halo, nitro, cyano, C₁₋₆ alkyl, C₁₋₆ haloalkyl, C₁₋₆ alkoxy or C₁₋₆ haloalkoxy), C₂₋₆ alkenyl, aminocarbonyl(C₂₋₆)alkenyl, C₁₋₆ alkylaminocarbonyl(C₂₋₆)alkenyl, di(C₁₋₆)alkylaminocarbonyl(C₂₋₆)alkenyl, phenyl(C₂₋₄)-alkenyl, (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), C₂₋₆ alkynyl, trriethylsiyl(C₂₋₆alkynyl, aminocarbonyl(C₂₋₆)alkynyl, C₁₋₆ alkylaminocarbonyl(C₂₋₆)alkynyl, di(C₁₋₆)alkylarinocarbonyl(C₂₋₆)alkynyl, C₁₋₆ alkoxycarbonyl, C₃₋₇ cycloalkyl, C₃₋₇ halocycloalkyl, C₃₋₇ cyanocycloalkyl, C₁₋₃ alkyl(C₃₋₇)-cycloalkyl, C₁₋₃ alkyl(C₃₋₇)halocycloalkyl,phenyl (optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryl (optionally substituted by halo, nitro, cyano, C₁₋₆ alkyl, C₁₋₆ haloalkyl, C₁₋₆ alkoxy or C₁₋₆ haloalkoxy), heterocyclyl (wherein the heterocyclyl group is optionally substituted by halo, nitro, cyano, C₁₋₆ alkyl, C₁₋₆ haloalkyl, C₁₋₆ alkoxy or Cl-6 haloalkoxy), or 2 adjacent groups R⁴ together with the carbon atoms to which they are attached form a 4, 5, 6 or 7 membered carbocylic or heterocyclic ring which may be optionally substituted by halogen, C₁₋₈ alkoxy, C₁₋₆ haloalkoxy, phenoxy (optionally substituted by halo, nitro, cyano, C₁₋₆ alkyl, C₁₋₆ haloalkyl, C₁₋₆ alkoxy or C₁₋₆ haloalkoxy), heteroaryloxy (optionally substituted by halo, nitro, cyano, C₁₋₆ alkyl, C₁₋₆ haloalkyl, C₁₋₆ alkoxy or C₁₋₆ haloalkoxy), C₁₋₈ alkylthio or R¹⁹R²⁰N where R¹⁹ and R²⁰ are, independently, hydrogen, C₁₋₈ alkyl, C₃₋₇ cycloalkyl, C₃₋₆ alkenyl, C₃₋₆ alkynyl, C₂₋₆ haloalkyl, C₁₋₆ alkoxycarbonyl or R¹⁹ and R²⁰ together with the N atom to which they are attached form a five, six or seven-membered heterocyclic ring which may contain one or two further heteroatoms selected from O, N or S and which may be optionally substituted by one or two C₁₋₆ alkyl groups; n is 0, 1, 2 or 3.

More preferably each R⁴ is independently halogen, cyano, C₁₋₈ alkyl, C₁₋₈ haloalkyl, C₁₋₈ cyanoalkyl, C₁₋₆ alkoxy(C₁₋₆)alkyl, C₂₋₆ alkynyl, trimethylsilyl(C₂₋₆)alkynyl, C₁₋₆ alkoxycarbonyl, C₃₋₇ cycloalkyl, C₁₋₃ alkyl (C₃₋₇) cycloalkyl, phenyl (optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heterocyclyl (optionally substituted by halo, nitro, cyano, C₁₋₆ alkyl, C₁₋₆ haloalkyl, C₁₋₆ alkoxy or C₁₋₆ haloalkoxy), C₁₋₈ alkoxy, C₁₋₆ haloalkoxy, phenoxy (optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryloxy (optionally substituted by halo, nitro, cyano, C₁₋₃ alkyl, C₁₋₃ haloalkyl, C₁₋₃ alkoxy or C₁₋₃ haloalkoxy), di(C₁₋₈)alkylamino, or 2 adjacent groups R⁴ together with the carbon- atoms to which they are attached form a 4, 5, 6 or 7 membered carbocylic or heterocyclic ring which may be optionally substituted by halogen; n is 0, 1, 2 or 3.

Even more preferably each R⁴ is independently halogen, cyano, C₁₋₈ alkyl, C₁₋₈ haloalkyl, C₁₋₈ cyanoalkyl, C₁₋₆ alkoxy(C₁₋₆)alkyl, C₂₋₆ alkynyl, heterocyclyl (optionally substituted by C₁₋₆ alkyl), C₁₋₈ alkoxy, C₁₋₆ haloalkoxy, phenoxy (optionally substituted by halo, cyano, C₁₋₃ alkyl or C₁₋₃ haloalkyl), heteroaryloxy (optionally substituted by halo, cyano, C₁₋₃ alkyl or C₁₋₃ haloalkyl), di(C₁₋₈)alkylamino or 2 adjacent groups R⁴ together with the carbon atoms to which they are attached form a 4, 5, 6 or 7 membered carbocylic or heterocyclic ring which may be optionally substituted by halogen; n is 0, 1, 2 or 3.

Yet more preferably each R⁴ is independently fluoro, chloro, bromo, cyano, C₁₋₄ alkyl, C₁₋₄ haloalkyl, C₁₋₄ cyanoalkyl or C₁₋₃ alkoxy(C₁₋₃)alkyl; n is 0, 1 or 2.

Most preferably each R⁴ is independently fluoro, chloro, bromo, C₁₋₄ alkyl or C₁₋₄ haloalkyl; n is 1 or 2.

Preferably R⁸ is C₁₋₁₀ alkyl, C₁₋₁₀ haloalkyl, aryl(C₁₋₆)alkyl (wherein the aryl group is optionally substituted by halogen, C₁₋₄ alkyl, C₁₋₄ alkoxy, C₁₄ haloalkyl, C₁₋₄ haloalkoxy, CN, NO₂, aryl, heteroaryl, amino or dialkylamino), heteroaryl(C₁₋₆)alkyl (wherein the heteroaryl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), arylcarbonyl-(C₁₋₆)alkyl (wherein the aryl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino and the alkyl group may be optionally substituted by aryl), C₂₋₈ alkenyl, C₂₋₈ haloalkenyl, aryl(C₂₋₆)-alkenyl (wherein the aryl group is optionally substituted halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino, C₁₋₆ alkoxycarbonyl, or two adjacent substituents can cyclise to form a 5, 6 or 7 membered carbocyclic or heterocyclic ring), heteroaryl(C₂₋₆)-alkenyl (wherein the heteroaryl group is optionally substituted halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino, C₁₋₆ alkoxycarbonyl, or two adjacent substituents can cyclise to form a 5, 6 or 7 membered carbocyclic or heterocyclic ring), C₂₋₆ alkynyl, phenyl(C₂₋₆)alkynyl (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), C₃₋₇ cycloalkyl, C₁₋₆ alkoxycarbonyl, C₁₋₆ alkylcarbonyl, C₁₋₆ haloalkylcarbonyl or aryl(C₂₋₆)alkenylcarbonyl (wherein the aryl group may be optionally substituted halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), or —C(R⁵¹)(R⁵²)—[CR⁵³═CR⁵⁴]z-R⁵⁵ where z is 1 or 2, R⁵¹ and R⁵² are each independently H, halo or C₁₋₂ alkyl, R⁵³ and R⁵⁴ are each independently H, halogen, C₁₋₄ alkyl or C₁₋₄ haloalkyl and R⁵⁵ is optionally substituted aryl or optionally substituted heteroaryl.

More preferably R⁸ is phenyl(C₁₋₄)alkyl (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryl(C₁₋₆)alkyl (wherein the heteroaryl group is optionally substituted halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), phenyl(C₂₋₆)alkenyl (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryl(C₂₋₆)alkenyl (wherein the heteroaryl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino) or phenyl(C₂₋₆)alkynyl (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino, or —C(R⁵¹)(R⁵²)—[CR⁵³═CR⁵⁴]z-R⁵⁵ where z is 1 or 2, R⁵¹ and R⁵² are each independently H, halo or C₁₋₂ alkyl, R⁵³ and R⁵⁴ are each independently H, halogen, C₁₋₄ alkyl or C₁₋₄ haloalkyl and R⁵⁵ is optionally substituted aryl or optionally substituted heteroaryl.

Most preferably R⁸ is —C(R⁵¹)(R⁵²)—[CR⁵³═CR⁵⁴]z-R⁵⁵ where z is 1 or 2, preferably 1, R⁵¹ and R⁵² are each independently H, halo or C₁₋₂ alkyl, R⁵³ and R⁵⁴ are each independently H, halogen, C₁₋₄ alkyl or C₁₋₄haloalkyl and R⁵⁵ is phenyl substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino or heteroaryl substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino.

R⁵¹ and R⁵² are preferably hydrogen.

R⁵³ and R⁵⁴ are preferably hydrogen or halogen, especially hydrogen. R⁵⁵ is preferably phenyl substituted with one to three substituents selected from halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino.

Preferably each Ra is independently hydrogen, halo, cyano, C₁₋₃ alkyl, hydroxy or two Ra groups together with the carbon atom to which they are attached form a carbonyl group

More preferably each Ra is independently hydrogen, fluoro, methyl, hydroxy or two Ra groups together with the carbon atom to which they are attached form a carbonyl group

Most preferably each Ra is hydrogen.

Preferably p is 1, 2 or 3 and q is 1, 2 or 3 and p+q is 3, 4 or 5.

More preferably p is 1 or 2 and q is 2.

Most preferably p and q are both 2.

One group of preferred compounds of formula (I) are those where Y is C(O) and R¹ is NR¹³R¹⁴ where R¹³ and R¹⁴ are as defined above.

Certain compounds of formula (I) are novel and as such form a further aspect of the invention. One group of novel compounds are those compounds of formula I′ 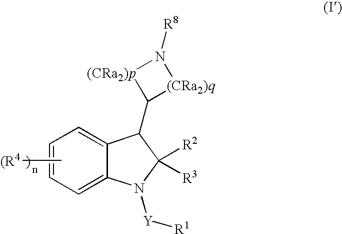
wherein Y is CO, R² and R³ are both hydrogen and R¹, R⁴, R⁸, R^{a}, n, p and q are as defined in relation to formula I provided that when n is 0 and R¹ is CH₃ then the ring containing (CRa2)p and C(Ra2)q is not 1,2,3,6-tetrahydro-1,4-dimethyl-2-pyridinyl, 1,2,5,6-tetrahydro-1,4-dimethyl-2-pyridinyl or 2-pyrrolidinyl.

The compounds in Tables I to CCLXVIII below illustrate the compounds of the invention.

Table I provides 782 compounds of formula Ia

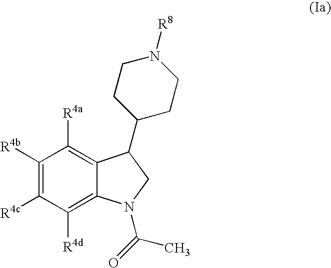

wherein the values of R⁸, R^{4a}, R^{4b}, R^{4c} and R^{4d} are given in Table 1

TABLE 1

| Compound No | R⁸ | R^{4a} | R^{4b} | R^{4c} | R^{4d} |
|---|---|---|---|---|---|
| I-1 | 4-chlorobenzyl | H | H | H | H |
| I-2 | Cinnamyl | H | H | H | H |
| I-3 | 4-chlorocinnamyl | H | H | H | H |
| I-4 | 4-fluorocinnamyl | H | H | H | H |
| I-5 | 4-bromocinnamyl | H | H | H | H |
| I-6 | 4-trifluoromethylcinnamyl | H | H | H | H |
| I-7 | 4-trifluoromethoxycinnamyl | H | H | H | H |
| I-8 | 4-pentafluoroethoxycinnamyl | H | H | H | H |
| I-9 | 4-methoxycinnamyl | H | H | H | H |
| I-10 | 4-ethoxycinnamyl | H | H | H | H |
| I-11 | 4-cyanocinnamyl | H | H | H | H |
| I-12 | 3-(6-chloro-pyridin-3-yl)-allyl | H | H | H | H |
| I-13 | 3-(4-chlorophenyl)-but-2-enyl | H | H | H | H |
| I-14 | 3-(4-chlorophenyl)-3-fluoro-allyl | H | H | H | H |
| I-15 | 3-chloro-4-fluoro-cinnamyl | H | H | H | H |
| I-16 | 3,5-dichloro-cinnamyl | H | H | H | H |
| I-17 | 5-phenyl-penta-2,4-dienyl | H | H | H | H |
| I-18 | 4-isopropyloxycarbonylamino-cinnamyl | H | H | H | H |
| I-19 | 3-naphthalen-2-yl-allyl | H | H | H | H |
| I-20 | 3-(5-trifluoromethyl-pyridin-2-yl)-allyl | H | H | H | H |
| I-21 | 3-(5-chloro-pyridin-2-yl)-allyl | H | H | H | H |
| I-22 | 3-pyridin-4-yl)-allyl | H | H | H | H |
| I-23 | 3-(2-Chloro-pyridin-4-yl)-allyl | H | H | H | H |
| I-24 | 4-chlorobenzyl | H | F | H | H |
| I-25 | Cinnamyl | H | F | H | H |
| I-26 | 4-chlorocinnamyl | H | F | H | H |
| I-27 | 4-fluorocinnamyl | H | F | H | H |
| I-28 | 4-bromocinnamyl | H | F | H | H |
| I-29 | 4-trifluoromethylcinnamyl | H | F | H | H |
| I-30 | 4-trifluoromethoxycinnamyl | H | F | H | H |
| I-31 | 4-pentafluoroethoxycinnamyl | H | F | H | H |
| I-32 | 4-methoxycinnamyl | H | F | H | H |
| I-33 | 4-ethoxycinnamyl | H | F | H | H |
| I-34 | 4-cyanocinnamyl | H | F | H | H |
| I-35 | 3-(6-chloro-pyridin-3-yl)-allyl | H | F | H | H |
| I-36 | 3-(4-chlorophenyl)-but-2-enyl | H | F | H | H |
| I-37 | 3-(4-chlorophenyl)-3-fluoro-allyl | H | F | H | H |
| I-38 | 3-chloro-4-fluoro-cinnamyl | H | F | H | H |
| I-39 | 3,5-dichloro-cinnamyl | H | F | H | H |
| I-40 | 5-phenyl-penta-2,4-dienyl | H | F | H | H |
| I-41 | 4-isopropyloxycarbonylamino-cinnamyl | H | F | H | H |
| I-42 | 3-naphthalen-2-yl-allyl | H | F | H | H |
| I-43 | 3-(5-trifluoromethyl-pyridin-2-yl)-allyl | H | F | H | H |
| I-44 | 3-(5-chloro-pyridin-2-yl)-allyl | H | F | H | H |
| I-45 | 3-pyridin-4-yl-allyl | H | F | H | H |
| I-46 | 3-(2-Chloro-pyridin-4-yl)-allyl | H | F | H | H |
| I-47 | 4-chlorobenzyl | H | Cl | H | H |
| I-48 | Cinnamyl | H | Cl | H | H |
| I-49 | 4-chlorocinnamyl | H | Cl | H | H |
| I-50 | 4-fluorocinnamyl | H | Cl | H | H |
| I-51 | 4-bromocinnamyl | H | Cl | H | H |
| I-52 | 4-trifluoromethylcinnamyl | H | Cl | H | H |
| I-53 | 4-trifluoromethoxycinnamyl | H | Cl | H | H |
| I-54 | 4-pentafluoroethoxycinnamyl | H | Cl | H | H |
| I-55 | 4-methoxycinnamyl | H | Cl | H | H |
| I-56 | 4-ethoxycinnamyl | H | Cl | H | H |
| I-57 | 4-cyanocinnamyl | H | Cl | H | H |
| I-58 | 3-(6-chloro-pyridin-3-yl)-allyl | H | Cl | H | H |
| I-59 | 3-(4-chlorophenyl)-but-2-enyl | H | Cl | H | H |
| I-60 | 3-(4-chlorophenyl)-3-fluoro-allyl | H | Cl | H | H |
| I-61 | 3-chloro-4-fluoro-cinnamyl | H | Cl | H | H |
| I-62 | 3,5-dichloro-cinnamyl | H | Cl | H | H |
| I-63 | 5-phenyl-penta-2,4-dienyl | H | Cl | H | H |
| I-64 | 4-isopropyloxycarbonylamino-cinnamyl | H | Cl | H | H |
| I-65 | 3-naphthalen-2-yl-allyl | H | Cl | H | H |
| I-66 | 3-(5-trifluoromethyl-pyridin-2-yl)-allyl | H | Cl | H | H |
| I-67 | 3-(5-chloro-pyridin-2-yl)-allyl | H | Cl | H | H |
| I-68 | 3-pyridin-4-yl-allyl | H | Cl | H | H |
| I-69 | 3-(2-Chloro-pyridin-4-yl)-allyl | H | Cl | H | H |
| I-70 | 4-chlorobenzyl | H | Br | H | H |
| I-71 | Cinnamyl | H | Br | H | H |
| I-72 | 4-chlorocinnamyl | H | Br | H | H |
| I-73 | 4-fluorocinnamyl | H | Br | H | H |
| I-74 | 4-bromocinnamyl | H | Br | H | H |
| I-75 | 4-trifluoromethylcinnamyl | H | Br | H | H |
| I-76 | 4-trifluoromethoxycinnamyl | H | Br | H | H |
| I-77 | 4-pentafluoroethoxycinnamyl | H | Br | H | H |
| I-78 | 4-methoxycinnamyl | H | Br | H | H |
| I-79 | 4-ethoxycinnamyl | H | Br | H | H |
| I-80 | 4-cyanocinnamyl | H | Br | H | H |
| I-81 | 3-(6-chloro-pyridin-3-yl)-allyl | H | Br | H | H |
| I-82 | 3-(4-chlorophenyl)-but-2-enyl | H | Br | H | H |
| I-83 | 3-(4-chlorophenyl)-3-fluoro-allyl | H | Br | H | H |
| I-84 | 3-chloro-4-fluoro-cinnamyl | H | Br | H | H |
| I-85 | 3,5-dichloro-cinnamyl | H | Br | H | H |
| I-86 | 5-phenyl-penta-2,4-dienyl | H | Br | H | H |
| I-87 | 4-isopropyloxycarbonylamino-cinnamyl | H | Br | H | H |
| I-88 | 3-naphthalen-2-yl-allyl | H | Br | H | H |
| I-89 | 3-(5-trifluoromethyl-pyridin-2-yl)-allyl | H | Br | H | H |
| I-90 | 3-(5-chloro-pyridin-2-yl)-allyl | H | BR | H | H |
| I-91 | 3-pyridin-4-yl-allyl | H | Br | H | H |
| I-92 | 3-(2-Chloro-pyridin-4-yl)-allyl | H | Br | H | H |
| I-93 | 4-chlorobenzyl | H | CN | H | H |
| I-94 | Cinnamyl | H | CN | H | H |
| I-95 | 4-chlorocinnamyl | H | CN | H | H |
| I-96 | 4-fluorocinnamyl | H | CN | H | H |
| I-97 | 4-bromocinnamyl | H | CN | H | H |
| I-98 | 4-trifluoromethylcinnamyl | H | CN | H | H |
| I-99 | 4-trifluoromethoxycinnamyl | H | CN | H | H |
| I-100 | 4-pentafluoroethoxycinnamyl | H | CN | H | H |
| I-101 | 4-methoxycinnamyl | H | CN | H | H |
| I-102 | 4-ethoxycinnamyl | H | CN | H | H |
| I-103 | 4-cyanocinnamyl | H | CN | H | H |
| I-104 | 3-(6-chloro-pyridin-3-yl)-allyl | H | CN | H | H |
| I-105 | 3-(4-chlorophenyl)-but-2-enyl | H | CN | H | H |
| I-106 | 3-(4-chlorophenyl)-3-fluoro-allyl | H | CN | H | H |
| I-107 | 3-chloro-4-fluoro-cinnamyl | H | CN | H | H |
| I-108 | 3,5-dichloro-cinnamyl | H | CN | H | H |
| I-109 | 5-phenyl-penta-2,4-dienyl | H | CN | H | H |
| I-110 | 4-isopropyloxycarbonylamino-cinnamyl | H | CN | H | H |
| I-111 | 3-naphthalen-2-yl-allyl | H | CN | H | H |
| I-112 | 3-(5-trifluoromethyl-pyridin-2-yl)-allyl | H | CN | H | H |
| I-113 | 3-(5-chloro-pyridin-2-yl)-allyl | H | CN | H | H |
| I-114 | 3-pyridin-4-yl-allyl | H | CN | H | H |
| I-115 | 3-(2-Chloro-pyridin-4-yl)-allyl | H | CN | H | H |
| I-116 | 4-chlorobenzyl | H | OMe | H | H |
| I-117 | Cinnamyl | H | OMe | H | H |
| I-118 | 4-chlorocinnamyl | H | OMe | H | H |
| I-119 | 4-fluorocinnamyl | H | OMe | H | H |
| I-120 | 4-bromocinnamyl | H | OMe | H | H |
| I-121 | 4-trifluoromethylcinnamyl | H | OMe | H | H |
| I-122 | 4-trifluoromethoxycinnamyl | H | OMe | H | H |
| I-123 | 4-pentafluoroethoxycinnamyl | H | OMe | H | H |
| I-124 | 4-methoxycinnamyl | H | OMe | H | H |
| I-125 | 4-ethoxycinnamyl | H | OMe | H | H |
| I-126 | 4-cyanocinnamyl | H | OMe | H | H |
| I-127 | 3-(6-chloro-pyridin-3-yl)-allyl | H | OMe | H | H |
| I-128 | 3-(4-chlorophenyl)-but-2-enyl | H | OMe | H | H |
| I-129 | 3-(4-chlorophenyl)-3-fluoro-allyl | H | OMe | H | H |
| I-130 | 3-chloro-4-fluoro-cinnamyl | H | OMe | H | H |
| I-131 | 3,5-dichloro-cinnamyl | H | OMe | H | H |
| I-132 | 5-phenyl-penta-2,4-dienyl | H | OMe | H | H |
| I-133 | 4-isopropyloxycarbonylamino-cinnamyl | H | OMe | H | H |
| I-134 | 3-naphthalen-2-yl-allyl | H | OMe | H | H |
| I-135 | 3-(5-trifluoromethyl-pyridin-2-yl)-allyl | H | OMe | H | H |
| I-136 | 3-(5-chloro-pyridin-2-yl)-allyl | H | OMe | H | H |
| I-137 | 3-pyridin-4-yl-allyl | H | OMe | H | H |
| I-138 | 3-(2-Chloro-pyridin-4-yl)-allyl | H | OMe | H | H |
| I-139 | 4-chlorobenzyl | H | OCF₃ | H | H |
| I-140 | Cinnamyl | H | OCF₃ | H | H |
| I-141 | 4-chlorocinnamyl | H | OCF₃ | H | H |
| I-142 | 4-fluorocinnamyl | H | OCF₃ | H | H |
| I-143 | 4-bromocinnamyl | H | OCF₃ | H | H |
| I-144 | 4-trifluoromethylcinnamyl | H | OCF₃ | H | H |
| I-145 | 4-trifluoromethoxycinnamyl | H | OCF₃ | H | H |
| I-146 | 4-pentafluoroethoxycinnamyl | H | OCF₃ | H | H |
| I-147 | 4-methoxycinnamyl | H | OCF₃ | H | H |
| I-148 | 4-ethoxycinnamyl | H | OCF₃ | H | H |
| I-149 | 4-cyanocinnamyl | H | OCF₃ | H | H |
| I-150 | 3-(6-chloro-pyridin-3-yl)-allyl | H | OCF₃ | H | H |
| I-151 | 3-(4-chlorophenyl)-but-2-enyl | H | OCF₃ | H | H |
| I-152 | 3-(4-chlorophenyl)-3-fluoro-allyl | H | OCF₃ | H | H |
| I-153 | 3-chloro-4-fluoro-cinnamyl | H | OCF₃ | H | H |
| I-154 | 3,5-dichloro-cinnamyl | H | OCF₃ | H | H |
| I-155 | 5-phenyl-penta-2,4-dienyl | H | OCF₃ | H | H |
| I-156 | 4-isopropyloxycarbonylamino-cinnamyl | H | OCF₃ | H | H |
| I-157 | 3-naphthalen-2-yl-allyl | H | OCF₃ | H | H |
| I-158 | 3-(5-trifluoromethyl-pyridin-2-yl)-allyl | H | OCF₃ | H | H |
| I-159 | 3-(5-chloro-pyridin-2-yl)-allyl | H | OCF₃ | H | H |
| I-160 | 3-pyridin-4-yl-allyl | H | OCF₃ | H | H |
| I-161 | 3-(2-Chloro-pyridin-4-yl)-allyl | H | OCF₃ | H | H |
| I-162 | 4-chlorobenzyl | H | CH₃ | H | H |
| I-163 | Cinnamyl | H | CH₃ | H | H |
| I-164 | 4-chlorocinnamyl | H | CH₃ | H | H |
| I-165 | 4-fluorocinnamyl | H | CH₃ | H | H |
| I-166 | 4-bromocinnamyl | H | CH₃ | H | H |
| I-167 | 4-trifluoromethylcinnamyl | H | CH₃ | H | H |
| I-168 | 4-trifluoromethoxycinnamyl | H | CH₃ | H | H |
| I-169 | 4-pentafluoroethoxycinnamyl | H | CH₃ | H | H |
| I-170 | 4-methoxycinnamyl | H | CH₃ | H | H |
| I-171 | 4-ethoxycinnamyl | H | CH₃ | H | H |
| I-172 | 4-cyanocinnamyl | H | CH₃ | H | H |
| I-173 | 3-(6-chloro-pyridin-3-yl)-allyl | H | CH₃ | H | H |
| I-174 | 3-(4-chlorophenyl)-but-2-enyl | H | CH₃ | H | H |
| I-175 | 3-(4-chlorophenyl)-3-fluoro-allyl | H | CH₃ | H | H |
| I-176 | 3-chloro-4-fluoro-cinnamyl | H | CH₃ | H | H |
| I-177 | 3,5-dichloro-cinnamyl | H | CH₃ | H | H |
| I-178 | 5-phenyl-penta-2,4-dienyl | H | CH₃ | H | H |
| I-179 | 4-isopropyloxycarbonylamino-cinnamyl | H | CH₃ | H | H |
| I-180 | 3-naphthalen-2-yl-allyl | H | CH₃ | H | H |
| I-181 | 3-(5-trifluoromethyl-pyridin-2-yl)-allyl | H | CH₃ | H | H |
| I-182 | 3-(5-chloro-pyridin-2-yl)-allyl | H | CH₃ | H | H |
| I-183 | 3-pyridin-4-yl-allyl | H | CH₃ | H | H |
| I-184 | 3-(2-Chloro-pyridin-4-yl)-allyl | H | CH₃ | H | H |
| I-185 | 4-chlorobenzyl | H | CF₃ | H | H |
| I-186 | Cinnamyl | H | CF₃ | H | H |
| I-187 | 4-chlorocinnamyl | H | CF₃ | H | H |
| I-188 | 4-fluorocinnamyl | H | CF₃ | H | H |
| I-189 | 4-bromocinnamyl | H | CF₃ | H | H |
| I-190 | 4-trifluoromethylcinnamyl | H | CF₃ | H | H |
| I-191 | 4-trifluoromethoxycinnamyl | H | CF₃ | H | H |
| I-192 | 4-pentafluoroethoxycinnamyl | H | CF₃ | H | H |
| I-193 | 4-methoxycinnamyl | H | CF₃ | H | H |
| I-194 | 4-ethoxycinnamyl | H | CF₃ | H | H |
| I-195 | 4-cyanocinnamyl | H | CF₃ | H | H |
| I-196 | 3-(6-chloro-pyridin-3-yl)-allyl | H | CF₃ | H | H |
| I-197 | 3-(4-chlorophenyl)-but-2-enyl | H | CF₃ | H | H |
| I-198 | 3-(4-chlorophenyl)-3-fluoro-allyl | H | CF₃ | H | H |
| I-199 | 3-chloro-4-fluoro-cinnamyl | H | CF₃ | H | H |
| I-200 | 3,5-dichloro-cinnamyl | H | CF₃ | H | H |
| I-201 | 5-phenyl-penta-2,4-dienyl | H | CF₃ | H | H |
| I-202 | 4-isopropyloxycarbonylamino-cinnamyl | H | CF₃ | H | H |
| I-203 | 3-naphthalen-2-yl-allyl | H | CF₃ | H | H |
| I-204 | 3-(5-trifluoromethyl-pyridin-2-yl)-allyl | H | CF₃ | H | H |
| I-205 | 3-(5-chloro-pyridin-2-yl)-allyl | H | CF₃ | H | H |
| I-206 | 3-pyridin-4-yl-allyl | H | CF₃ | H | H |
| I-207 | 3-(2-Chloro-pyridin-4-yl)-allyl | H | CF₃ | H | H |
| I-208 | 4-chlorobenzyl | H | H | Cl | H |
| I-209 | Cinnamyl | H | H | Cl | H |
| I-210 | 4-chlorocinnamyl | H | H | Cl | H |
| I-211 | 4-fluorocinnamyl | H | H | Cl | H |
| I-212 | 4-bromocinnamyl | H | H | Cl | H |
| I-213 | 4-trifluoromethylcinnamyl | H | H | Cl | H |
| I-214 | 4-trifluoromethoxycinnamyl | H | H | Cl | H |
| I-215 | 4-pentafluoroethoxycinnamyl | H | H | Cl | H |
| I-216 | 4-methoxycinnamyl | H | H | Cl | H |
| I-217 | 4-ethoxycinnamyl | H | H | Cl | H |
| I-218 | 4-cyanocinnamyl | H | H | Cl | H |
| I-219 | 3-(6-chloro-pyridin-3-yl)-allyl | H | H | Cl | H |
| I-220 | 3-(4-chlorophenyl)-but-2-enyl | H | H | Cl | H |
| I-221 | 3-(4-chlorophenyl)-3-fluoro-allyl | H | H | Cl | H |
| I-222 | 3-chloro-4-fluoro-cinnamyl | H | H | Cl | H |
| I-223 | 3,5-dichloro-cinnamyl | H | H | Cl | H |
| I-224 | 5-phenyl-penta-2,4-dienyl | H | H | Cl | H |
| I-225 | 4-isopropyloxycarbonylamino-cinnamyl | H | H | Cl | H |
| I-226 | 3-naphthalen-2-yl-allyl | H | H | Cl | H |
| I-227 | 3-(5-trifluoromethyl-pyridin-2-yl)-allyl | H | H | Cl | H |
| I-228 | 3-(5-chloro-pyridin-2-yl)-allyl | H | H | Cl | H |
| I-229 | 3-pyridin-4-yl-allyl | H | H | Cl | H |
| I-230 | 3-(2-Chloro-pyridin-4-yl)-allyl | H | H | Cl | H |
| I-231 | 4-chlorobenzyl | H | H | F | H |
| I-232 | Cinnamyl | H | H | F | H |
| I-233 | 4-chlorocinnamyl | H | H | F | H |
| I-234 | 4-fluorocinnamyl | H | H | F | H |
| I-235 | 4-bromocinnamyl | H | H | F | H |
| I-236 | 4-trifluoromethylcinnamyl | H | H | F | H |
| I-237 | 4-trifluoromethoxycinnamyl | H | H | F | H |
| I-238 | 4-pentafluoroethoxycinnamyl | H | H | F | H |
| I-239 | 4-methoxycinnamyl | H | H | F | H |
| I-240 | 4-ethoxycinnamyl | H | H | F | H |
| I-241 | 4-cyanocinnamyl | H | H | F | H |
| I-242 | 3-(6-chloro-pyridin-3-yl)-allyl | H | H | F | H |
| I-243 | 3-(4-chlorophenyl)-but-2-enyl | H | H | F | H |
| I-244 | 3-(4-chlorophenyl)-3-fluoro-allyl | H | H | F | H |
| I-245 | 3-chloro-4-fluoro-cinnamyl | H | H | F | H |
| I-246 | 3,5-dichloro-cinnamyl | H | H | F | H |
| I-247 | 5-phenyl-penta-2,4-dienyl | H | H | F | H |
| I-248 | 4-isopropyloxycarbonylamino-cinnamyl | H | H | F | H |
| I-249 | 3-naphthalen-2-yl-allyl | H | H | F | H |
| I-250 | 3-(5-trifluoromethyl-pyridin-2-yl)-allyl | H | H | F | H |
| I-251 | 3-(5-chloro-pyridin-2-yl)-allyl | H | H | F | H |
| I-252 | 3-pyridin-4-yl-allyl | H | H | F | H |
| I-253 | 3-(2-Chloro-pyridin-4-yl)-allyl | H | H | F | H |
| I-254 | 4-chlorobenzyl | H | H | Br | H |
| I-255 | Cinnamyl | H | H | Br | H |
| I-256 | 4-chlorocinnamyl | H | H | Br | H |
| I-257 | 4-fluorocinnamyl | H | H | Br | H |
| I-258 | 4-bromocinnamyl | H | H | Br | H |
| I-259 | 4-trifluoromethylcinnamyl | H | H | Br | H |
| I-260 | 4-trifluoromethoxycinnamyl | H | H | Br | H |
| I-261 | 4-pentafluoroethoxycinnamyl | H | H | Br | H |
| I-262 | 4-methoxycinnamyl | H | H | Br | H |
| I-263 | 4-ethoxycinnamyl | H | H | Br | H |
| I-264 | 4-cyanocinnamyl | H | H | Br | H |
| I-265 | 3-(6-chloro-pyridin-3-yl)-allyl | H | H | Br | H |
| I-266 | 3-(4-chlorophenyl)-but-2-enyl | H | H | Br | H |
| I-267 | 3-(4-chlorophenyl)-3-fluoro-allyl | H | H | Br | H |
| I-268 | 3-chloro-4-fluoro-cinnamyl | H | H | Br | H |
| I-269 | 3,5-dichloro-cinnamyl | H | H | Br | H |
| I-270 | 5-phenyl-penta-2,4-dienyl | H | H | Br | H |
| I-271 | 4-isopropyloxycarbonylamino-cinnamyl | H | H | Br | H |
| I-272 | 3-naphthalen-2-yl-allyl | H | H | Br | H |
| I-273 | 3-(5-trifluoromethyl-pyridin-2-yl)-allyl | H | H | Br | H |
| I-274 | 3-(5-chloro-pyridin-2-yl)-allyl | H | H | Br | H |
| I-275 | 3-pyridin-4-yl-allyl | H | H | Br | H |
| I-276 | 3-(2-Chloro-pyridin-4-yl)-allyl | H | H | Br | H |
| I-277 | 4-chlorobenzyl | H | H | OCF₃ | H |
| I-278 | Cinnamyl | H | H | OCF₃ | H |
| I-279 | 4-chlorocinnamyl | H | H | OCF₃ | H |
| I-280 | 4-fluorocinnamyl | H | H | OCF₃ | H |
| I-281 | 4-bromocinnamyl | H | H | OCF₃ | H |
| I-282 | 4-trifluoromethylcinnamyl | H | H | OCF₃ | H |
| I-283 | 4-trifluoromethoxycinnamyl | H | H | OCF₃ | H |
| I-284 | 4-pentafluoroethoxycinnamyl | H | H | OCF₃ | H |
| I-285 | 4-methoxycinnamyl | H | H | OCF₃ | H |
| I-286 | 4-ethoxycinnamyl | H | H | OCF₃ | H |
| I-287 | 4-cyanocinnamyl | H | H | OCF₃ | H |
| I-288 | 3-(6-chloro-pyridin-3-yl)-allyl | H | H | OCF₃ | H |
| I-289 | 3-(4-chlorophenyl)-but-2-enyl | H | H | OCF₃ | H |
| I-290 | 3-(4-chlorophenyl)-3-fluoro-allyl | H | H | OCF₃ | H |
| I-291 | 3-chloro-4-fluoro-cinnamyl | H | H | OCF₃ | H |
| I-292 | 3,5-dichloro-cinnamyl | H | H | OCF₃ | H |
| I-293 | 5-phenyl-penta-2,4-dienyl | H | H | OCF₃ | H |
| I-294 | 4-isopropyloxycarbonylamino-cinnamyl | H | H | OCF₃ | H |
| I-295 | 3-naphthalen-2-yl-allyl | H | H | OCF₃ | H |
| I-296 | 3-(5-trifluoromethyl-pyridin-2-yl)-allyl | H | H | OCF₃ | H |
| I-297 | 3-(5-chloro-pyridin-2-yl)-allyl | H | H | OCF₃ | H |
| I-298 | 3-pyridin-4-yl-allyl | H | H | OCF₃ | H |
| I-299 | 3-(2-Chloro-pyridin-4-yl)-allyl | H | H | OCF₃ | H |
| I-300 | 4-chlorobenzyl | H | H | CH₃ | H |
| I-301 | Cinnamyl | H | H | CH₃ | H |
| I-302 | 4-chlorocinnamyl | H | H | CH₃ | H |
| I-303 | 4-fluorocinnamyl | H | H | CH₃ | H |
| I-304 | 4-bromocinnamyl | H | H | CH₃ | H |
| I-305 | 4-trifluoromethylcinnamyl | H | H | CH₃ | H |
| I-306 | 4-trifluoromethoxycinnamyl | H | H | CH₃ | H |
| I-307 | 4-pentafluoroethoxycinnamyl | H | H | CH₃ | H |
| I-308 | 4-methoxycinnamyl | H | H | CH₃ | H |
| I-309 | 4-ethoxycinnamyl | H | H | CH₃ | H |
| I-310 | 4-cyanocinnamyl | H | H | CH₃ | H |
| I-311 | 3-(6-chloro-pyridin-3-yl)-allyl | H | H | CH₃ | H |
| I-312 | 3-(4-chlorophenyl)-but-2-enyl | H | H | CH₃ | H |
| I-313 | 3-(4-chlorophenyl)-3-fluoro-allyl | H | H | CH₃ | H |
| I-314 | 3-chloro-4-fluoro-cinnamyl | H | H | CH₃ | H |
| I-315 | 3,5-dichloro-cinnamyl | H | H | CH₃ | H |
| I-316 | 5-phenyl-penta-2,4-dienyl | H | H | CH₃ | H |
| I-317 | 4-isopropyloxycarbonylamino-cinnamyl | H | H | CH₃ | H |
| I-318 | 3-naphthalen-2yl-allyl | H | H | CH₃ | H |
| I-319 | 3-(5-trifluoromethyl-pyridin-2-yl)-allyl | H | H | CH₃ | H |
| I-320 | 3-(5-chloro-pyridin-2-yl)-allyl | H | H | CH₃ | H |
| I-321 | 3-pyridin-4-yl-allyl | H | H | CH₃ | H |
| I-322 | 3-(2-Chloro-pyridin-4-yl)-allyl | H | H | CH₃ | H |
| I-323 | 4-chlorobenzyl | H | H | CF₃ | H |
| I-324 | Cinnamyl | H | H | CF₃ | H |
| I-325 | 4-chlorocinnamyl | H | H | CF₃ | H |
| I-326 | 4-fluorocinnamyl | H | H | CF₃ | H |
| I-327 | 4-bromocinnamyl | H | H | CF₃ | H |
| I-328 | 4-trifluoromethylcinnamyl | H | H | CF₃ | H |
| I-329 | 4-trifluoromethoxycinnamyl | H | H | CF₃ | H |
| I-330 | 4-pentafluoroethoxycinnamyl | H | H | CF₃ | H |
| I-331 | 4-methoxycinnamyl | H | H | CF₃ | H |
| I-332 | 4-ethoxycinnamyl | H | H | CF₃ | H |
| I-333 | 4-cyanocinnamyl | H | H | CF₃ | H |
| I-334 | 3-(6-chloro-pyridin-3-yl)-allyl | H | H | CF₃ | H |
| I-335 | 3-(4-chlorophenyl)-but-2-enyl | H | H | CF₃ | H |
| I-336 | 3-(4-chlorophenyl)-3-fluoro-allyl | H | H | CF₃ | H |
| I-337 | 3-chloro-4-fluoro-cinnamyl | H | H | CF₃ | H |
| I-338 | 3,5-dichloro-cinnamyl | H | H | CF₃ | H |
| I-339 | 5-phenyl-penta-2,4-dienyl | H | H | CF₃ | H |
| I-340 | 4-isopropyloxycarbonylamino-cinnamyl | H | H | CF₃ | H |
| I-341 | 3-naphthalen-2-yl-allyl | H | H | CF₃ | H |
| I-342 | 3-(5-trifluoromethyl-pyridin-2-yl)-allyl | H | H | CF₃ | H |
| I-343 | 3-(5-chloro-pyridin-2-yl)-allyl | H | H | CF₃ | H |
| I-344 | 3-pyridin-4-yl-allyl | H | H | CF₃ | H |
| I-345 | 3-(2-Chloro-pyridin-4-yl)-allyl | H | H | CF₃ | H |
| I-346 | 4-chlorobenzyl | F | H | H | H |
| I-347 | Cinnamyl | F | H | H | H |
| I-348 | 4-chlorocinnamyl | F | H | H | H |
| I-349 | 4-fluorocinnamyl | F | H | H | H |
| I-350 | 4-bromocinnamyl | F | H | H | H |
| I-351 | 4-trifluoromethylcinnamyl | F | H | H | H |
| I-352 | 4-trifluoromethoxycinnamyl | F | H | H | H |
| I-353 | 4-pentafluoroethoxycinnamyl | F | H | H | H |
| I-354 | 4-methoxycinnamyl | F | H | H | H |
| I-355 | 4-ethoxycinnamyl | F | H | H | H |
| I-356 | 4-cyanocinnamyl | F | H | H | H |
| I-357 | 3-(6-chloro-pyridin-3-yl)-allyl | F | H | H | H |
| I-358 | 3-(4-chlorophenyl)-but-2-enyl | F | H | H | H |
| I-359 | 3-(4-chlorophenyl)-3-fluoro-allyl | F | H | H | H |
| I-360 | 3-chloro-4-fluoro-cinnamyl | F | H | H | H |
| I-361 | 3,5-dichloro-cinnamyl | F | H | H | H |
| I-362 | 5-phenyl-penta-2,4-dienyl | F | H | H | H |
| I-363 | 4-isopropyloxycarbonylamino-cinnamyl | F | H | H | H |
| I-364 | 3-naphthalen-2-yl-allyl | F | H | H | H |
| I-365 | 3-(5-trifluoromethyl-pyridin-2-yl)-allyl | F | H | H | H |
| I-366 | 3-(5-chloro-pyridin-2-yl)-allyl | F | H | H | H |
| I-367 | 3-pyridin-4-yl-allyl | F | H | H | H |
| I-368 | 3-(2-Chloro-pyridin-4-yl)-allyl | F | H | H | H |
| I-369 | 4-chlorobenzyl | Cl | H | H | H |
| I-370 | Cinnamyl | Cl | H | H | H |
| I-371 | 4-chlorocinnamyl | Cl | H | H | H |
| I-372 | 4-fluorocinnamyl | Cl | H | H | H |
| I-373 | 4-bromocinnamyl | Cl | H | H | H |
| I-374 | 4-trifluoromethylcinnamyl | Cl | H | H | H |
| I-375 | 4-trifluoromethoxycinnamyl | Cl | H | H | H |
| I-376 | 4-pentafluoroethoxycinnamyl | Cl | H | H | H |
| I-377 | 4-methoxycinnamyl | Cl | H | H | H |
| I-378 | 4-ethoxycinnamyl | Cl | H | H | H |
| I-379 | 4-cyanocinnamyl | Cl | H | H | H |
| I-380 | 3-(6-chloro-pyridin-3-yl)-allyl | Cl | H | H | H |
| I-381 | 3-(4-chlorophenyl)-but-2-enyl | Cl | H | H | H |
| I-382 | 3-(4-chlorophenyl)-33-fluoro-allyl | Cl | H | H | H |
| I-383 | 3-chloro-4-fluoro-cinnamyl | Cl | H | H | H |
| I-384 | 3,5-dichloro-cinnamyl | Cl | H | H | H |
| I-385 | 5-phenyl-penta-2,4-dienyl | Cl | H | H | H |
| I-386 | 4-isopropyloxycarbonylamino-cinnamyl | Cl | H | H | H |
| I-387 | 3-naphthalen-2-yl-allyl | Cl | H | H | H |
| I-388 | 3-(5-trifluoromethyl-pyridin-2-yl)-allyl | Cl | H | H | H |
| I-389 | 3-(5-chloro-pyridin-2-yl)-allyl | Cl | H | H | H |
| I-390 | 3-pyridin-4-yl-allyl | Cl | H | H | H |
| I-391 | 3-(2-Chloro-pyridin-4-yl)-allyl | Cl | H | H | H |
| I-392 | 4-chlorobenzyl | Br | H | H | H |
| I-393 | Cinnamyl | Br | H | H | H |
| I-394 | 4-chlorocinnamyl | Br | H | H | H |
| I-395 | 4-fluorocinnamyl | Br | H | H | H |
| I-396 | 4-bromocinnamyl | Br | H | H | H |
| I-397 | 4-trifluoromethylcinnamyl | Br | H | H | H |
| I-398 | 4-trifluoromethoxycinnamyl | Br | H | H | H |
| I-399 | 4-pentafluoroethoxycinnamyl | Br | H | H | H |
| I-400 | 4-methoxycinnamyl | Br | H | H | H |
| I-401 | 4-ethoxycinnamyl | Br | H | H | H |
| I-402 | 4-cyanocinnamyl | Br | H | H | H |
| I-403 | 3-(6-chloro-pyridin-3-yl)-allyl | Br | H | H | H |
| I-404 | 3-(4-chlorophenyl)-but-2-enyl | Br | H | H | H |
| I-405 | 3-(4-chlorophenyl)-3-fluoro-allyl | Br | H | H | H |
| I-406 | 3-chloro-4-fluoro-cinnamyl | Br | H | H | H |
| I-407 | 3,5-dichloro-cinnamyl | Br | H | H | H |
| I-408 | 5-phenyl-penta-2,4-dienyl | Br | H | H | H |
| I-409 | 4-isopropyloxycarbonylamino-cinnamyl | Br | H | H | H |
| I-410 | 3-naphthalen -2-yl-allyl | Br | H | H | H |
| I-411 | 3-(5-trifluoromethyl-pyridin-2-yl)-allyl | Br | H | H | H |
| I-412 | 3-(5-chloro-pyridin-2-yl)-allyl | Br | H | H | H |
| I-413 | 3-pyridin-4-yl-allyl | Br | H | H | H |
| I-414 | 3-(2-Chloro-pyridin-4-yl)-allyl | Br | H | H | H |
| I-415 | 4-chlorobenzyl | CF₃ | H | H | H |
| I-416 | Cinnamyl | CF₃ | H | H | H |
| I-417 | 4-chlorocinnamyl | CF₃ | H | H | H |
| I-418 | 4-fluorocinnamyl | CF₃ | H | H | H |
| I-419 | 4-bromocinnamyl | CF₃ | H | H | H |
| I-420 | 4-trifluoromethylcinnamyl | CF₃ | H | H | H |
| I-421 | 4-trifluoromethoxycinnamyl | CF₃ | H | H | H |
| I-422 | 4-pentafluoroethoxycinnamyl | CF₃ | H | H | H |
| I-423 | 4-methoxycinnamyl | CF₃ | H | H | H |
| I-424 | 4-ethoxycinnamyl | CF₃ | H | H | H |
| I-425 | 4-cyanocinnamyl | CF₃ | H | H | H |
| I-426 | 3-(6-chloro-pyridin-3-yl)-allyl | CF₃ | H | H | H |
| I-427 | 3-(4-chlorophenyl)-but-2-enyl | CF₃ | H | H | H |
| I-428 | 3-(4-chlorophenyl)-3-fluoro-allyl | CF₃ | H | H | H |
| I-429 | 3-chloro-4-fluoro-cinnamyl | CF₃ | H | H | H |
| I-430 | 3,5-dichloro-cinnamyl | CF₃ | H | H | H |
| I-431 | 5-phenyl-penta-2,4-dienyl | CF₃ | H | H | H |
| I-432 | 4-isopropyloxycarbonylamino-cinnamyl | CF₃ | H | H | H |
| I-433 | 3-naphthalen-2-yl-allyl | CF₃ | H | H | H |
| I-434 | 3-(5-trifluoromethyl-pyridin-2-yl)-allyl | CF₃ | H | H | H |
| I-435 | 3-(5-chloro-pyridin-2-yl)-allyl | CF₃ | H | H | H |
| I-436 | 3-pyridin-4-yl-allyl | CF₃ | H | H | H |
| I-437 | 3-(2-Chloro-pyridin-4-yl)-allyl | CF₃ | H | H | H |
| I-438 | 4-chlorobenzyl | H | H | H | F |
| I-439 | Cinnamyl | H | H | H | F |
| I-440 | 4-chlorocinnamyl | H | H | H | F |
| I-441 | 4-fluorocinnamyl | H | H | H | F |
| I-442 | 4-bromocinnamyl | H | H | H | F |
| I-443 | 4-trifluoromethylcinnamyl | H | H | H | F |
| I-444 | 4-trifluoromethoxycinnamyl | H | H | H | F |
| I-445 | 4-pentafluoroethoxycinnamyl | H | H | H | F |
| I-446 | 4-methoxycinnamyl | H | H | H | F |
| I-447 | 4-ethoxycinnamyl | H | H | H | F |
| I-448 | 4-cyanocinnamyl | H | H | H | F |
| I-449 | 3-(6-chloro-pyridin-3-yl)-allyl | H | H | H | F |
| I-450 | 3-(4-chlorophenyl)-but-2-enyl | H | H | H | F |
| I-451 | 3-(4-chlorophenyl)-3-fluoro-allyl | H | H | H | F |
| I-452 | 3-chloro-4-fluoro-cinnamyl | H | H | H | F |
| I-453 | 3,5-dichloro-cinnamyl | H | H | H | F |
| I-454 | 5-phenyl-penta-2,4-dienyl | H | H | H | F |
| I-455 | 4-isopropyloxycarbonylamino-cinnamyl | H | H | H | F |
| I-456 | 3-naphthalen-2-yl-allyl | H | H | H | F |
| I-457 | 3-(5-trifluoromethyl-pyridin-2-yl)-allyl | H | H | H | F |
| I-458 | 3-(5-chloro-pyridin-2-yl)-allyl | H | H | H | F |
| I-459 | 3-pyridin-4-yl-allyl | H | H | H | F |
| I-460 | 3-(2-Chloro-pyridin-4-yl)-allyl | H | H | H | F |
| I-461 | 4-chlorobenzyl | H | H | H | Cl |
| I-462 | Cinnamyl | H | H | H | Cl |
| I-463 | 4-chlorocinnamyl | H | H | H | Cl |
| I-464 | 4-fluorocinnamyl | H | H | H | Cl |
| I-465 | 4-bromocinnamyl | H | H | H | Cl |
| I-466 | 4-trifluoromethylcinnamyl | H | H | H | Cl |
| I-467 | 4-trifluoromethoxycinnamyl | H | H | H | Cl |
| I-468 | 4-pentafluoroethoxycinnamyl | H | H | H | Cl |
| I-469 | 4-methoxycinnamyl | H | H | H | Cl |
| I-470 | 4-ethoxycinnamyl | H | H | H | Cl |
| I-471 | 4-cyanocinnamyl | H | H | H | Cl |
| I-472 | 3-(6-chloro-pyridin-3-yl)-allyl | H | H | H | Cl |
| I-473 | 3-(4-chlorophenyl)-but-2-enyl | H | H | H | Cl |
| I-474 | 3-(4-chlorophenyl)-3-fluoro-allyl | H | H | H | Cl |
| I-475 | 3-chloro-4-fluoro-cinnamyl | H | H | H | Cl |
| I-476 | 3,5-dichloro-cinnamyl | H | H | H | Cl |
| I-477 | 5-phenyl-penta-2,4-dienyl | H | H | H | Cl |
| I-478 | 4-isopropyloxycarbonylamino-cinnamyl | H | H | H | Cl |
| I-479 | 3-naphthalen-2-yl-allyl | H | H | H | Cl |
| I-480 | 3-(5-trifluoromethyl-pyridin-2-yl)-allyl | H | H | H | Cl |
| I-481 | 3-(5-chloro-pyridin-2-yl)-allyl | H | H | H | Cl |
| I-482 | 3-pyridin-4-yl-allyl | H | H | H | Cl |
| I-483 | 3-(2-Chloro-pyridin-4-yl)-allyl | H | H | H | Cl |
| I-484 | 4-chlorobenzyl | H | F | F | H |
| I-485 | Cinnamyl | H | F | F | H |
| I-486 | 4-chlorocinnamyl | H | F | F | H |
| I-487 | 4-fluorocinnamyl | H | F | F | H |
| I-488 | 4-bromocinnamyl | H | F | F | H |
| I-489 | 4-trifluoromethylcinnamyl | H | F | F | H |
| I-490 | 4-trifluoromethoxycinnamyl | H | F | F | H |
| I-491 | 4-pentafluoroethoxycinnamyl | H | F | F | H |
| I-492 | 4-methoxycinnamyl | H | F | F | H |
| I-493 | 4-ethoxycinnamyl | H | F | F | H |
| I-494 | 4-cyanocinnamyl | H | F | F | H |
| I-495 | 3-(6-chloro-pyridin-3-yl)-allyl | H | F | F | H |
| I-496 | 3-(4-chlorophenyl)-but-2-enyl | H | F | F | H |
| I-497 | 3-(4-chlorophenyl)-3-fluoro-allyl | H | F | F | H |
| I-498 | 3-chloro-4-fluoro-cinnamyl | H | F | F | H |
| I-499 | 3,5-dichloro-cinnamyl | H | F | F | H |
| I-500 | 5-phenyl-penta-2,4-dienyl | H | F | F | H |
| I-501 | 4-isopropyloxycarbonylamino-cinnamyl | H | F | F | H |
| I-502 | 3-naphthalen-2-yl-allyl | H | F | F | H |
| I-503 | 3-(5-trifluoromethyl-pyridin-2-yl)-allyl | H | F | F | H |
| I-504 | 3-(5-chloro-pyridin-2-yl)-allyl | H | F | F | H |
| I-505 | 3-pyridin-4-yl-allyl | H | F | F | H |
| I-506 | 3-(2-Chloro-pyridin-4-yl)-allyl | H | F | F | H |
| I-507 | 4-chlorobenzyl | H | F | Cl | H |
| I-508 | Cinnamyl | H | F | Cl | H |
| I-509 | 4-chlorocinnamyl | H | F | Cl | H |
| I-510 | 4-fluorocinnamyl | H | F | Cl | H |
| I-511 | 4-bromocinnamyl | H | F | Cl | H |
| I-512 | 4-trifluoromethylcinnamyl | H | F | Cl | H |
| I-513 | 4-trifluoromethoxycinnamyl | H | F | Cl | H |
| I-514 | 4-pentafluoroethoxycinnamyl | H | F | Cl | H |
| I-515 | 4-methoxycinnamyl | H | F | Cl | H |
| I-516 | 4-ethoxycinnamyl | H | F | Cl | H |
| I-517 | 4-cyanocinnamyl | H | F | Cl | H |
| I-518 | 3-(6-chloro-pyridin-3-yl)-allyl | H | F | Cl | H |
| I-519 | 3-(4-chlorophenyl)-but-2-enyl | H | F | Cl | H |
| I-520 | 3-(4-chlorophenyl)-3-fluoro-allyl | H | F | Cl | H |
| I-521 | 3-chloro-4-fluoro-cinnamyl | H | F | Cl | H |
| I-522 | 3,5-dichloro-cinnamyl | H | F | Cl | H |
| I-523 | 5-phenyl-penta-2,4-dienyl | H | F | Cl | H |
| I-524 | 4-isopropyloxycarbonylamino-cinnamyl | H | F | Cl | H |
| I-525 | 3-naphthalen-2-yl-allyl | H | F | Cl | H |
| I-526 | 3-(5-trifluoromethyl-pyridin-2-yl)-allyl | H | F | Cl | H |
| I-527 | 3-(5-chloro-pyridin-2-yl)-allyl | H | F | Cl | H |
| I-528 | 3-pyridin-4-yl-allyl | H | F | Cl | H |
| I-529 | 3-(2-Chloro-pyridin-4-yl)-allyl | H | F | Cl | H |
| I-530 | 4-chlorobenzyl | H | Cl | F | H |
| I-531 | Cinnamyl | H | Cl | F | H |
| I-532 | 4-chlorocinnamyl | H | Cl | F | H |
| I-533 | 4-fluorocinnamyl | H | Cl | F | H |
| I-534 | 4-bromocinnamyl | H | Cl | F | H |
| I-535 | 4-trifluoromethylcinnamyl | H | Cl | F | H |
| I-536 | 4-trifluoromethoxycinnamyl | H | Cl | F | H |
| I-537 | 4-pentafluoroethoxycinnamyl | H | Cl | F | H |
| I-538 | 4-methoxycinnamyl | H | Cl | F | H |
| I-539 | 4-ethoxycinnamyl | H | Cl | F | H |
| I-540 | 4-cyanocinnamyl | H | Cl | F | H |
| I-541 | 3-(6-chloro-pyridin-3-yl)-allyl | H | Cl | F | H |
| I-542 | 3-(4-chlorophenyl)-but-2-enyl | H | Cl | F | H |
| I-543 | 3-(4-chlorophenyl)-3-fluoro-allyl | H | Cl | F | H |
| I-544 | 3-chloro-4-fluoro-cinnamyl | H | Cl | F | H |
| I-545 | 3,5-dichloro-cinnamyl | H | Cl | F | H |
| I-546 | 5-phenyl-penta-2,4-dienyl | H | Cl | F | H |
| I-547 | 4-isopropyloxycarbonylamino-cinnamyl | H | Cl | F | H |
| I-548 | 3-naphthalen-2-yl-allyl | H | Cl | F | H |
| I-549 | 3-(5-trifluoromethyl-pyridin-2-yl)-allyl | H | Cl | F | H |
| I-550 | 3-(5-chloro-pyridin-2-yl)-allyl | H | Cl | F | H |
| I-551 | 3-pyridin-4-yl-allyl | H | Cl | F | H |
| I-552 | 3-(2-Chloro-pyridin-4-yl)-allyl | H | Cl | F | H |
| I-553 | 4-chlorobenzyl | H | Cl | Cl | H |
| I-554 | Cinnamyl | H | Cl | Cl | H |
| I-555 | 4-chlorocinnamyl | H | Cl | Cl | H |
| I-556 | 4-fluorocinnamyl | H | Cl | Cl | H |
| I-557 | 4-bromocinnamyl | H | Cl | Cl | H |
| I-558 | 4-trifluoromethylcinnamyl | H | Cl | Cl | H |
| I-559 | 4-trifluoromethoxycinnamyl | H | Cl | Cl | H |
| I-560 | 4-pentafluoroethoxycinnamyl | H | Cl | Cl | H |
| I-561 | 4-methoxycinnamyl | H | Cl | Cl | H |
| I-562 | 4-ethoxycinnamyl | H | Cl | Cl | H |
| I-563 | 4-cyanocinnamyl | H | Cl | Cl | H |
| I-564 | 3-(6-chloro-pyridin-3-yl)-allyl | H | Cl | Cl | H |
| I-565 | 3-(4-chlorophenyl)-but-2-enyl | H | Cl | Cl | H |
| I-566 | 3-(4-chlorophenyl)-3-fluoro-allyl | H | Cl | Cl | H |
| I-567 | 3-chloro-4-fluoro-cinnamyl | H | Cl | Cl | H |
| I-568 | 3,5-dichloro-cinnamyl | H | Cl | Cl | H |
| I-569 | 5-phenyl-penta-2,4-dienyl | H | Cl | Cl | H |
| I-570 | 4-isopropyloxycarbonylamino-cinnamyl | H | Cl | Cl | H |
| I-571 | 3-naphthalen-2-yl-allyl | H | Cl | Cl | H |
| I-572 | 3-(5-trifluoromethyl-pyridin-2-yl)-allyl | H | Cl | Cl | H |
| I-573 | 3-(5-chloro-pyridin-2-yl)-allyl | H | Cl | Cl | H |
| I-574 | 3-pyridin-4-yl-allyl | H | Cl | Cl | H |
| I-575 | 3-(2-Chloro-pyridin-4-yl)-allyl | H | Cl | Cl | H |
| I-576 | 4-chlorobenzyl | H | —OCF₂O— | | H |
| I-577 | Cinnamyl | H | —OCF₂O— | | H |
| I-578 | 4-chlorocinnamyl | H | —OCF₂O— | | H |
| I-579 | 4-fluorocinnamyl | H | —OCF₂O— | | H |
| I-580 | 4-bromocinnamyl | H | —OCF₂O— | | H |
| I-581 | 4-trifluoromethylcinnamyl | H | —OCF₂O— | | H |
| I-582 | 4-trifluoromethoxycinnamyl | H | —OCF₂O— | | H |
| I-583 | 4-pentafluoroethoxycinnamyl | H | —OCF₂O— | | H |
| I-584 | 4-methoxycinnamyl | H | —OCF₂O— | | H |
| I-585 | 4-ethoxycinnamyl | H | —OCF₂O— | | H |
| I-586 | 4-cyanocinnamyl | H | —OCF₂O— | | H |
| I-587 | 3-(6-chloro-pyridin-3-yl)-allyl | H | —OCF₂O— | | H |
| I-588 | 3-(4-chlorophenyl)-but-2-enyl | H | —OCF₂O— | | H |
| I-589 | 3-(4-chlorophenyl)-3-fluoro-allyl | H | —OCF₂O— | | H |
| I-590 | 3-chloro-4-fluoro-cinnamyl | H | —OCF₂O— | | H |
| I-591 | 3,5-dichloro-cinnamyl | H | —OCF₂O— | | H |
| I-592 | 5-phenyl-penta-2,4-dienyl | H | —OCF₂O— | | H |
| I-593 | 4-isopropyloxycarbonylamino-cinnamyl | H | —OCF₂O— | | H |
| I-594 | 3-naphthalen-2-yl-allyl | H | —OCF₂O— | | H |
| I-595 | 3-(5-trifluoromethyl-pyridin-2-yl)-allyl | H | —OCF₂O— | | H |
| I-596 | 3-(5-chloro-pyridin-2-yl)-allyl | H | —OCF₂O— | | H |
| I-597 | 3-pyridin-4-yl-allyl | H | —OCF₂O— | | H |
| I-598 | 3-(2-Chloro-pyridin-4-yl)-allyl | H | —OCF₂O— | | H |
| I-599 | 4-chlorobenzyl | H | —C₄H₄— | | H |
| I-600 | Cinnamyl | H | —C₄H₄— | | H |
| I-601 | 4-chlorocinnamyl | H | —C₄H₄— | | H |
| I-602 | 4-fluorocinnamyl | H | —C₄H₄— | | H |
| I-603 | 4-bromocinnamyl | H | —C₄H₄— | | H |
| I-604 | 4-trifluoromethylcinnamyl | H | —C₄H₄— | | H |
| I-605 | 4-trifluoromethoxycinnamyl | H | —C₄H₄— | | H |
| I-606 | 4-pentafluoroethoxycinnamyl | H | —C₄H₄— | | H |
| I-607 | 4-methoxycinnamyl | H | —C₄H₄— | | H |
| I-608 | 4-ethoxycinnamyl | H | —C₄H₄— | | H |
| I-609 | 4-cyanocinnamyl | H | —C₄H₄— | | H |
| I-610 | 3-(6-chloro-pyridin-3-yl)-allyl | H | —C₄H₄— | | H |
| I-611 | 3-(4-chlorophenyl)-but-2-enyl | H | —C₄H₄— | | H |
| I-612 | 3-(4-chlorophenyl)-3-fluoro-allyl | H | —C₄H₄— | | H |
| I-613 | 3-chloro-4-fluoro-cinnamyl | H | —C₄H₄— | | H |
| I-614 | 3,5-dichloro-cinnamyl | H | —C₄H₄— | | H |
| I-615 | 5-phenyl-penta-2,4-dienyl | H | —C₄H₄— | | H |
| I-616 | 4-isopropyloxycarbonylamino-cinnamyl | H | —C₄H₄— | | H |
| I-617 | 3-naphthalen-2-yl-allyl | H | —C₄H₄— | | H |
| I-618 | 3-(5-trifluoromethyl-pyridin-2-yl)-allyl | H | —C₄H₄— | | H |
| I-619 | 3-(5-chloro-pyridin-2-yl)-allyl | H | —C₄H₄— | | H |
| I-620 | 3-pyridin-4-yl-allyl | H | —C₄H₄— | | H |
| I-621 | 3-(2-Chloro-pyridin-4-yl)-allyl | H | —C₄H₄— | | H |
| I-622 | 4-chlorobenzyl | Cl | H | Cl | H |
| I-623 | Cinnamyl | Cl | H | Cl | H |
| I-624 | 4-chlorocinnamyl | Cl | H | Cl | H |
| I-625 | 4-fluorocinnamyl | Cl | H | Cl | H |
| I-226 | 4-bromocinnamyl | Cl | H | Cl | H |
| I-627 | 4-trifluoromethylcinnamyl | Cl | H | Cl | H |
| I-628 | 4-trifluoromethoxycinnamyl | Cl | H | Cl | H |
| I-629 | 4-pentafluoroethoxycinnamyl | Cl | H | Cl | H |
| I-630 | 4-methoxycinnamyl | Cl | H | Cl | H |
| I-631 | 4-ethoxycinnamyl | Cl | H | Cl | H |
| I-632 | 4-cyanocinnamyl | Cl | H | Cl | H |
| I-633 | 3-(6-chloro-pyridin-3-yl)-allyl | Cl | H | Cl | H |
| I-634 | 3-(4-chlorophenyl)-but-2-enyl | Cl | H | Cl | H |
| I-635 | 3-(4-chlorophenyl)-3-fluoro-allyl | Cl | H | Cl | H |
| I-636 | 3-chloro-4-fluoro-cinnamyl | Cl | H | Cl | H |
| I-637 | 3,5-dichloro-cinnamyl | Cl | H | Cl | H |
| I-638 | 5-phenyl-penta-2,4-dienyl | Cl | H | Cl | H |
| I-639 | 4-isopropyloxycarbonylamino-cinnamyl | Cl | H | Cl | H |
| I-640 | 3-naphthalen-2-yl-allyl | Cl | H | Cl | H |
| I-641 | 3-(5-trifluoromethyl-pyridin-2-yl)-allyl | Cl | H | Cl | H |
| I-642 | 3-(5-chloro-pyridin-2-yl)-allyl | Cl | H | Cl | H |
| I-643 | 3-pyridin-4-yl-allyl | Cl | H | Cl | H |
| I-644 | 3-(2-Chloro-pyridin-4-yl)-allyl | Cl | H | Cl | H |
| I-645 | 4-chlorobenzyl | Cl | Cl | H | H |
| I-646 | Cinnamyl | Cl | Cl | H | H |
| I-647 | 4-chlorocinnamyl | Cl | Cl | H | H |
| I-648 | 4-fluorocinnamyl | Cl | Cl | H | H |
| I-649 | 4-bromocinnamyl | Cl | Cl | H | H |
| I-650 | 4-trifluoromethylcinnamyl | Cl | Cl | H | H |
| I-651 | 4-trifluoromethoxycinnamyl | Cl | Cl | H | H |
| I-652 | 4-pentafluoroethoxycinnamyl | Cl | Cl | H | H |
| I-653 | 4-methoxycinnamyl | Cl | Cl | H | H |
| I-654 | 4-ethoxycinnamyl | Cl | Cl | H | H |
| I-655 | 4-cyanocinnamyl | Cl | Cl | H | H |
| I-656 | 3-(6-chloro-pyridin-3-yl)-allyl | Cl | Cl | H | H |
| I-657 | 3-(4-chlorophenyl)-but-2-enyl | Cl | Cl | H | H |
| I-658 | 3-(4-chlorophenyl)-3-fluoro-allyl | Cl | Cl | H | H |
| I-659 | 3-chloro-4-fluoro-cinnamyl | Cl | Cl | H | H |
| I-660 | 3,5-dichloro-cinnamyl | Cl | Cl | H | H |
| I-661 | 5-phenyl-penta-2,4-dienyl | Cl | Cl | H | H |
| I-662 | 4-isopropyloxycarbonylamino-cinnamyl | Cl | Cl | H | H |
| I-663 | 3-naphthalen-2-yl-allyl | Cl | Cl | H | H |
| I-664 | 3-(5-trifluoromethyl-pyridin-2-yl)-allyl | Cl | Cl | H | H |
| I-665 | 3-(5-chloro-pyridin-2-yl)-allyl | Cl | Cl | H | H |
| I-666 | 3-pyridin-4-yl-allyl | Cl | Cl | H | H |
| I-667 | 3-(2-Chloro-pyridin-4-yl)-allyl | Cl | Cl | H | H |
| I-668 | 4-chlorobenzyl | H | Cl | H | Cl |
| I-669 | Cinnamyl | H | Cl | H | Cl |
| I-670 | 4-chlorocinnamyl | H | Cl | H | Cl |
| I-671 | 4-fluorocinnamyl | H | Cl | H | Cl |
| I-672 | 4-bromocinnamyl | H | Cl | H | Cl |
| I-673 | 4-trifluoromethylcinnamyl | H | Cl | H | Cl |
| I-674 | 4-trifluoromethoxycinnamyl | H | Cl | H | Cl |
| I-675 | 4-pentafluoroethoxycinnamyl | H | Cl | H | Cl |
| I-676 | 4-methoxycinnamyl | H | Cl | H | Cl |
| I-677 | 4-ethoxycinnamyl | H | Cl | H | Cl |
| I-678 | 4-cyanocinnamyl | H | Cl | H | Cl |
| I-679 | 3-(6-chloro-pyridin-3-yl)-allyl | H | Cl | H | Cl |
| I-680 | 3-(4-chlorophenyl)-but-2-enyl | H | Cl | H | Cl |
| I-681 | 3-(4-chlorophenyl)-3-fluoro-allyl | H | Cl | H | Cl |
| I-682 | 3-chloro-4-fluoro-cinnamyl | H | Cl | H | Cl |
| I-683 | 3,5-dichloro-cinnamyl | H | Cl | H | Cl |
| I-684 | 5-phenyl-penta-2,4-dienyl | H | Cl | H | Cl |
| I-685 | 4-isopropyloxycarbonylamino-cinnamyl | H | Cl | H | Cl |
| I-686 | 3-naphthalen-2-yl-allyl | H | Cl | H | Cl |
| I-687 | 3-(5-trifluoromethyl-pyridin-2-yl)-allyl | H | Cl | H | Cl |
| I-688 | 3-(5-chloro-pyridin-2-yl)-allyl | H | Cl | H | Cl |
| I-689 | 3-pyridin-4-yl-allyl | H | Cl | H | Cl |
| I-690 | 3-(2-Chloro-pyridin-4-yl)-allyl | H | Cl | H | Cl |
| I-691 | 4-chlorobenzyl | H | F | H | F |
| I-692 | Cinnamyl | H | F | H | F |
| I-693 | 4-chlorocinnamyl | H | F | H | F |
| I-694 | 4-fluorocinnamyl | H | F | H | F |
| I-695 | 4-bromocinnamyl | H | F | H | F |
| I-696 | 4-trifluoromethylcinnamyl | H | F | H | F |
| I-697 | 4-trifluoromethoxycinnamyl | H | F | H | F |
| I-698 | 4-pentafluoroethoxycinnamyl | H | F | H | F |
| I-699 | 4-methoxycinnamyl | H | F | H | F |
| I-700 | 4-ethoxycinnamyl | H | F | H | F |
| I-701 | 4-cyanocinnamyl | H | F | H | F |
| I-702 | 3-(6-chloro-pyridin-3-yl)-allyl | H | F | H | F |
| I-703 | 3-(4-chlorophenyl)-but-2-enyl | H | F | H | F |
| I-704 | 3-(4-chlorophenyl)-3-fluoro-allyl | H | F | H | F |
| I-705 | 3-chloro-4-fluoro-cinnamyl | H | F | H | F |
| I-706 | 3,5-dichloro-cinnamyl | H | F | H | F |
| I-707 | 5-phenyl-penta-2,4-dienyl | H | F | H | F |
| I-708 | 4-isopropyloxycarbonylamino-cinnamyl | H | F | H | F |
| I-709 | 3-naphthalen-2-yl-allyl | H | F | H | F |
| I-710 | 3-(5-trifluoromethyl-pyridin-2-yl)-allyl | H | F | H | F |
| I-711 | 3-(5-chloro-pyridin-2-yl)-allyl | H | F | H | F |
| I-712 | 3-pyridin-4-yl-allyl | H | F | H | F |
| I-713 | 3-(2-Chloro-pyridin-4-yl)-allyl | H | F | H | F |
| I-714 | 4-chlorobenzyl | F | H | F | H |
| I-715 | Cinnamyl | F | H | F | H |
| I-716 | 4-chlorocinnamyl | F | H | F | H |
| I-717 | 4-fluorocinnamyl | F | H | F | H |
| I-718 | 4-bromocinnamyl | F | H | F | H |
| I-719 | 4-trifluoromethylcinnamyl | F | H | F | H |
| I-720 | 4-trifluoromethoxycinnamyl | F | H | F | H |
| I-721 | 4-pentafluoroethoxycinnamyl | F | H | F | H |
| I-722 | 4-methoxycinnamyl | F | H | F | H |
| I-723 | 4-ethoxycinnamyl | F | H | F | H |
| I-724 | 4-cyanocinnamyl | F | H | F | H |
| I-725 | 3-(6-chloro-pyridin-3-yl)-allyl | F | H | F | H |
| I-726 | 3-(4-chlorophenyl)-but-2-enyl | F | H | F | H |
| I-727 | 3-(4-chlorophenyl)-3-fluoro-allyl | F | H | F | H |
| I-728 | 3-chloro-4-fluoro-cinnamyl | F | H | F | H |
| I-729 | 3,5-dichloro-cinnamyl | F | H | F | H |
| I-730 | 5-phenyl-penta-2,4-dienyl | F | H | F | H |
| I-731 | 4-isopropyloxycarbonylamino-cinnamyl | F | H | F | H |
| I-732 | 3-naphthalen-2-yl-allyl | F | H | F | H |
| I-733 | 3-(5-trifluoromethyl-pyridin-2-yl)-allyl | F | H | F | H |
| I-734 | 3-(5-chloro-pyridin-2-yl)-allyl | F | H | F | H |
| I-735 | 3-pyridin-4-yl-allyl | F | H | F | H |
| I-736 | 3-(2-Chloro-pyridin-4-yl)-allyl | F | H | F | H |
| I-737 | 4-chlorobenzyl | F | F | H | H |
| I-738 | Cinnamyl | F | F | H | H |
| I-739 | 4-chlorocinnamyl | F | F | H | H |
| I-740 | 4-fluorocinnamyl | F | F | H | H |
| I-741 | 4-bromocinnamyl | F | F | H | H |
| I-742 | 4-trifluoromethylcinnamyl | F | F | H | H |
| I-743 | 4-trifluoromethoxycinnamyl | F | F | H | H |
| I-744 | 4-pentafluoroethoxycinnamyl | F | F | H | H |
| I-745 | 4-methoxycinnamyl | F | F | H | H |
| I-746 | 4-ethoxycinnamyl | F | F | H | H |
| I-747 | 4-cyanocinnamyl | F | F | H | H |
| I-748 | 3-(6-chloro-pyridin-3-yl)-allyl | F | F | H | H |
| I-749 | 3-(4-chlorophenyl)-but-2-enyl | F | F | H | H |
| I-750 | 3-(4-chlorophenyl)-3-fluoro-allyl | F | F | H | H |
| I-751 | 3-chloro-4-fluoro-cinnamyl | F | F | H | H |
| I-752 | 3,5-dichloro-cinnamyl | F | F | H | H |
| I-753 | 5-phenyl-penta-2,4-dienyl | F | F | H | H |
| I-754 | 4-isopropyloxycarbonylamino-cinnamyl | F | F | H | H |
| I-755 | 3-naphthalen-2-yl-allyl | F | F | H | H |
| I-756 | 3-(5-trifluoromethyl-pyridin-2-yl)-allyl | F | F | H | H |
| I-757 | 3-(5-chloro-pyridin-2-yl)-allyl | F | F | H | H |
| I-758 | 3-pyridin-4-yl-allyl | F | F | H | H |
| I-759 | 3-(2-Chloro-pyridin-4-yl)-allyl | F | F | H | H |
| I-760 | 4-chlorobenzyl | Cl | F | H | H |
| I-761 | Cinnamyl | Cl | F | H | H |
| I-762 | 4-chlorocinnamyl | Cl | F | H | H |
| I-763 | 4-fluorocinnamyl | Cl | F | H | H |
| I-764 | 4-bromocinnamyl | Cl | F | H | H |
| I-765 | 4-trifluoromethylcinnamyl | Cl | F | H | H |
| I-766 | 4-trifluoromethoxycinnamyl | Cl | F | H | H |
| I-767 | 4-pentafluoroethoxycinnamyl | Cl | F | H | H |
| I-768 | 4-methoxycinnamyl | Cl | F | H | H |
| I-769 | 4-ethoxycinnamyl | Cl | F | H | H |
| I-770 | 4-cyanocinnamyl | Cl | F | H | H |
| I-771 | 3-(6-chloro-pyridin-3-yl)-allyl | Cl | F | H | H |
| I-772 | 3-(4-chlorophenyl)-but-2-enyl | Cl | F | H | H |
| I-773 | 3-(4-chlorophenyl)-3-fluoro-allyl | Cl | F | H | H |
| I-774 | 3-chloro-4-fluoro-cinnamyl | Cl | F | H | H |
| I-775 | 3,5-dichloro-cinnamyl | Cl | F | H | H |
| I-776 | 5-phenyl-penta-2,4-dienyl | Cl | F | H | H |
| I-777 | 4-isopropyloxycarbonylamino-cinnamyl | Cl | F | H | H |
| I-778 | 3-naphthalen-2-yl-allyl | Cl | F | H | H |
| I-779 | 3-(5-trifluoromethyl-pyridin-2-yl)-allyl | Cl | F | H | H |
| I-780 | 3-(5-chloro-pyridin-2-yl)-allyl | Cl | F | H | H |
| I-781 | 3-pyridin-4-yl-allyl | Cl | F | H | H |
| I-782 | 3-(2-Chloro-pyridin-4-yl)-allyl | Cl | F | H | H |

Table II provides 782 compounds of formula Ib 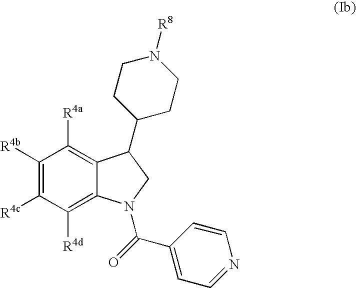
wherein the values of R⁸, R^{4a}, R^{4b}, R^{4c} and R^{4d} are given in Table 1

Table III provides 782 compounds of formula Ic 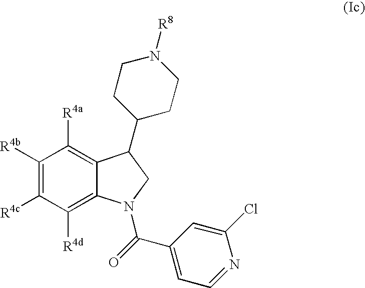
wherein the values of R⁸, R^{4a}, R^{4b}, R^{4c} and R^{4d} are given in Table 1

Table IV provides 782 compounds of formula Id 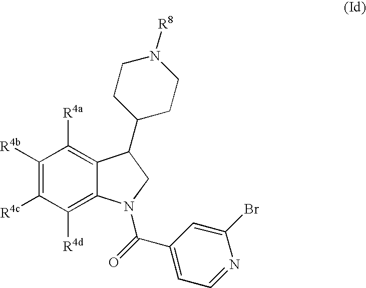
wherein the values of R⁸, R^{4a}, R^{4b}, R^{4c} and R^{4d} are given in Table 1

Table V provides 782 compounds of formula Ie 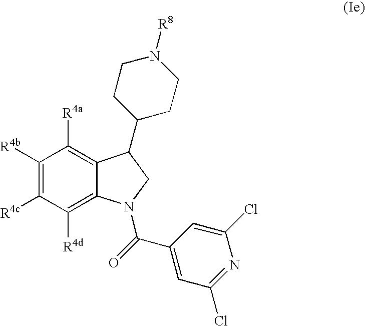
wherein the values of R⁸, R^{4a}, R^{4b}, R^{4c} and R^{4d} are given in Table 1

Table VI provides 782 compounds of formula If 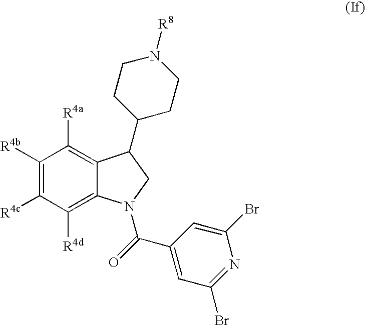
wherein the values of R⁸, R^{4a}, R^{4b}, R^{4c} and R^{4d} are given in Table 1

Table VII provides 782 compounds of formula Ig 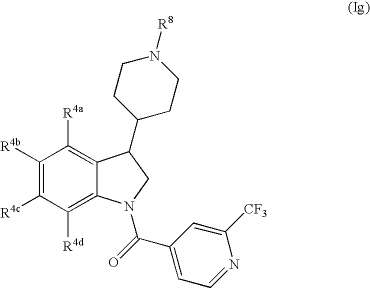
wherein the values of R⁸, R^{4a}, R^{4b}, R^{4c} and R^{4d} are given in Table 1

Table VIII provides 782 compounds of formula Ih 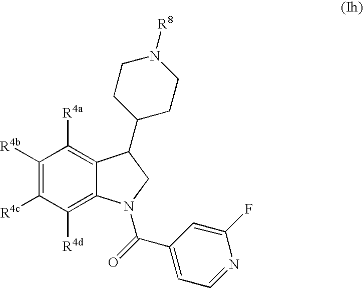
wherein the values of R⁸, R^{4a}, R^{4b}, R^{4c} and R^{4d} are given in Table 1

Table IX provides 782 compounds of formula Ii 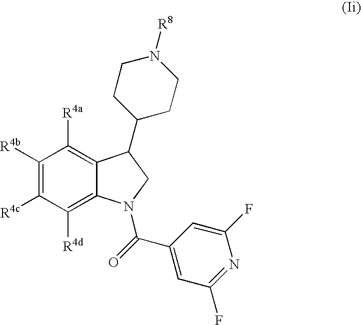
wherein the values of R⁸, R^{4a}, R^{4b}, R^{4c} and R^{4d} are given in Table 1

Table X provides 782 compounds of formula Ij 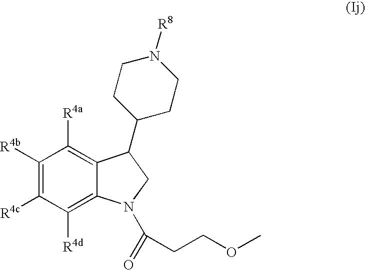
wherein the values of R⁸, R^{4a}, R^{4b}, R^{4c} and R^{4d} are given in Table 1

Table XI provides 782 compounds of formula Ik 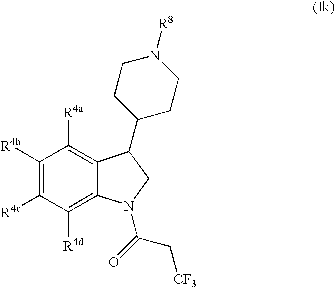
wherein the values of R⁸, R^{4a}, R^{4b}, R^{4c} and R^{4d} are given in Table 1

Table XII provides 782 compounds of formula Il 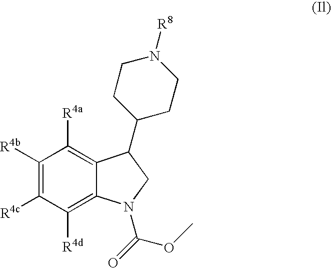
wherein the values of R⁸, R^{4a}, R^{4b}, R^{4c} and R^{4d} are given in Table 1

Table XIII provides 782 compounds of formula Im 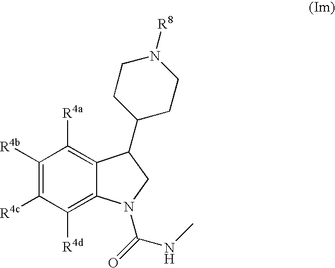
wherein the values of R⁸, R^{4a}, R^{4b}, R^{4c} and R^{4d} are given in Table 1

Table X)V provides 782 compounds of formula In 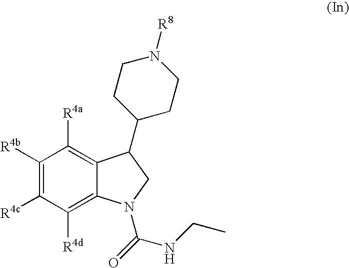
wherein the values of R⁸, R^{4a}, R^{4b}, R^{4c} and R^{4d} are given in Table 1

Table XV provides 782 compounds of formula lo 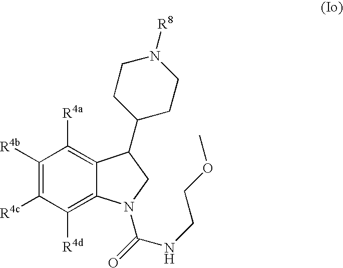
wherein the values of R⁸, R^{4a}, R^{4b}, R^{4c} and R^{4d} are given in Table 1

Table XVI provides 782 compounds of formula Ip 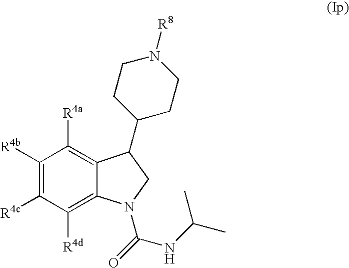
wherein the values of R⁸, R^{4a}, R^{4b}, R^{4c} and R^{4d} are given in Table 1

Table XVII provides 782 compounds of formula Iq 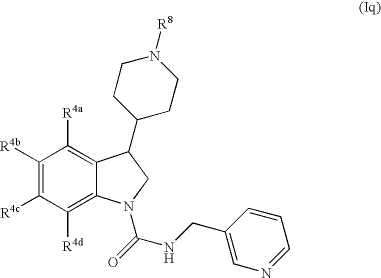
wherein the values of R⁸, R^{4a}, R^{4b}, R^{4c} and R^{4d} are given in Table 1

Table XVIII provides 782 compounds of formula Ir 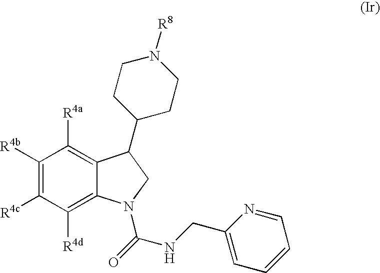
wherein the values of R⁸, R^{4a}, R^{4b}, R^{4c} and R^{4d} are given in Table 1

Table XIX provides 782 compounds of formula Is 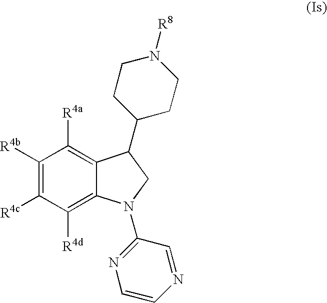
wherein the values of R⁸, R^{4a}, R^{4b}, R^{4c} and R^{4d} are given in Table 1

Table XX provides 782 compounds-of formula It 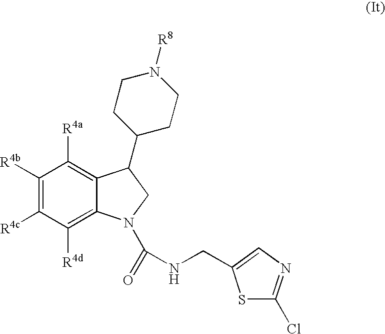
wherein the values of R⁸, R^{4a}, R^{4b}, R^{4c} and R^{4d} are given in Table 1

Table XXI provides 782 compounds of formula Iu 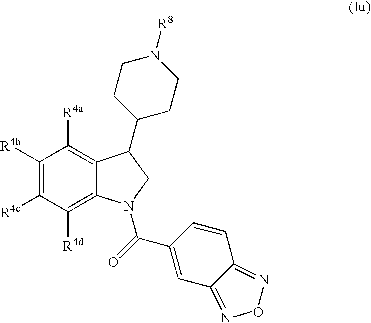
wherein the values of R⁸, R^{4a}, R^{4b}, R^{4c} and R^{4d} are given in Table 1

Table XXII provides 782 compounds of formula Iv 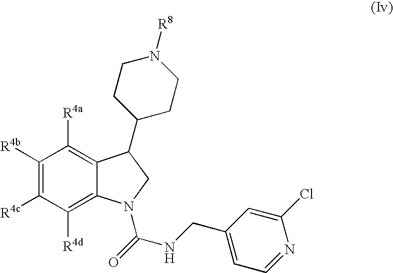
wherein the values of R⁸, R^{4a}, R^{4b}, R^{4c} and R^{4d} are given in Table 1

Table XXIII provides 782 compounds of formula Iw 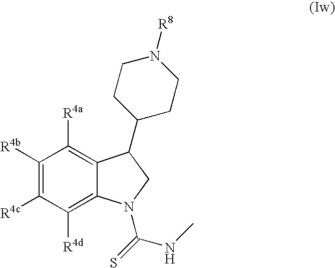
wherein the values of R⁸, R^{4a}, R^{4b}, R^{4c} and R^{4d} are given in Table 1

Table XXIV provides 782 compounds of formula Ix 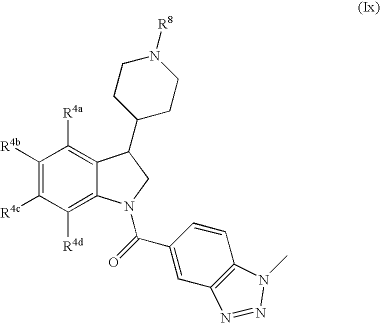
wherein the values of R⁸, R^{4a}, R^{4b}, R^{4c} and R^{4d} are given in Table 1

Table XXV provides 782 compounds of formula Iy 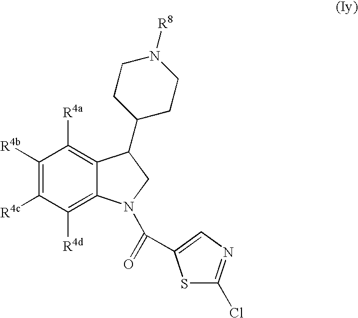
wherein the values of R⁸, R^{4a}, R^{4b}, R^{4c} and R^{4d} are given in Table 1

Table XXVII provides 782 compounds of formula Iaa 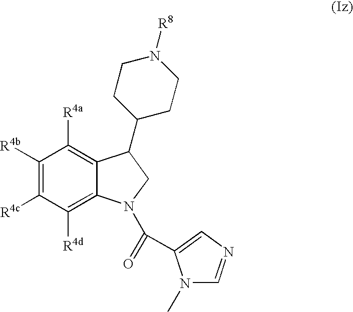
wherein the values, of R⁸, R^{4a}, R^{4b}, R^{4c} and R^{4d} are given in Table 1

Table XXVII provides 782 compounds of formula Iaa 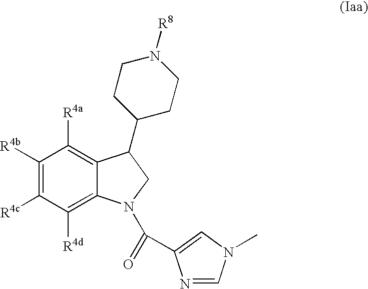
wherein the values of R⁸, R^{4a}, R^{4b}, R^{4c} and R^{4d} are given in Table 1

Table XXVIII provides 782 compounds of formula Iab 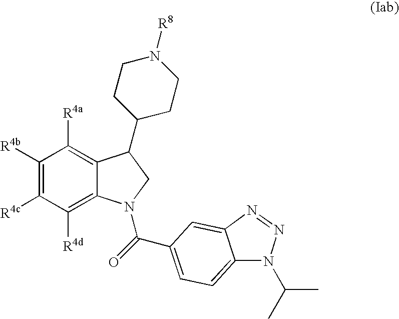
wherein the values of R⁸, R^{4a}, R^{4b}, R^{4c} and R^{4d} are given in Table 1

Table XXI provides 782 compounds of formula Iac 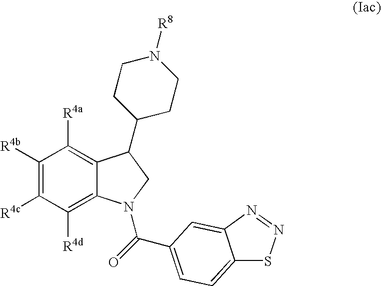
wherein the values of R⁸, R^{4a}, R^{4b}, R^{4c} and R^{4d} are given in Table 1

Table XXX provides 782 compounds of formula Iad 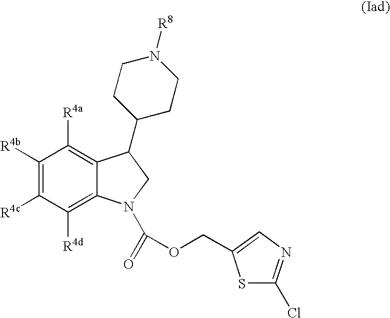
wherein the values of R⁸, R^{4a}, R^{4b}, R^{4c} and R^{4d} are given in Table 1

Table XXXI provides 782 compounds of formula Iae 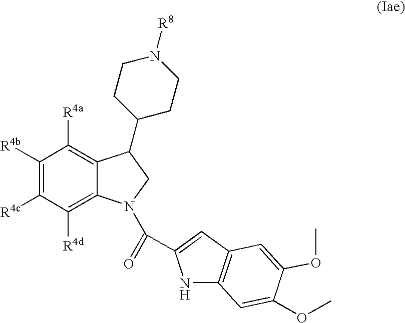
wherein the values of R⁸, R^{4a}, R^{4b}, R^{4c} and R^{4d} are given in Table 1

Table XXXII provides 782 compounds of formula Iaf 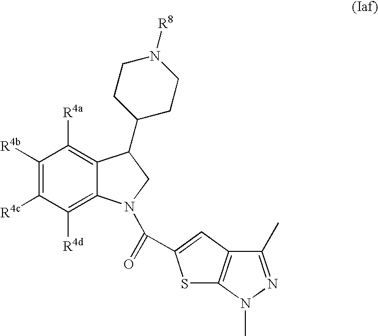
wherein the values of R⁸, R^{4a}, R^{4b}, R^{4c} and R^{4d} are given in Table 1

Table XXXIII provides 782 compounds of formula Iag 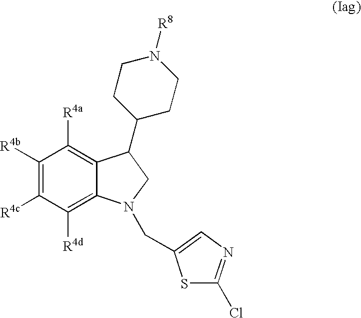
wherein the values of R⁸, R^{4a}, R^{4b}, R^{4c} and R^{4d} are given in Table 1

Table XXXIV provides 782 compounds of formula Iah 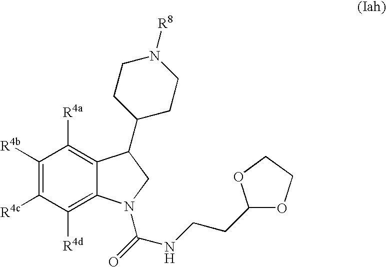
wherein the values of R⁸, R^{4a}, R^{4b}, R^{4c} and R^{4d} are given in Table 1

Table XXXV provides 782 compounds of formula Iai 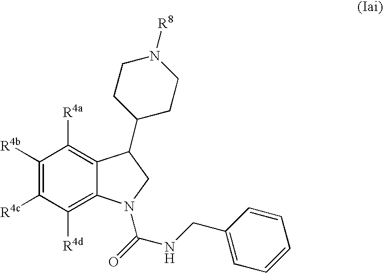
wherein the values of R⁸, R^{4a}, R^{4b}, R^{4c} and R^{4d} are given in Table 1

Table XXXVI provides 782 compounds of formula Iaj 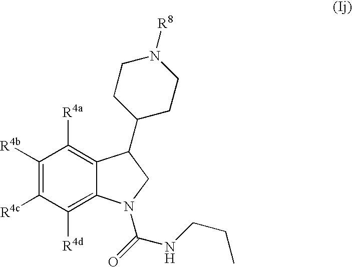
wherein the values of R⁸, R^{4a}, R^{4b}, R^{4c} and R^{4d} are given in Table 1

Table XXXVI provides 782 compounds of formula Iak 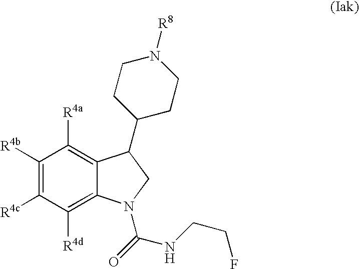
wherein the values of R⁸, R^{4a}, R^{4b}, R^{4c} and R^{4d} are given in Table 1

Table XXXVIII provides 782 compounds of formula Ial 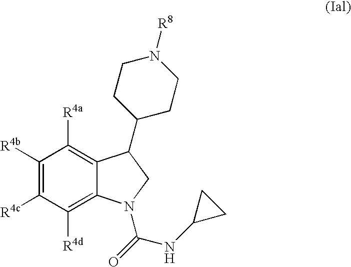
wherein the values of R⁸, R^{4a}, R^{4b}, R^{4c} and R^{4d} are given in Table 1

Table XXXIX provides 782 compounds of formula Iam 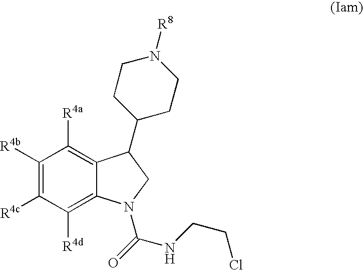
wherein the values of R⁸, R^{4a}, R^{4b}, R^{4c} and R^{4d} are given in Table 1

Table XL provides 782 compounds of formula Ian 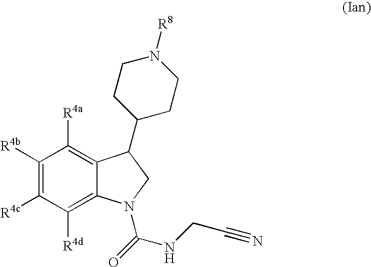
wherein the values of R⁸, R^{4a}, R^{4b}, R^{4c} and R^{4d} are given in Table 1
Table XLI provides 782 compounds of formula Iao 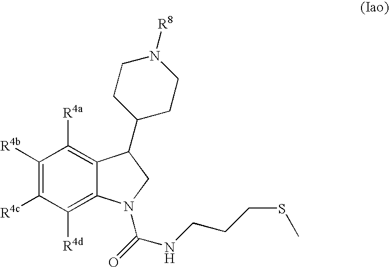
wherein the values of R⁸, R^{4a}, R^{4b}, R^{4c} and R^{4d} are given in Table 1

Table XLII provides 782 compounds of formula Iap 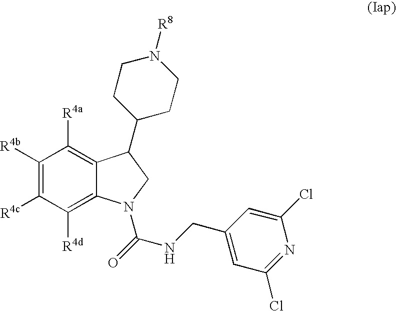
wherein the values of R⁸, R^{4a}, R^{4b}, R^{4c} and R^{4d} are given in Table 1

Table XLIII provides 782 compounds of formula Iaq 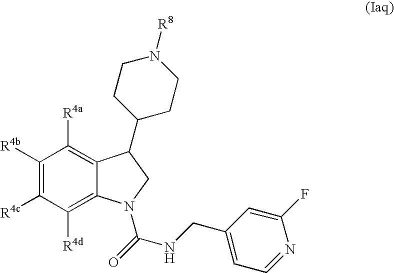
wherein the values of R⁸, R^{4a}, R^{4b}, R^{4c} and R^{4d} are given in Table 1.

Table XLIV provides 782 compounds of formula Iar 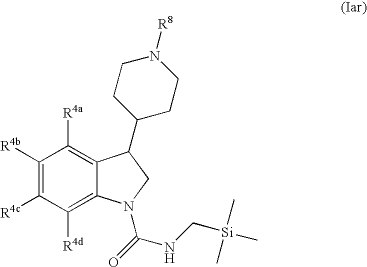
wherein the values of R⁸, R^{4a}, R^{4b}, R^{4c} and R^{4d} are given in Table 1.

Table XLV provides 782 compounds of formula Ias 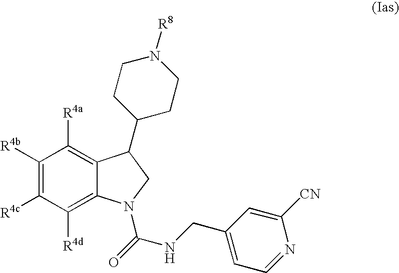
wherein the values of R⁸, R^{4a}, R^{4b}, R^{4c} and R^{4d} are given in Table 1.

Table XLVI provides 782 compounds of formula Iat 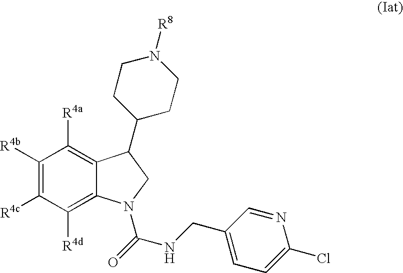
wherein the values of R⁸, R^{4a}, R^{4b}, R^{4c} and R^{4d} are given in Table 1.

Table XLVII provides 782 compounds of formula Iau 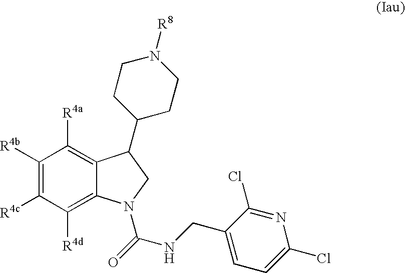
wherein the values of R⁸, R^{4a}, R^{4b}, R^{4c} and R^{4d} are given in Table 1.

Table XLVIII provides 782 compounds of formula Iav 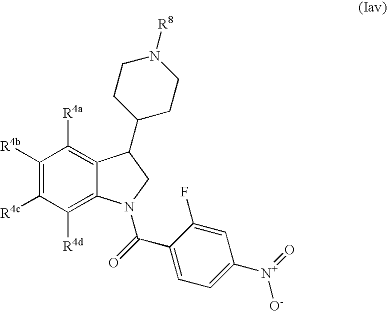
wherein the values of R⁸, R^{4a}, R^{4b}, R^{4c} and R^{4d} are given in Table 1.

Table XLIX provides 782 compounds of formula Iaw 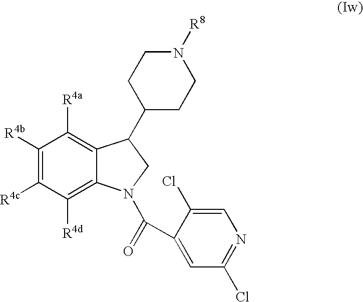
wherein the values of R⁸, R^{4a}, R^{4b}, R^{4c} and R^{4d} are given in Table 1.

Table L provides 782 compounds of formula Iax 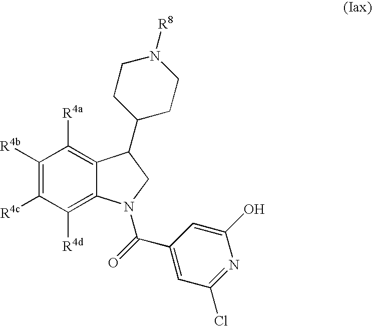
wherein the values of R⁸, R^{4a}, R^{4b}, R^{4c} and R^{4d} are given in Table 1.

Table LI provides 782 compounds of formula Iay 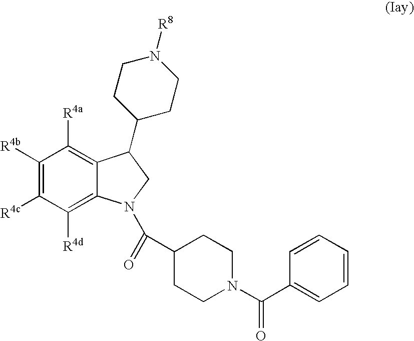
wherein the values of R⁸, R^{4a}, R^{4b}, R^{4c} and R^{4d} are given in Table 1.

Table LII provides 782 compounds of formula Iaz 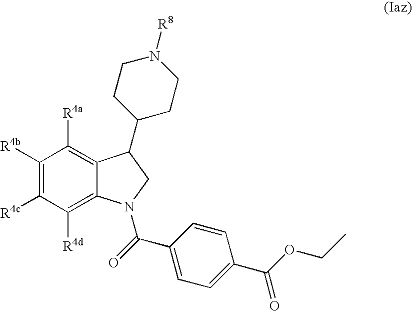
wherein the values of R⁸, R^{4a}, R^{4b}, R^{4c} and R^{4d} are given in Table 1.

Table LIII provides 782 compounds of formula Iba 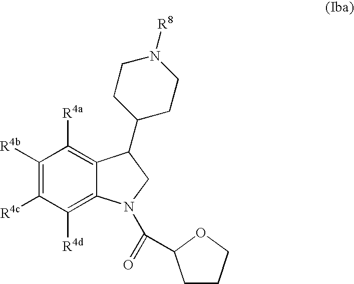
wherein the values of R⁸, R^{4a}, R^{4b}, R^{4c} and R^{4d} are given in Table 1.

Table LIV provides 782 compounds of formula Ibb 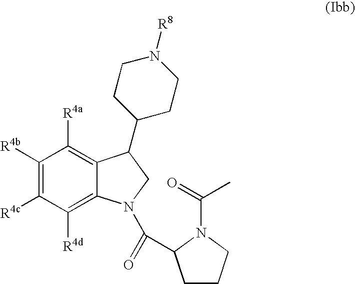
wherein the values of R⁸, R^{4a}, R^{4b}, R^{4c} and R^{4d} are given in Table 1.

Table LV provides 782 compounds of formula Ibc 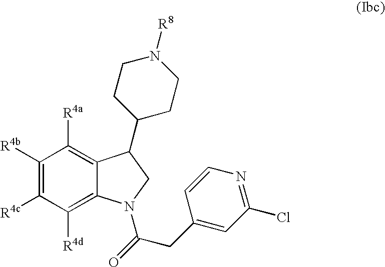
wherein the values of R⁸, R^{4a}, R^{4b}, R^{4c} and R^{4d} are given in Table 1.

Table LVI provides 782 compounds of formula Ibd 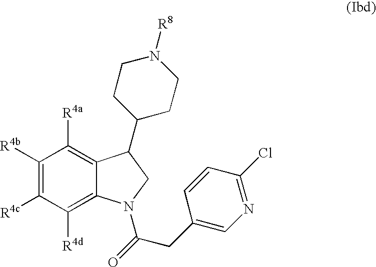
wherein the values of R⁸, R^{4a}, R^{4b}, R^{4c} and R^{4d} are given in Table 1.

Table LVII provides 782 compounds of formula Ibe 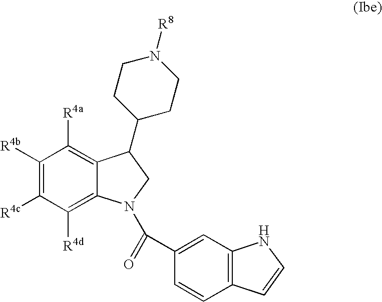
wherein the values of R⁸, R^{4a}, R^{4b}, R^{4c} and R^{4d} are given in Table 1.

Table LVIII provides 782 compounds of formula Ibf 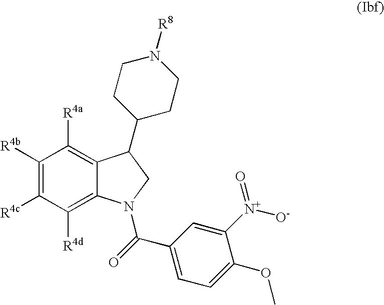
wherein the values of R⁸, R^{4a}, R^{4b}, R^{4c} and R^{4d} are given in Table 1.

Table LIX provides 782 compounds of formula Ibg 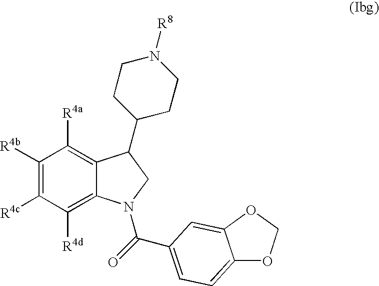
wherein the values of R⁸, R^{4a}, R^{4b}, R^{4c} and R^{4d} are given in Table 1.

Table LX provides 782 compounds of formula Ibh 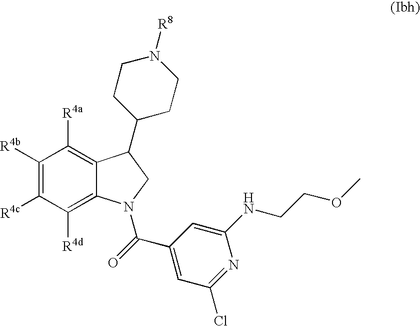
wherein the values of R⁸, R^{4a}, R^{4b}, R^{4c} and R^{4d} are given in Table 1.

Table LXI provides 782 compounds of formula Ibi 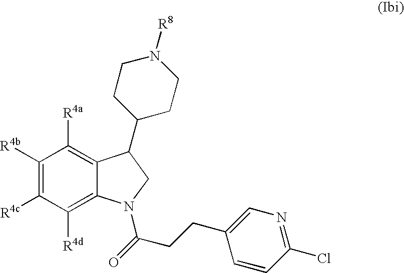
wherein the values of R⁸, R^{4a}, R^{4b}, R^{4c} and R^{4d} are given in Table 1.

Table LXII provides 782 compounds of formula Ibj 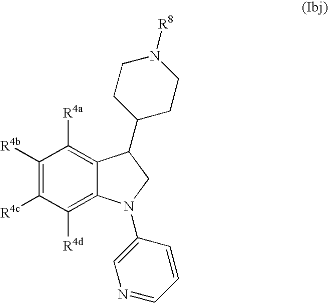
wherein the values of R⁸, R^{4a}, R^{4b}, R^{4c} and R^{4d} are given in Table 1.

Table LXIII provides 782 compounds of formula Ibk 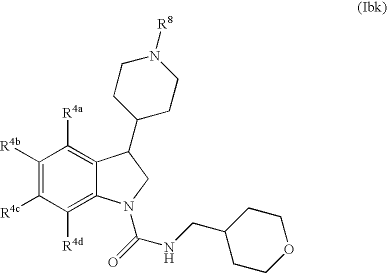
wherein the values of R⁸, R^{4a}, R^{4b}, R^{4c} and R^{4d} are given in Table 1.

Table LXIV provides 782 compounds of formula Ibl 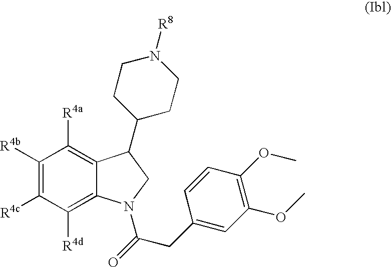
wherein the values of R⁸, R^{4a}, R^{4b}, R^{4c} and R^{4d} are given in Table 1.

Table LXV provides 782 compounds of formula Ibm 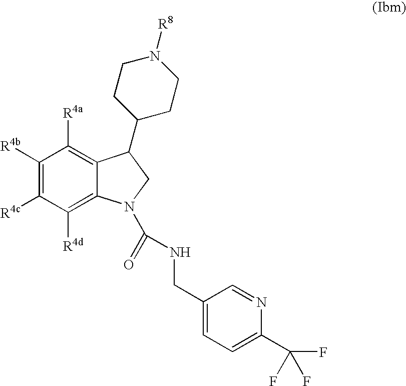
wherein the values of R⁸, R^{4a}, R^{4b}, R^{4c} and R^{4d} are given in Table 1.

Table LXVI provides 782 compounds of formula Ibn 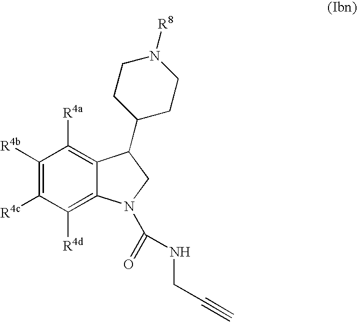
wherein the values of R⁵, R^{4a}, R^{4b}, R^{4c} and R^{4d} are given in Table 1.

Table LXVII provides 782 compounds of formula Ibo 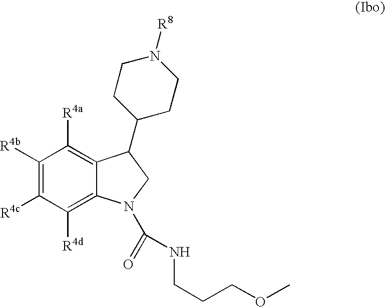
wherein the values of R⁸, R^{4a}, R^{4b}, R^{4c} and R^{4d} are given in Table 1.

Table LXVMI provides 782 compounds of formula Ibp 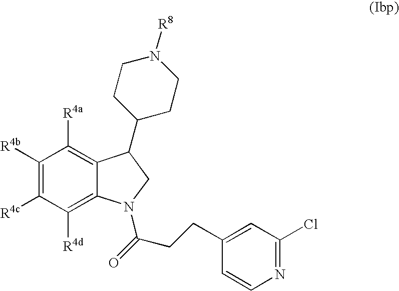
wherein the values of R⁸, R^{4a}, R^{4b}, R^{4c} and R^{4d} are given in Table 1.

Table LXIX provides 782 compounds of formula Ica 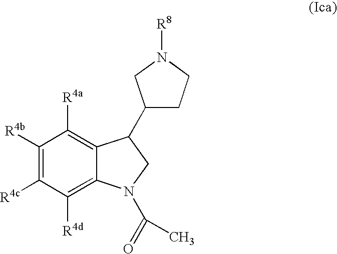
wherein the values of R⁸, R^{4a}, R^{4b}, R^{4c} and R^{4d} are given in Table 1.

Table LXX provides 782 compounds of formula Icb 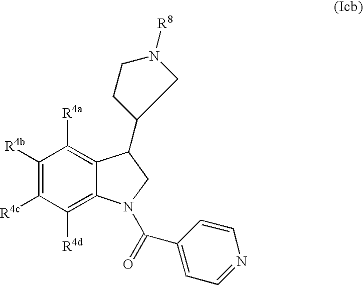
wherein the values of R⁸, R^{4a}, R^{4b}, R^{4c} and R^{4d} are given in Table 1.

Table LXXI provides 782 compounds of formula Icc 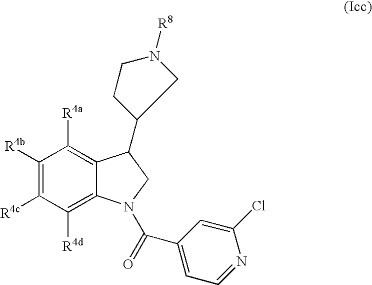
wherein the values of R⁸, R^{4a}, R^{4b}, R^{4c} and R^{4d} are given in Table 1.

Table LXXII provides 782 compounds of formula Icd 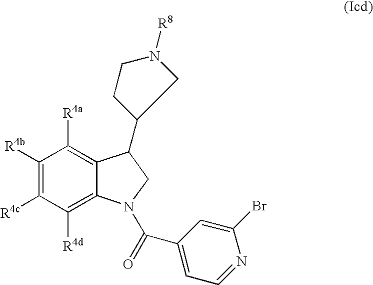
wherein the values of R⁸, R^{4a}, R^{4b}, R^{4c} and R^{4d} are given in Table 1.

Table LXXII provides 782 compounds of formula Ice 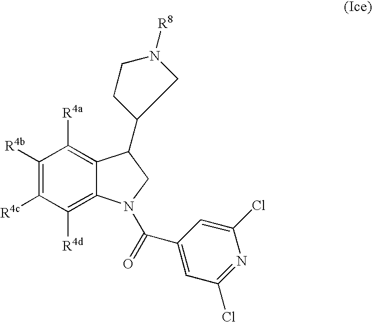
wherein the values of R⁸, R^{4a}, R^{4b}, R^{4c} and R^{4d} are given in Table 1.

Table LXXIV provides 782 compounds of formula Icf 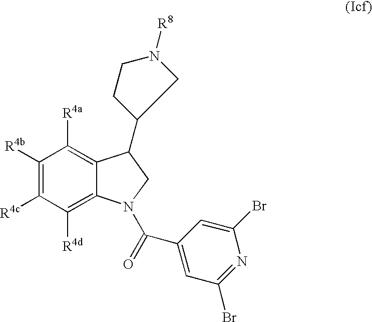
wherein the values of R⁸, R^{4b}, R^{4c} and R^{4d} are given in Table 1.

Table LXXV provides 782 compounds of formula Icg 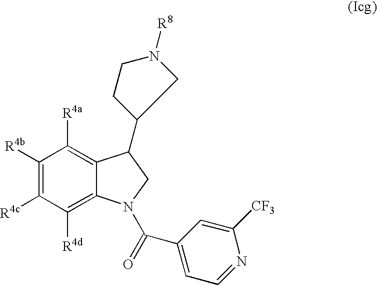
wherein the values of R⁸, R^{4a}, R^{4b}, R^{4c} and R^{4d} are given in Table 1.

Table LXXVI provides 782 compounds of formula Ich 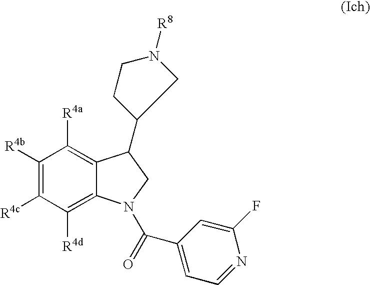
wherein the values of R⁸, R^{4a}, R^{4b}, R^{4c} and R^{4d} are given in Table 1.

Table LXXVII provides 782 compounds of formula Ici 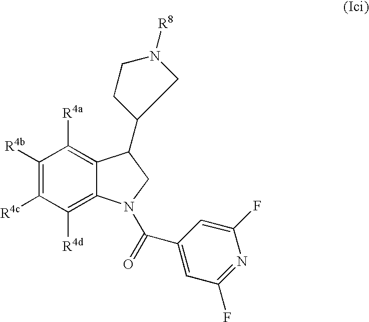
wherein the values of R⁸, R^{4a}, R^{4b}, R^{4c} and R^{4d} are given in Table 1.

Table LXXVIII provides 782 compounds of formula Icj 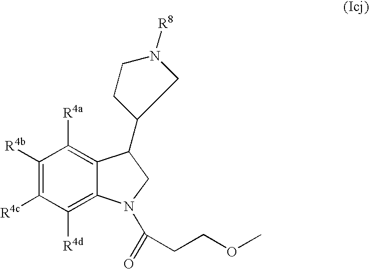
wherein the values of R⁸, R^{4a}, R^{4b}, R^{4c} and R^{4d} are given in Table 1.

Table LXXXIX provides 782 compounds of formula Ick 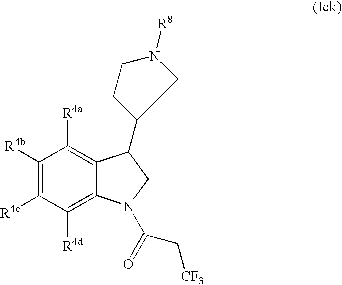
wherein the values of R⁸, R^{4a}, R^{4b}, R^{4c} and R^{4d} are given in Table 1/

Table LXXX provides 782 compounds of formula Icl 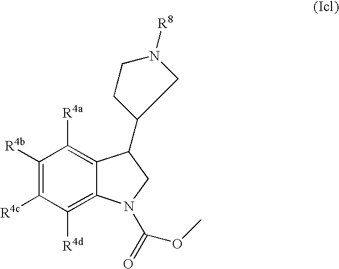
wherein the values of R⁸, R^{4a}, R^{4b}, R^{4c} and R^{4d} are given in Table 1.

Table LXXXI provides 782 compounds of formula Icm 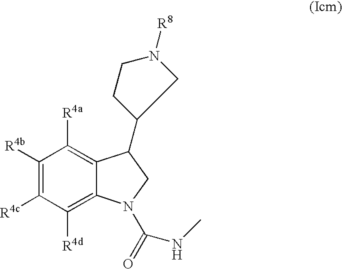
wherein the values of R⁸, R^{4a}, R^{4b}, R^{4c} and R^{4d} are given in Table 1.

Table LXXXII provides 782 compounds of formula Icn 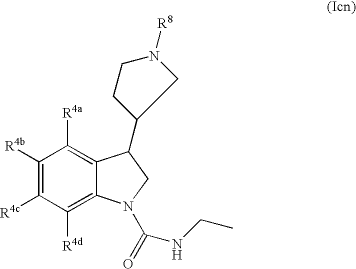
wherein the values of R⁸, R^{4a}, R^{4b}, R^{4c} and R^{4e} are given in Table 1.

Table LXXXIII provides 782 compounds of formula Ico 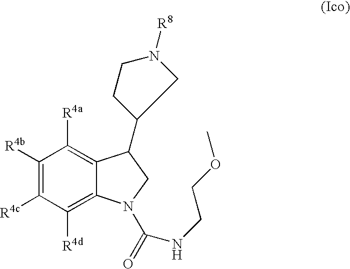
wherein the values of R⁸, R^{4a}, R^{4b}, R^{4c} and R^{4d} are given in Table 1.

Table LXXXIV provides 782 compounds of formula Icp 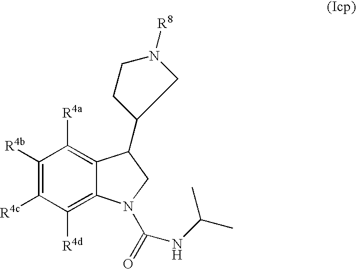
wherein the values of R⁸, R^{4a}, R^{4b}, R^{4c} and R^{4d} are given in Table 1.

Table LXXXV provides 782 compounds of formula Icq 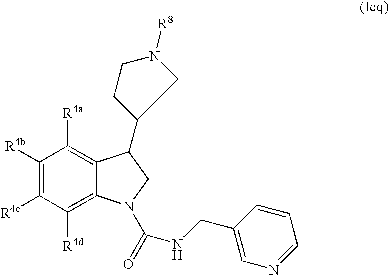
wherein the values of R⁸, R^{4a}, R^{4b}, R^{4c} and R^{4d} are given in Table 1.

Table LXXXVI provides 782 compounds of formula Icr 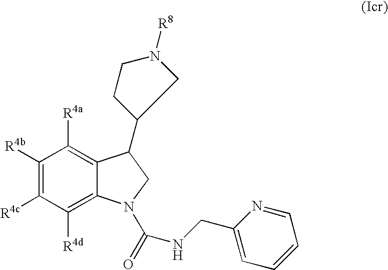
wherein the values of R⁸, R^{4a}, R^{4b}, R^{4c} and R^{4d} are given in Table 1.

Table LXXXVII provides 782 compounds of formula Ics 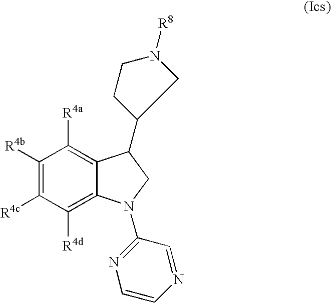
wherein the values of R⁸, R^{4a}, R^{4b}, R^{4c} and R^{4d} are given in Table 1.

Table LXXXVIII provides 782 compounds of formula Ict 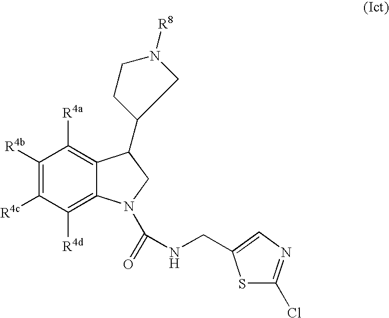
wherein the values of R⁸, R^{4a}, R^{4b}, R^{4c} and R^{4d} are given in Table 1.

Table LXXXIX provides 782 compounds of formula Icu 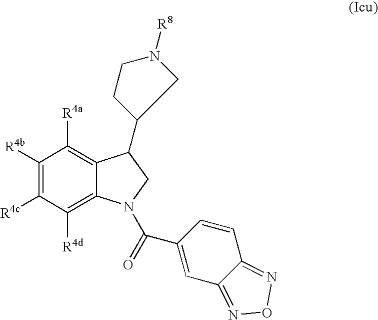
wherein the values of R⁸, R^{4a}, R^{4b}, R^{4c} and R^{4d} are given in Table 1.

Table XC provides 782 compounds of formula Icv 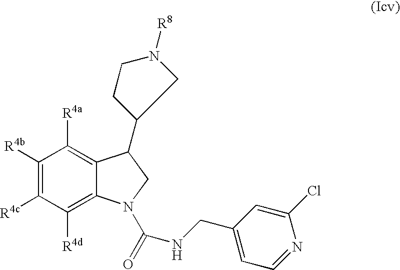
wherein the values of R⁸, R^{4a}, R^{4b}, R^{4c} and R^{4d} are given in Table 1.

Table XCI provides 782 compounds of formula Icw 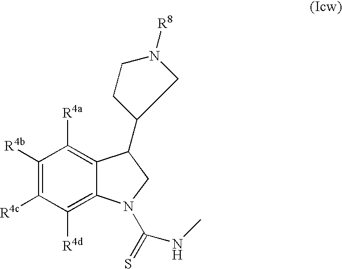
wherein the values of R⁸, R^{4a}, R^{4b}, R^{4c} and R^{4d} are given in Table 1.

Table XCII provides 782 compounds of formula Icx 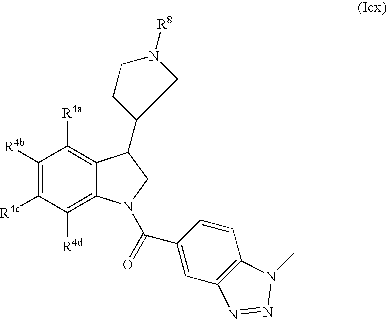
wherein the values of R⁸, R^{4a}, R^{4b}, R^{4c} and R^{4d} are given in Table 1.

Table XCIII provides 782 compounds of formula Icy 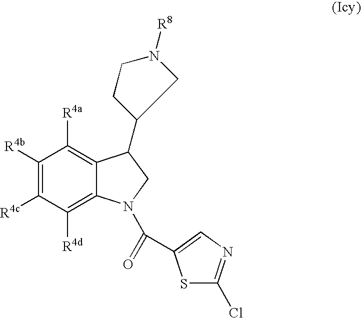
wherein the values of R⁸, R^{4a}, R^{4b}, R^{4c} and R^{4d} are given in Table 1.

Table XCIV provides 782 compounds of formula Icz 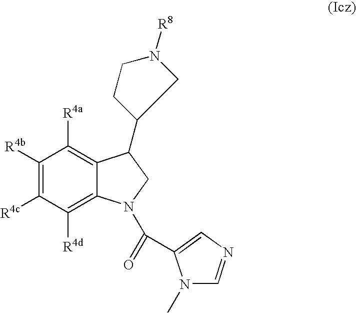
wherein the values of R⁸, R^{4a}, R^{4b}, R^{4c} and R^{4d} are given in Table 1.

Table XCV provides 782 compounds of formula Ida 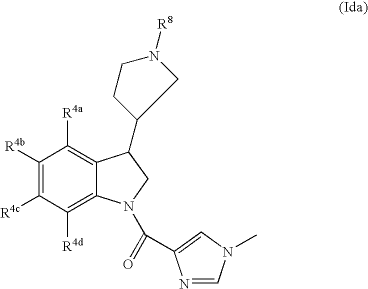
wherein the values of R⁸, R^{4a}, R^{4b}, R^{4c} and R^{4d} are given in Table 1.

Table XCVI provides 782 compounds of formula Idb 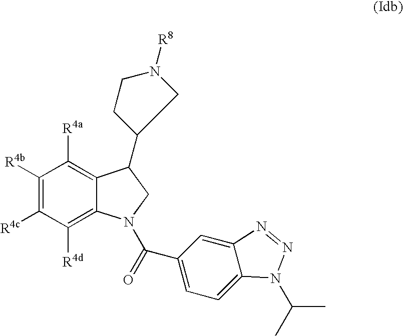
wherein the values of R⁸, R^{4a}, R^{4b}, R^{4c} and R^{4d} are given in Table 1.

Table XCVII provides 782 compounds of formula Idc 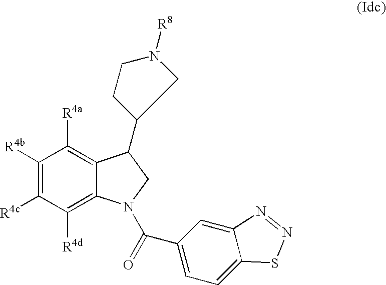
wherein the values of R⁸, R^{4a}, R^{4b}, R^{4c} and R^{4d} are given in Table 1.

Table XCVIII provides 782 compounds of formula Idd 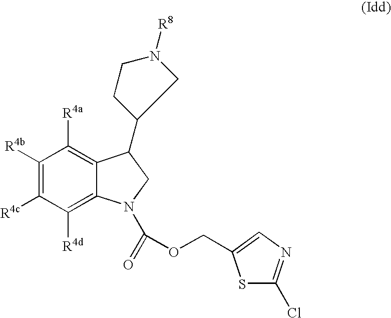
wherein the values of R⁸, R^{4a}, R^{4b}, R^{4c} and Re are given in Table 1.

Table XCIX provides 782 compounds of formula Ide 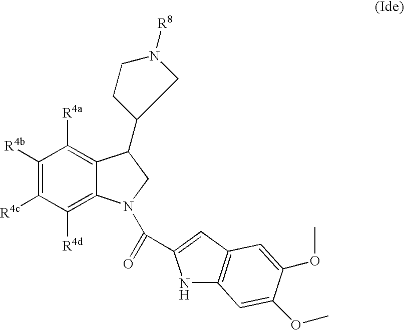
wherein the values of R⁸, R^{4a}, R^{4b}, R^{4c} and R^{4d} are given in Table 1.

Table C provides 782 compounds of formula Idf 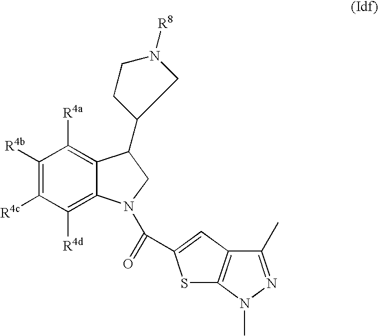
wherein the values of R⁸, R^{4a}, R^{4b}, R^{4c} and R^{4d} are given in Table 1.

Table CI provides 782 compounds of formula Idg 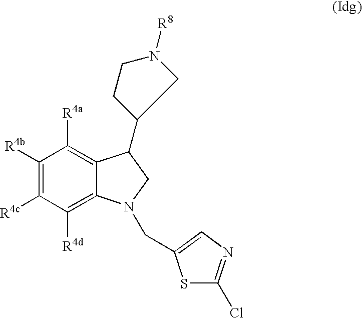
wherein the values of R⁸, R^{4a}, R^{4b}, R^{4c} and R^{4d} are- given in Table 1.

Table CII provides 782 compounds of formula Idh 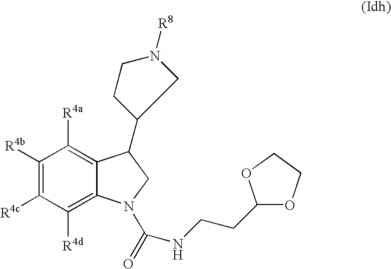
wherein the values of R⁸, R^{4a}, R^{4b}, R^{4c} and R^{4d} are given in Table 1.

Table CIII provides 782 compounds of formula Idi 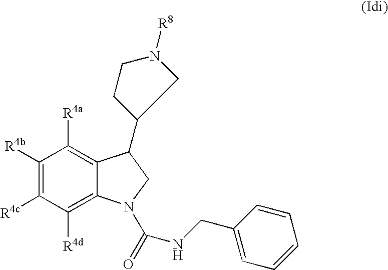
wherein the values of R⁸, R^{4a}, R^{4b}, R^{4c} and R^{4d} are given in Table 1.

Table CIV provides 182 compounds of formula Idj 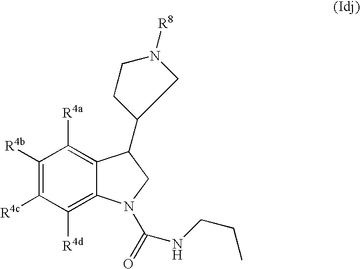
wherein the values of R⁸, R^{4a}, R^{4b}, R^{4c} and R^{4d} are given in Table 1.

Table CV provides 782 compounds of formula Idk 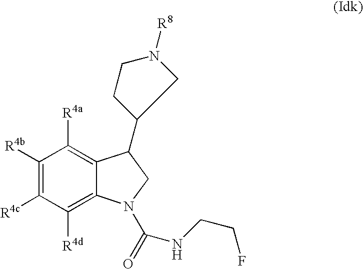
wherein the values of R⁸, R^{4a}, R^{4b}, R^{4c} and R^{4d} are given in Table 1.

Table CVI provides 782 compounds of formula Idl 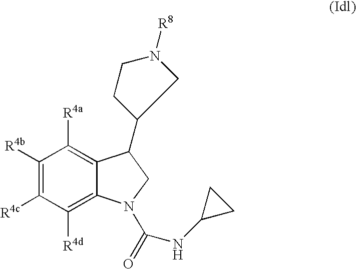
wherein the values of R⁸, R^{4a}, R^{4b}, R^{4c} and R^{4d} are given in Table 1.

Table CVII provides 782 compounds of formula Idm 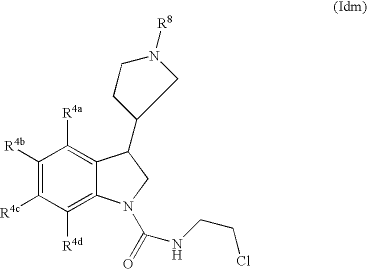
wherein the values of R⁸, R^{4a}, R^{4b}, R^{4c} and R^{4d} are given Table 1.

Table CVIII provides 782 compounds of formula Idn 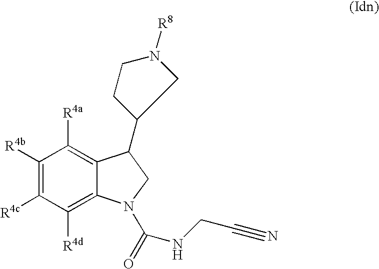
wherein the values of R⁸, R^{4a}, R^{4b}, R^{4c} and R^{4d} are given in Table 1.

Table CIX provides 782 compounds of formula Ido 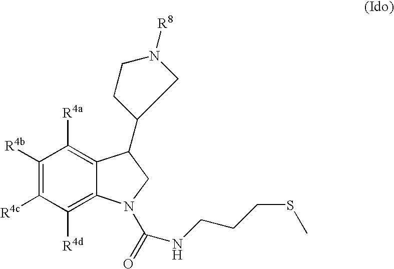
wherein the values of R⁸, R^{4a}, R^{4b}, R^{4c} and R^{4d} are given in Table 1.

Table CXI provides 782 compounds of formula Idp 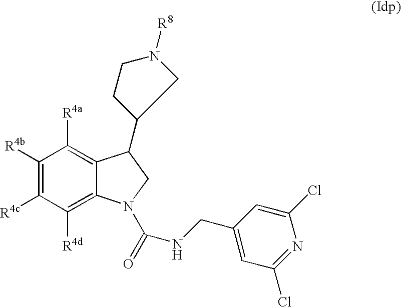
wherein the values of R⁸, R^{4a}, R^{4b}, R^{4c} and R^{4d} are given in Table 1.

Table CXI provides 782 compounds of formula Idq 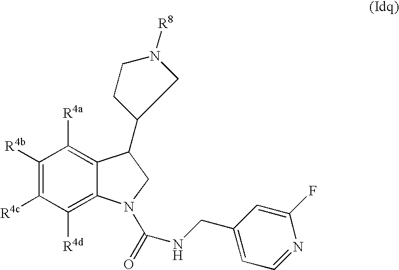
wherein the values of R⁸, R^{4a}, R^{4b}, R^{4c} and R^{4d} are given in Table 1.

Table CXII provides 782 compounds of formula Idr 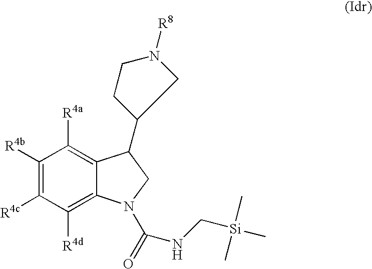
wherein the values of R⁸, R^{4a}, R^{4b}, R^{4c} and R^{4d} are given in Table 1.

Table CXIII provides 782 compounds of formula Ids 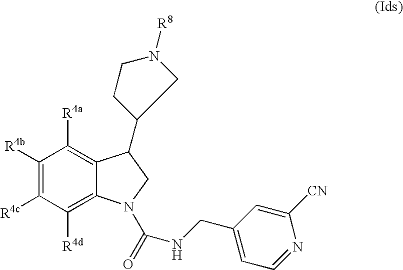
wherein the values of R⁸, R^{4a}, R^{4b}, R^{4c} and R^{4d} are given in Table 1.

Table CXIV provides 782 compounds of formula Idt 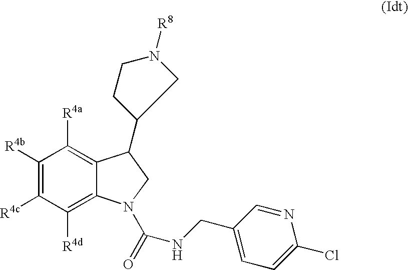
wherein the values of R⁸, R^{4a}, R^{4b}, R^{4c} and R^{4d} are given in Table 1.

Table CXV provides 782 compounds of formula Idu 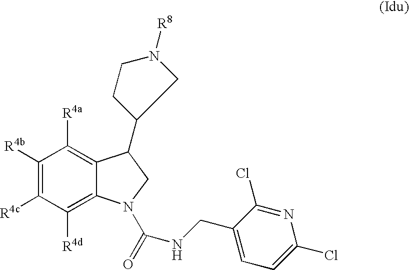
wherein the values of R⁸, R^{4a}, R^{4b}, R^{4c} and R^{4d} are given in Table 1.

Table CXVI provides 782 compounds of formula Idv 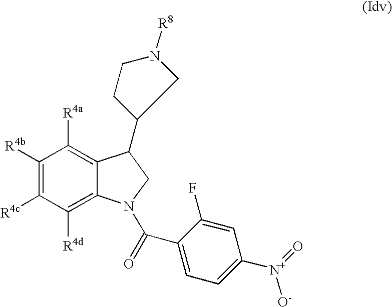
wherein the values of R⁸, R^{4a}, R^{4b}, R^{4c} and R^{4d} are given in Table 1.

Table CXVII provides 782 compounds of formula Idw 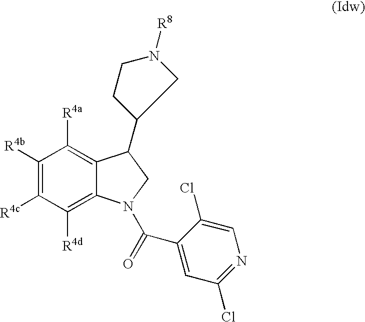
wherein the values of R⁸, R^{4a}, R^{4b}, R^{4c} and R^{4d} are given in Table 1.

Table CXVIII provides 782 compounds of formula Idx 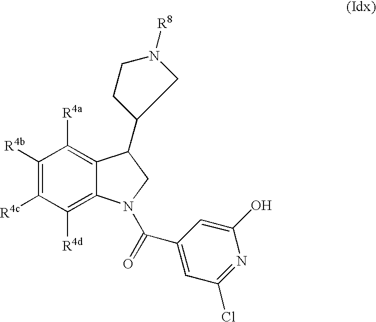
wherein the values of R⁸, R^{4a}, R^{4b}, R^{4c} and R^{4d} are given in Table 1.

Table CXIX provides 782 compounds of formula Idy 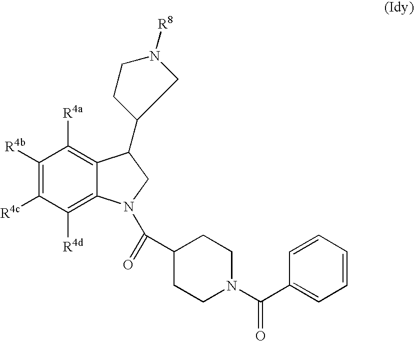
wherein the values of R⁸, R^{4a}, R^{4b}, R^{4c} and R^{4d} are given in Table 1.

Table CXX provides 782 compounds of formula Idz 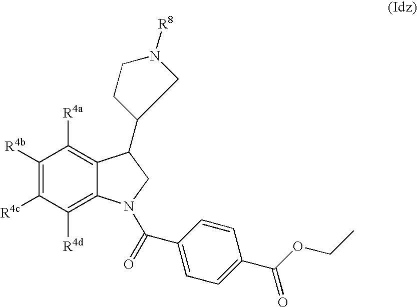
wherein the values of R⁸, R^{4a}, R^{4b}, R^{4c} and R^{4d} are given in Table 1.

Table CXXI provides 782 compounds of formula Iea 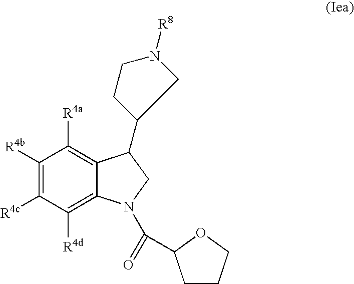
wherein the values of R⁸, R^{4a}, R^{4b}, R^{4c}, and R^{4d} are given in Table 1.

Table CXXII provides 782 compounds of formula Ieb 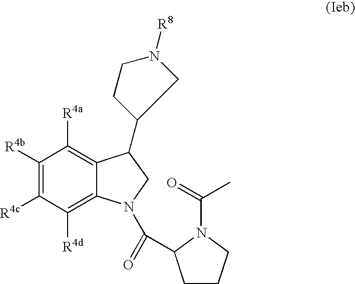
wherein the values of R⁸, R^{4a}, R^{4b}, R^{4c} and R^{4d} are given in Table 1.

Table CXXIII provides 782 compounds of formula Iec 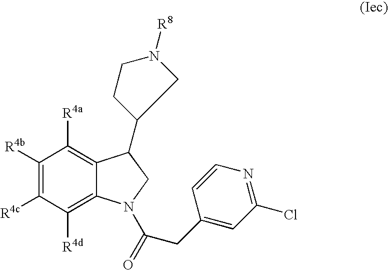
wherein the values of R⁸, R^{4a}, R^{4b}, R^{4c} and R^{4d} are given in Table 1.

Table CXXIV provides 782 compounds of formula Ied 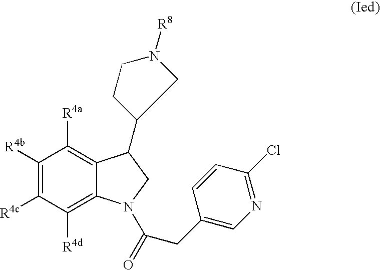
wherein the values of R⁸, R^{4a}, R^{4b}, R^{4c} and R^{4d} are given in Table 1.

Table CXXV provides 782 compounds of formula Iee 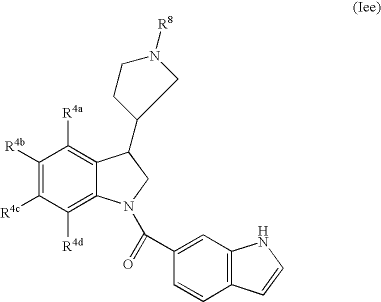
wherein the values of R⁸, R^{4a}, R^{4b}, R^{4c} and R^{4d} are given in Table 1.

Table CXXVI provides 782 compounds of formula Ief 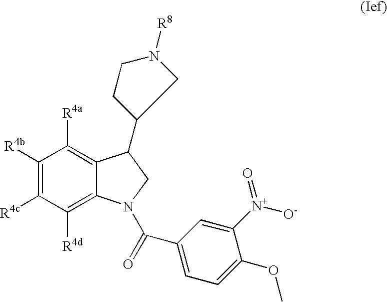
wherein the values of R⁸, R^{4a}, R^{4b}, R^{4c} and R^{4d} are given in Table 1.

Table CXXVII provides 782 compounds of formula Ieg 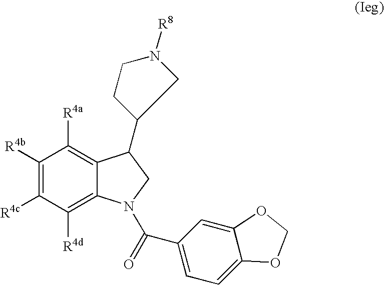
wherein the values of R⁸, R^{4a}, R^{4b}, R^{4c} and R^{4d} are given in Table 1.

Table CXXIII provides 782 compounds of formula Ieh 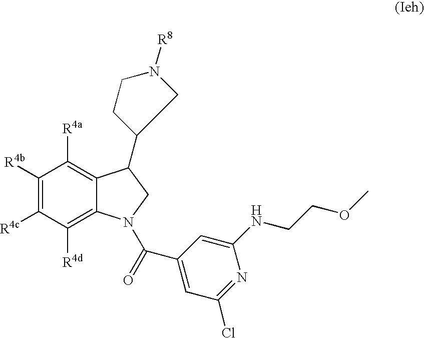
wherein the values of R⁸, R^{4a}, R^{4b}, R^{4c} and R^{4d} are given in Table 1.

Table CXXIX provides 782 compounds of formula Iei 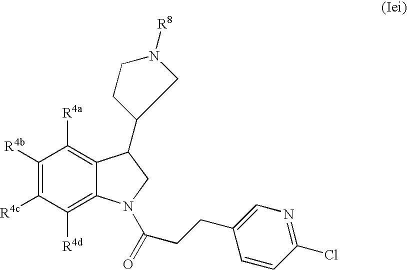
wherein the values of R⁸, R^{4a}, R^{4b}, R^{4c} and R^{4d} are given in Table 1.

Table CXXX provides 782 compounds of formula Iej 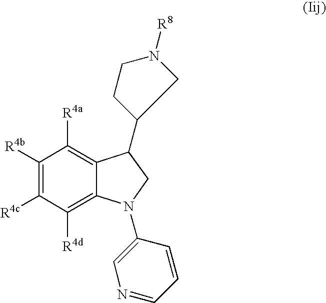
wherein the values of R⁸, R^{4a}, R^{4b}, R^{4c} and R^{4d} are given in Table 1.

Table CXXXI provides 782 compounds of formula Iek 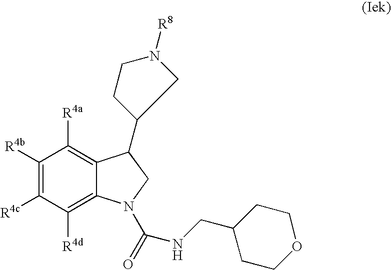
wherein the values of R⁸, R^{4a}, R^{4b}, R^{4c} and R^{4d} are given in Table 1.

Table CVXXII provides 782 compounds of formula Iel 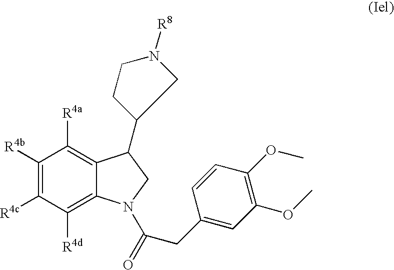
wherein the values of R⁸, R^{4a}, R^{4b}, R^{4c} and R^{4d} are given in Table 1.

Table CXXXIII provides 782 compounds of formula Iem 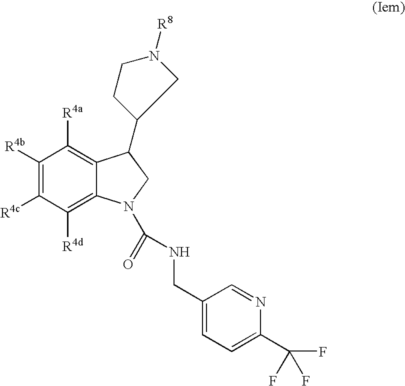
wherein the values of R⁸, R^{4a}, R^{4b}, R^{4c} and R^{4d} are given in Table 1.

Table CXXXIV provides 782 compounds of formula Ien 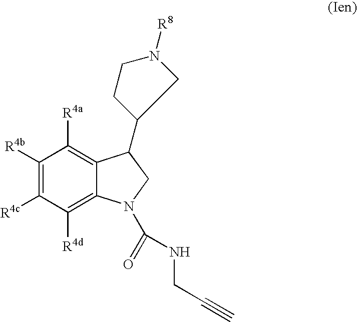
wherein the values of R⁸, R^{4a}, R^{4b}, R^{4c} and R^{4d} are given in Table 1.

Table CXXXV provides 782 compounds of formula Ieo 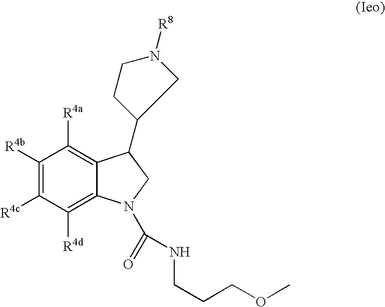
wherein the values of R⁸, R^{4a}, R^{4b}, R^{4c} and R^{4d} are given in Table 1.

Table CCI provides 782, compounds of formula Ifa 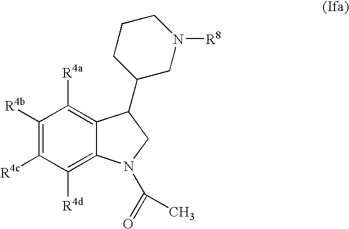
wherein the values of R⁸, R^{4a}, R^{4b}, R^{4c} and R^{4d} are given in Table 1

Table CCII provides 782 compounds of formula Ifb 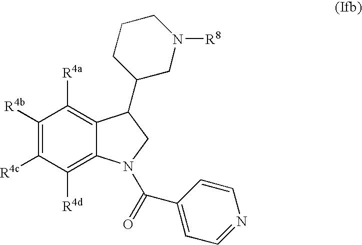
wherein the values of R⁸, R^{4a}, R^{4b}, R^{4c} and R^{4d} are given in Table 1

Table CCIII provides 782 compounds of formula Ifc 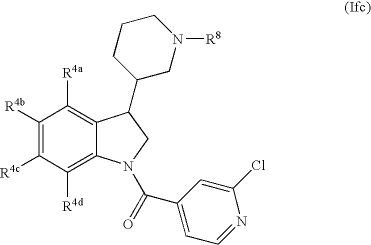
wherein the values of R⁸, R^{4a}, R^{4b}, R^{4c} and R^{4d} are given in Table 1

Table CCIV provides 782 compounds of formula Ifd 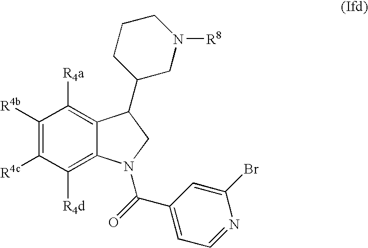
wherein the values of R⁸, R^{4a}, R^{4b}, R^{4c} and R^{4d} are given in Table 1

Table CCV provides 782 compounds of formula Ife 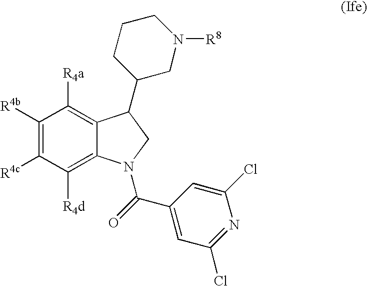
wherein the values of R⁸, R^{4a}, R^{4b}, R^{4c} and R^{4d} are given in Table 1

Table CCVI provides 782 compounds of formula Iff 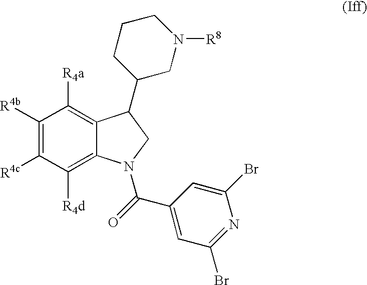
wherein the values of R⁸, R^{4a}, R^{4b}, R^{4c} and R^{4d} are given in Table 1

Table CCVII provides 782 compounds of formula Ifg 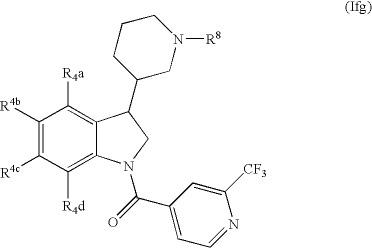
wherein the values of R⁸, R^{4a}, R^{4b}, R^{4c} and R^{4d} are given in Table 1

Table CCVIII provides 782 compounds of formula Ifh 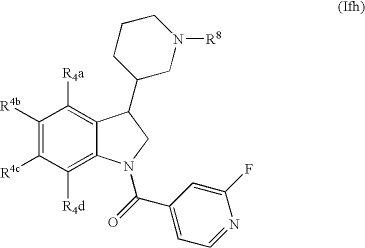
wherein the values of R⁸, R^{4a}, R^{4b}, R^{4c} and R^{4d} are given in Table 1

Table CCIX provides 782 compounds of formula Ifi 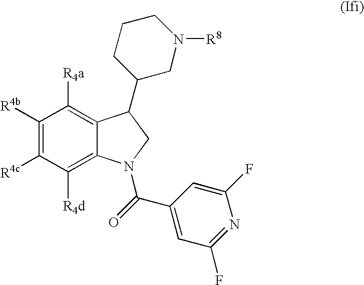
wherein the values of R⁸, R^{4a}, R^{4b}, R^{4c} and R^{4d} are given in Table 1

Table CCX provides 782 compounds of formula Ifj 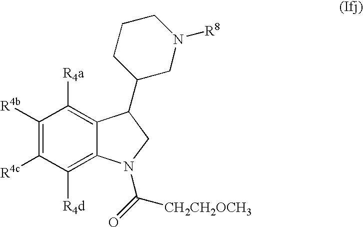
wherein the values of R⁸, R^{4a}, R^{4b}, R^{4c} and R^{4d} are given in Table 1

Table CCXI provides 782 compounds of formula Ifk 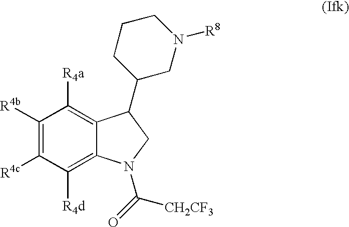
wherein the values of R⁸, R^{4a}, R^{4b}, R^{4c} and R^{4d} are given in Table 1

Table CCXII provides 782 compounds of formula Ifl 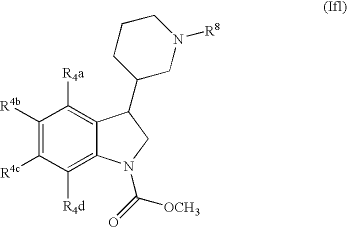
wherein the values of R⁸, R^{4a}, R^{4b}, R^{4c} and R^{4d} are given in Table 1

Table CCXIII provides 782 compounds of formula Ifm 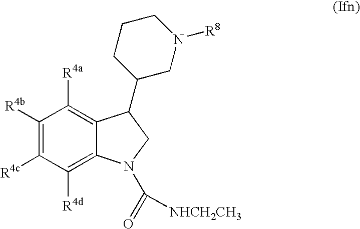
wherein the values of R⁸, R^{4a}, R^{4b}, R^{4c} and R^{4d} are given in Table 1

Table CCXIV provides 782 compounds of formula Ifn 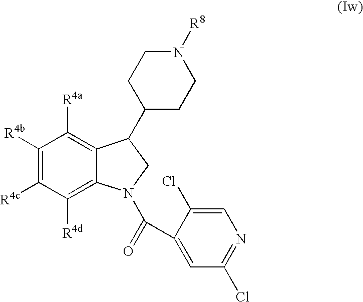
wherein the values of R⁸, R^{4a}, R^{4b}, R^{4c} and R^{4d} are given in Table 1

Table CCXV provides 782 compounds of formula Ifo 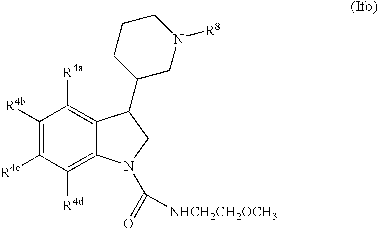
wherein the values of R⁸, R^{4a}, R^{4b}, R^{4c} and R^{4d} are given in Table 1

Table CCXVI provides 782 compounds of formula Ifp 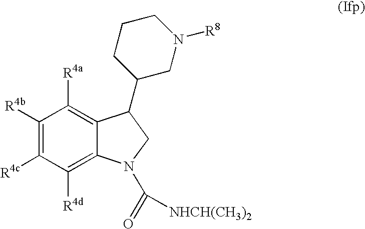
wherein the values of R⁸, R^{4a}, R^{4b}, R^{4c} and R^{4d} are given in Table 1

Table CCXVII provides 782 compounds of formula Ifq 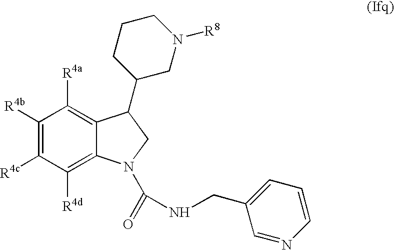
wherein the values of R⁸, R^{4a}, R^{4b}, R^{4c} and R^{4d} are given in Table 1

Table CCXVIII provides 782 compounds of formula Ifr 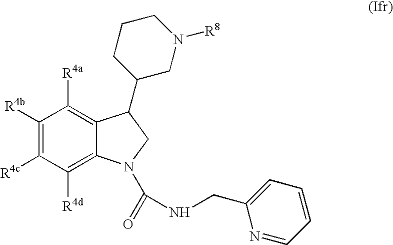
wherein the values of R⁸, R^{4a}, R^{4b}, R^{4c} and R^{4d} are given in Table 1

Table CCXIX provides 782 compounds of formula Ifs 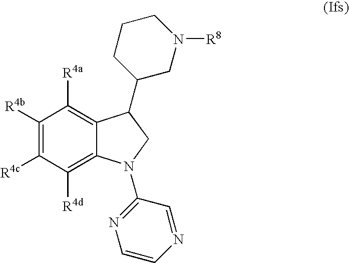
wherein the values of R⁸, R^{4a}, R^{4b}, R4c, and R^{4d} are given in Table 1

Table CCXX provides 782 compounds of formula Ift 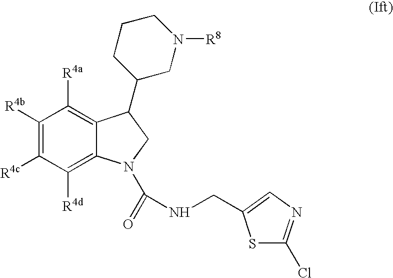
wherein the values of R⁸, R^{4a}, R^{4b}, R^{4c} and R^{4d} are given in Table 1

Table CCXXI provides 782 compounds of formula Ifu 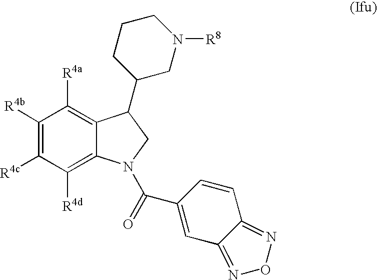
wherein the values of R⁸, R^{4a}, R^{4b}, R^{4c} and R^{4d} are given in Table 1

Table CCXXII provides 782 compounds of formula Ifv 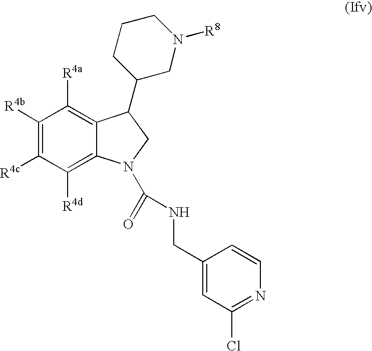
wherein the values of R⁸, R^{4a}, R^{4b}, R^{4c} and R^{4d} are given in Table 1

Table CCXXIII provides 782 compounds of formula Ifw 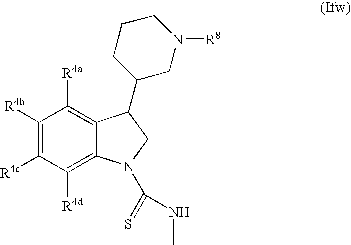
wherein the values of R⁸, R^{4a}, R^{4b}, R^{4c} and R^{4d} are given in Table 1

Table CCXXIV provides 782 compounds of formula Ifx 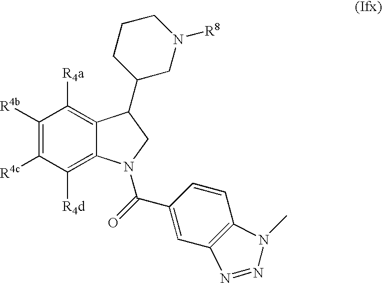
wherein the values of R⁸, R^{4a}, R^{4b}, R^{4c} and R^{4d} are given in Table 1

Table CCXXV provides 782 compounds of formula Ify 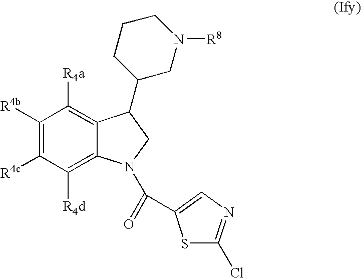
wherein the values of R⁸, R^{4a}, R^{4b}, R^{4c} and R^{4d} are given in Table 1

Table CCXXVI provides 782 compounds of formula Ifa 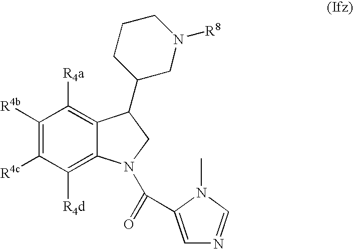
wherein the values of R⁸, R^{4a}, R^{4b}, R^{4c} and R^{4d} are given in Table 1

Table CCXXVII provides 782 compounds of formula Iga 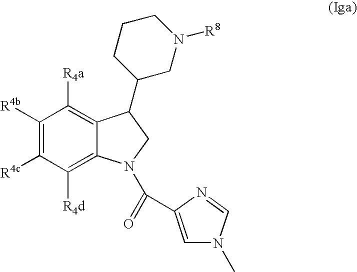
wherein the values of R⁸, R^{4a}, R^{4b}, R^{4c} and R^{4d} are given in Table 1

Table CCXXVIII provides 782 compounds of formula Igb 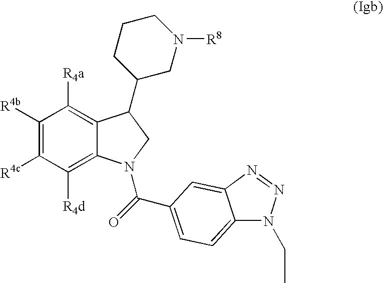
wherein the values of R⁸, R^{4a}, R^{4b}, R^{4c} and R^{4d} are given in Table 1

Table CCXXIX provides 782 compounds of formula Igc 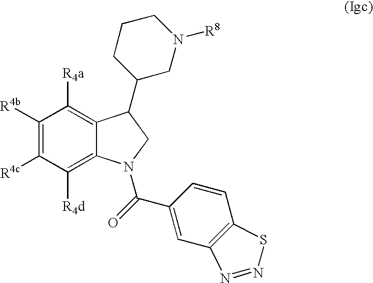
wherein the values of R⁸, R^{4a}, R^{4b}, R^{4c} and R^{4d} are given in Table 1

Table CCXXX provides 782 compounds of formula Igd 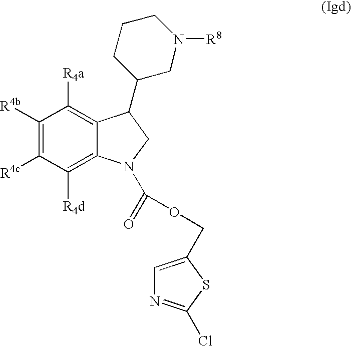
wherein the values of R⁸, R^{4a}, R^{4b}, R^{4c} and R^{4d} are given in Table 1

Table CCXXXI provides 782 compounds of formula Ige 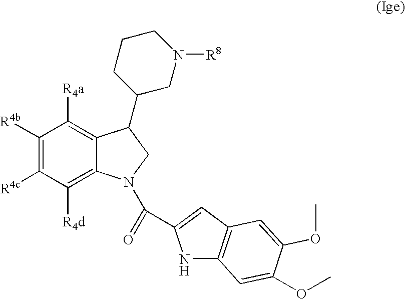
wherein the values of R⁸, R^{4a}, R^{4b}, R^{4c} and R^{4d} are given in Table 1

Table CCXXXII provides 782 compounds of formula Igf 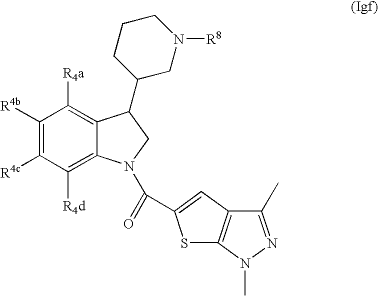
wherein the values of R⁸, R^{4a}, R^{4b}, R^{4c} and R^{4d} are given in Table 1

Table CCXXXIII provides 782 compounds of formula Igg 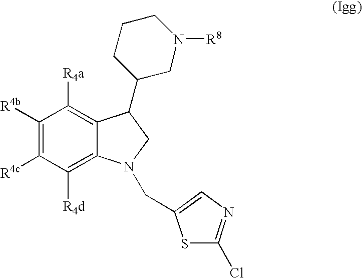
wherein the values of R⁸, R^{4a}, R^{4b}, R^{4c} and R^{4d} are given in Table 1

Table CCXXXIV provides 782 compounds of formula Igh 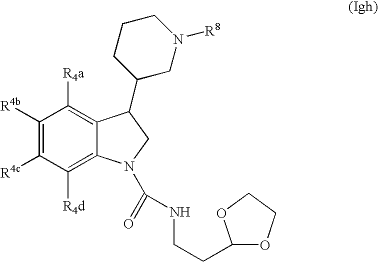
wherein the values of R⁸, R^{4a}, R^{4b}, R^{4c} and R^{4d} are given in Table 1

Table CCXXXV provides 782 compounds of formula Igi 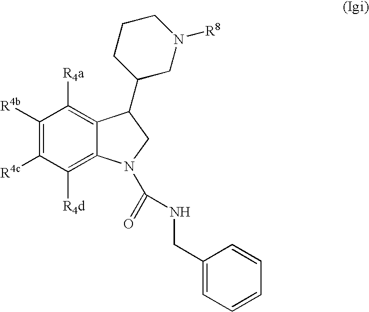
wherein the values of R⁸, R^{4a}, R^{4b}, R^{4c} and R^{4d} are given in Table 1

Table CCXXXVI provides 782 compounds of formula Igj 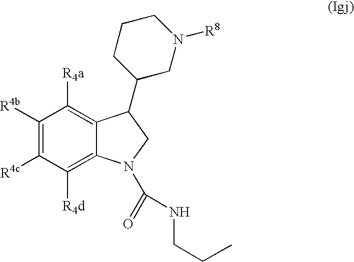
wherein the values of R⁸, R^{4a}, R^{4b}, R^{4c} and R^{4d} are given in Table 1

Table CCXXXVII provides 782 compounds of formula Igk 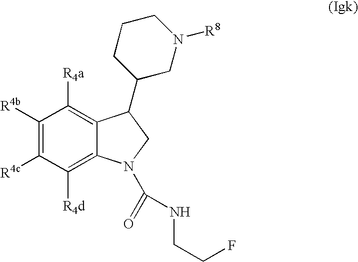
wherein the values of R⁸, R^{4a}, R^{4b}, R^{4c} and R^{4d} are given in Table 1

Table CCXXXVIII provides 782 compounds of formula Igl 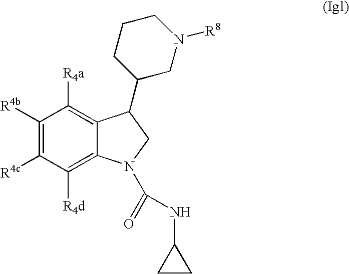
wherein the values of R⁸, R^{4a}, R^{4b}, R^{4c} and R^{4d} are given in Table 1

Table CCXXXIX provides 782 compounds of formula Igm 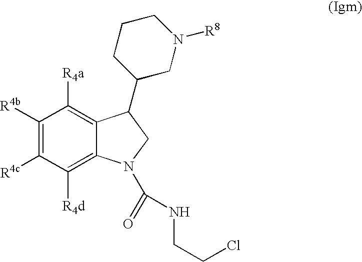
wherein the values of R⁸, R^{4a}, R^{4b}, R^{4c} and R^{4d} are given in Table 1

Table CCXL provides 782 compounds of formula Ign 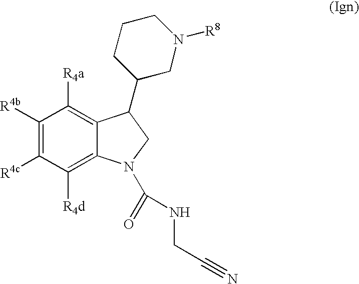
wherein the values of R⁸, R^{4a}, R^{4b}, R^{4c} and Re are given in Table 1

Table CCXLI provides 782 compounds of formula Igo 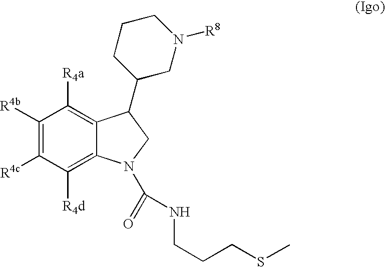
wherein the values of R⁸, R^{4a}, R^{4b}, R^{4c} and R^{4d} are given in Table 1

Table CCXLII provides 782 compounds of formula Igp 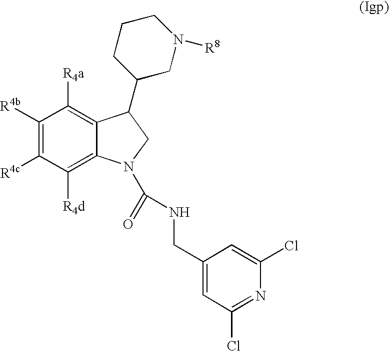
wherein the values of R⁸, R^{4a}, R^{4b}, R^{4c} and R^{4d} are given in Table 1

Table CCXLIII provides 782 compounds of formula Igq 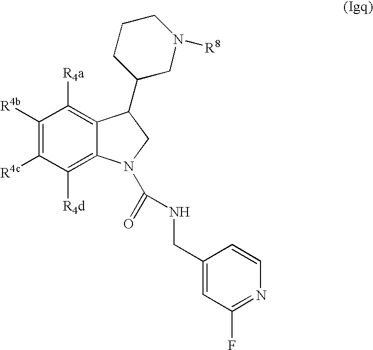
wherein the values of R⁸, R^{4a}, R^{4b}, R^{4c} and R^{4d} are given in Table 1.

Table CCXLIV provides 782 compounds of formula Igr 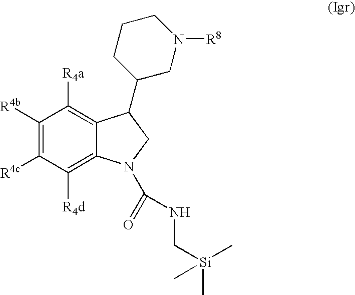
wherein the values of R⁸, R^{4a}, R^{4b}, R^{4c} and R^{4d} are given in Table 1.

Table CCXLV provides 782 compounds of formula Igs 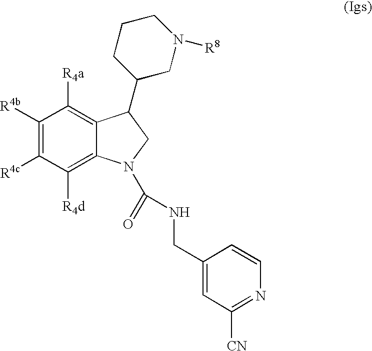
wherein the values of R⁸, R^{4a}, R^{4b}, R^{4c} and R^{4d} are given in Table 1.

Table CCXLVI provides 782 compounds of formula Igt 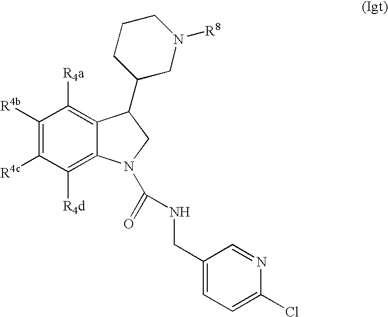
wherein the values of R⁸, R^{4a}, R^{4b}, R^{4c} and R^{4d} are given in Table 1.

Table CCXLVII provides 782 compounds of formula Igu 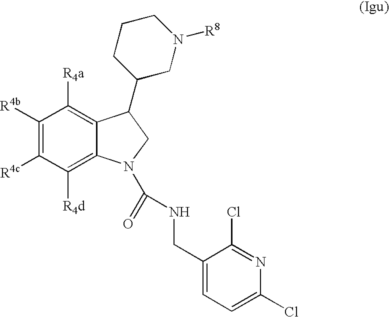
wherein the values of R⁸, R^{4a}, R^{4b}, R^{4c} and R^{4d} are given in Table 1.

Table CCXLVIII provides 782 compounds of formula Igv 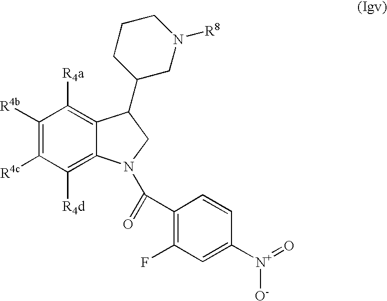
wherein the values of R⁸, R^{4a}, R^{4b}, R^{4c} and R^{4d} are given in Table 1.

Table CCXLIX provides 782 compounds of formula Igw 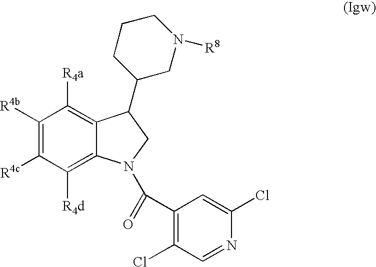
wherein the values of R⁸, R^{4a}, R^{4b}, R^{4c} and R^{4d} are given in Table 1.

Table CCL provides 782 compounds of formula Igx 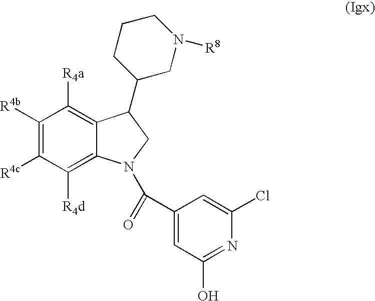
wherein the values of R⁵, R^{4a}, R^{4b}, R^{4c} and R^{4d} are given in Table 1.

Table CCLI provides 782 compounds of formula Igy 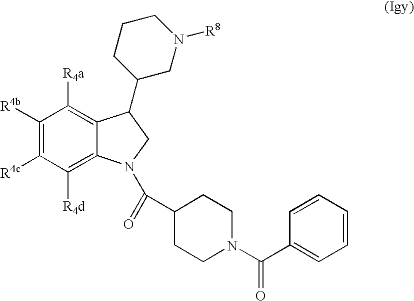
wherein the values of R⁸, R^{4a}, R^{4b}, R^{4c} and R^{4d} are given in Table 1.

Table CCLII provides 782 compounds of formula Igz 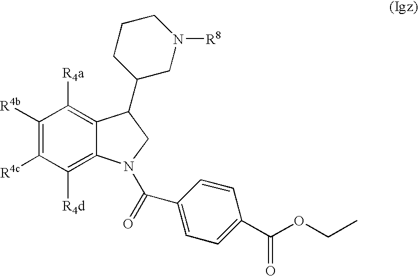
wherein the values of R⁸, R^{4a}, R^{4b}, R^{4c} and R^{4d} are given in Table 1.

Table CCLIII provides 782 compounds of formula Iha 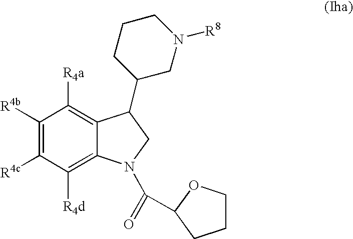
wherein the values of R⁸, R^{4a}, R^{4b}, R^{4c} and R^{4d} are given in Table 1.

Table CCLIV provides 782 compounds of formula Ihb 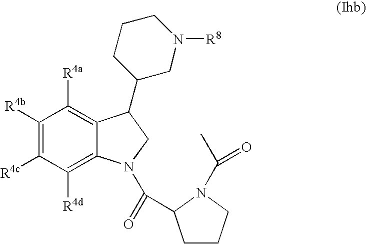
wherein the values of R⁸, R^{4a}, R^{4b}, R^{4c} and R^{4d} are given in Table 1.

Table CCLV provides 782 compounds of formula Ihc 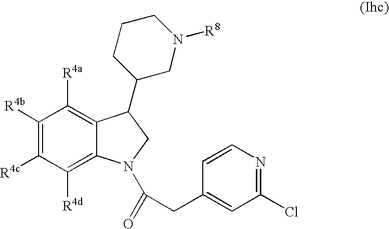
wherein the values of R⁸, R^{4a}, R^{4b}, R^{4c} and R^{4d} are given in Table 1.

Table CCLVI provides 782 compounds of formula Ihd 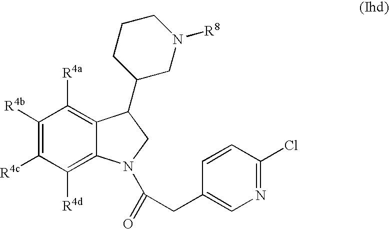
wherein the values of R⁸, R^{4a}, R^{4b}, R^{4c} and R^{4d} are given in Table 1.

Table CCLVII provides 782 compounds of formula Ihe 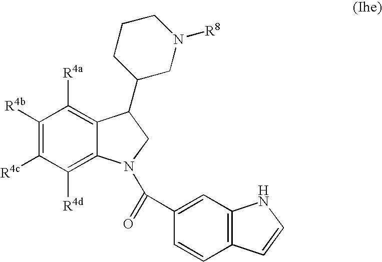
wherein the values of R⁸, R^{4a}, R^{4b}, R^{4c} and R^{4d} are given in Table 1.

Table CCLVIII provides 782 compounds of formula Ihf 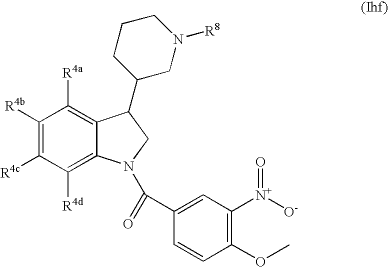
wherein the values of R⁸, R^{4a}, R^{4b}, R^{4c} and R^{4d} are given in Table 1.
Table CCLIX provides 782 compounds of formula Ihg 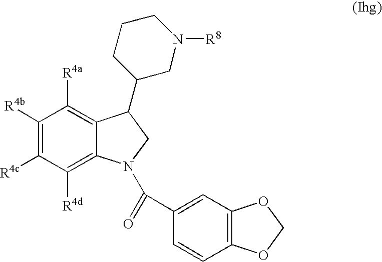
wherein the values of R⁸, R^{4a}, R^{4b}, R^{4c} and Re are given in Table 1.

Table CCLX provides 782 compounds of formula Ihh 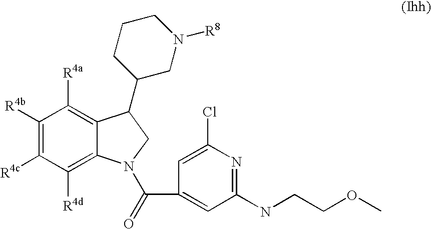
wherein the values of R⁸, R^{4a}, R^{4b}, R^{4c} and R^{4d} are given in Table 1.

Table CCLXI provides 782 compounds of formula Ihi 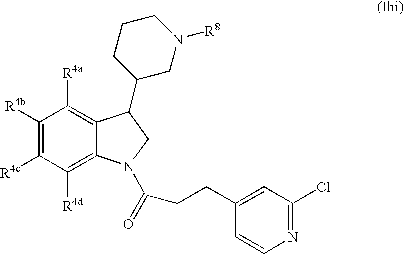
wherein the values of R⁸, R^{4a}, R^{4b}, R^{4c} and R^{4d} are given in Table 1.

Table CCLXII provides 782 compounds of formula Ihj 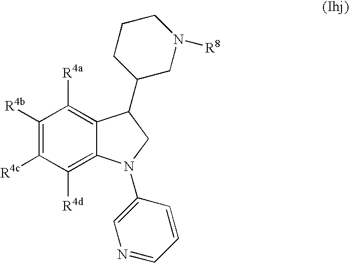
wherein the values of R⁸, R^{4a}, R^{4b}, R^{4c} and R^{4d} are given in Table 1.

Table CCLXIII provides 782 compounds of formula Ihk 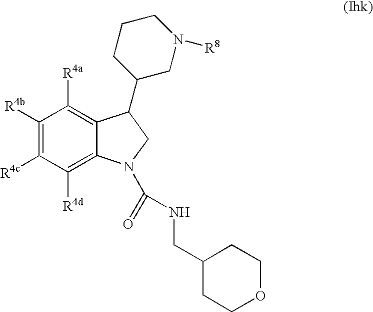
wherein the values of R⁸, R^{4a}, R^{4b}, R^{4c} and R^{4d} are given in Table 1.

Table CCLXIV provides 782 compounds of formula Ihl 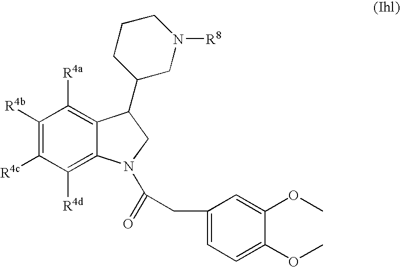
wherein the values of R⁸, R^{4a}, R^{4b}, R^{4c} and R^{4d} are given in Table 1.

Table CCLXV provides 782 compounds of formula Ihm 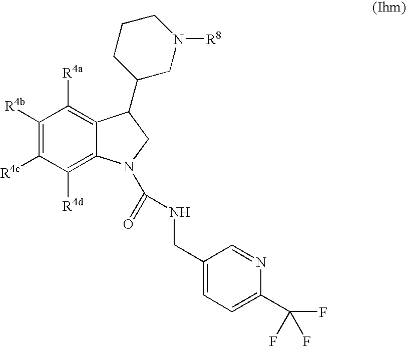
wherein the values of R⁸, R^{4a}, R^{4b}, R^{4c} and R^{4d} are given in Table 1.

Table CCLXVI provides 782 compounds of formula Ihn 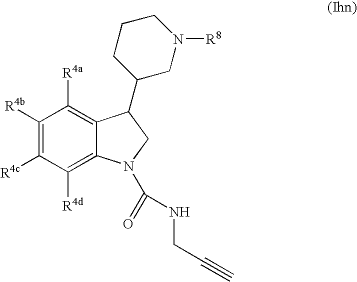
wherein the values of R⁸, R^{4a}, R^{4b}, R^{4c} and R^{4d} are given in Table 1.

Table CCLXVII provides 782 compounds of formula Iho 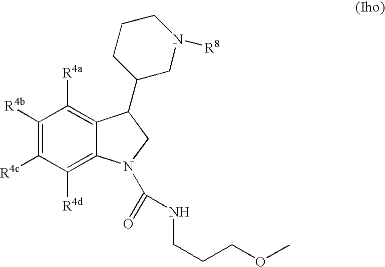
wherein the values of R⁸, R^{4a}, R^{4b}, R^{4c} and R^{4d} are given in Table 1.

Table CCLXVIII provides 782 compounds of formula Ihp

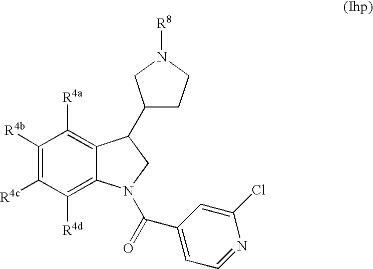

wherein the values of R⁸, R^{4a}, R^{4b}, R^{4c} and R^{4d} are given in Table 2.

TABLE 1

| Compound | | | | | |
|---|---|---|---|---|---|
| No | R⁸ | R^{4a} | R^{4b} | R^{4c} | R^{4d} |
| CCXVIII-1 | 4-chlorocinnamyl | CH₃ | H | H | H |
| | | diasteroisomerA | | | |
| CCLXVIII-2 | 4-chlorocinnamyl | H | H | H | CH₃ |
| CCLXVIII-3 | 4-chlorocinnamyl | H | H | CH₃ | H |
| CCLXVIII-4 | 4-chlorocinnamyl | CH₃ | H | H | H |
| | | diasteroisomerB | | | |

Mass spectra data were obtained for selected compounds of Tables I to CCLXVIII using LCMS: LC5: 254 nm—gradient 10% A to 100% B A=H2O+0.01% HCOOH B═CH3CN/CH3OH+0.01% HCOOH positive electrospray 150-1000 m/z.

The data are shown in Table 2:

TABLE 2

| | LCMS | MS data |
|---|---|---|
| Compound | (Ret. Time, min) | (M + H⁺) |
| III.3 | 2′21 | 492 |
| III.26 | 2′24 | 510 |
| III.49 | 2′36 | 526 |
| III.118 | 2′14 | 522 |
| LXXI.3 | 2′21 | 492 |
| LXXI.26 | 1′42/1′50 | 496 |
| LXXI.29 | 1′6 | 530 |
| LXXI.49 | 1′32/1′38 | 512 |
| LXXI.348 | 1′58 | 498 |
| LXXIII.26 | 1′67 | 532 |
| LXXVI.26 | 1′55 | 480 |
| CCLXVIII-1 | 1′6 | 492 |
| CCLXVIII-2 | 1′6 | 492 |
| CCLXVIII-3 | 1′6 | 492 |
| CCLXVIII-4 | 1′59 | 492 |

The compounds of this invention may be prepared by a variety of methods and some are illustrated in the following Schemes. For example compounds of formula 1 may be synthesised as described on Scheme 1.

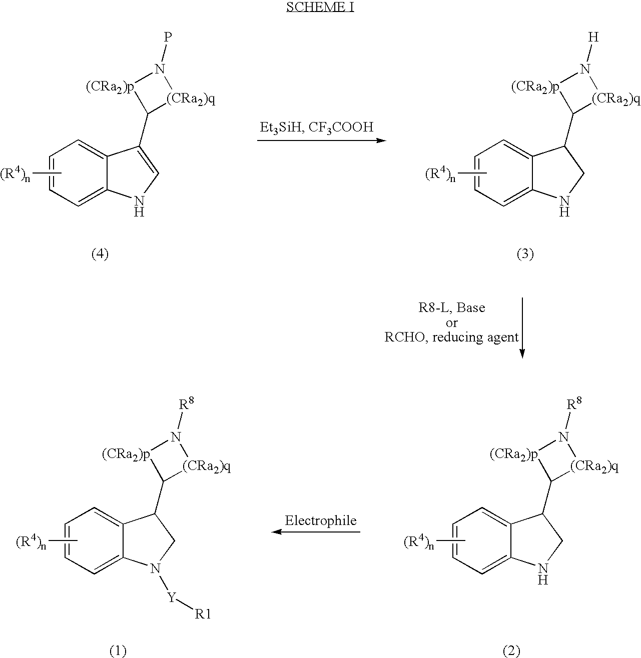

A compound of formula 1 may be synthesised from a compound of formula 2 by reaction with a suitable electrophilic species. Compounds of formula 1 where Y is a carbonyl group may be formed with a carboxylic acid derivative of formula R1—C(O)-Z where Z is chloride, hydroxy, alkoxy or acyloxy at a temperature between 0° C. and 150° C. optionally in an organic solvent such as dichloromethane, chloroform or 1,2-dichloroethane, optionally in the presence of a tertiary amine base such as triethylamine or diisopropylethylamine and optionally in the presence of a coupling agent such as dicyclohexylcarbodiimide. Compounds of formula 1 where Y is a carbonyl group and R1 is an amino substituent of formula R′—NH—may be formed by the reaction of compounds of formula 2 with an isocyanate of formula R′—N—C═O under similar conditions. Compounds of formula 1 where Y is a group of formula S(O)_{q} may be formed from compounds of formula 6 by treatment with compounds of formula of R1—S(O)_{q}—Cl under similar conditions. Compounds of formula 1 where Y is a thiocarbonyl group and RI is an amino substituent of formula R′—NH— may be formed by the reaction of compounds of formula 2 with an isothiocyanate of formula R′—N═C═S under similar conditions. Alternatively compounds of formula 1 where Y is a thiocarbonyl group and R1 is a carbon substituent may be formed by treatment of compounds of formula 1 where Y is a carbonyl group and RI is a carbon substituent with a suitable thionating agent such as Lawesson's reagent.

In the above procedures, acid derivatives of the formula R1—C(O)-Z, isocyanates of formula R′—N═C═O, isothiocyanates of formula R′—N═C═S and sulfur electrophiles of formula R1—S(O)_{q}—Cl are either-known compounds or may be formed from known compounds by known methods by a person skilled in the art.

A compound of formula 2 may be synthesised from a compound of formula 3 by reaction by the reaction with an alkylating agent of the formula R8-L, where L is chloride, bromide, iodide or a sulfonate (e.g. mesylate or tosylate) or similar leaving group at a temperature of between ambient temperature and 100° C., typically ambient temperature, in an organic solvent such as acetonitrile, dimethylformamide, dichloromethane, chloroform or 1,2-dichloroethane in the presence of a tertiary amine base such as triethylamine or diisopropylethylamine and optionally catalysed by halide salts such as sodium iodide, potassium iodide or tetrabutylammonium iodide.

Certain compounds of formula 2 are novel and as such form a further aspect of the invention.

Alternatively a compound of formula 3 may be reacted with an aldehyde of the formula RCHO at a temperature between ambient temperature and 100° C. in an organic solvent such as tetrahydrofuran or ethanol or mixtures of solvents in the presence of a reducing agent such as borane-pyridine complex, sodium borohydride, sodium (triacetoxy)borohydride, sodium cyanoborohydride or such like, to produce a compound of formula 2 where R8 is CH₂—R.

Compounds of formula 3 may be synthesised from a compound of formula 4 wherein P is H or a protecting group such as tert-butoxycarbonyl by reduction of the indolinic double bond with a reducing agent such as triethylsilane, borane or lithium aluminium hydride in the presence of an acid such as trifluoroacetic acid in an organic solvent such as dichloromethane or chloroform or without solvent at a temperature of between 0° C. to 100° C.

Compounds of formula 4 may be prepared by known methods by the person skilled in the art.

For example, compounds of formula 4 may be synthesised from an indene of formula 5 following the procedure disclosed in Tetrahedron 2001, 57, 2039-2049 (Scheme 2).

Indenes of formula 5 are either known compounds or may be prepared by known methods by the person skilled in the art.

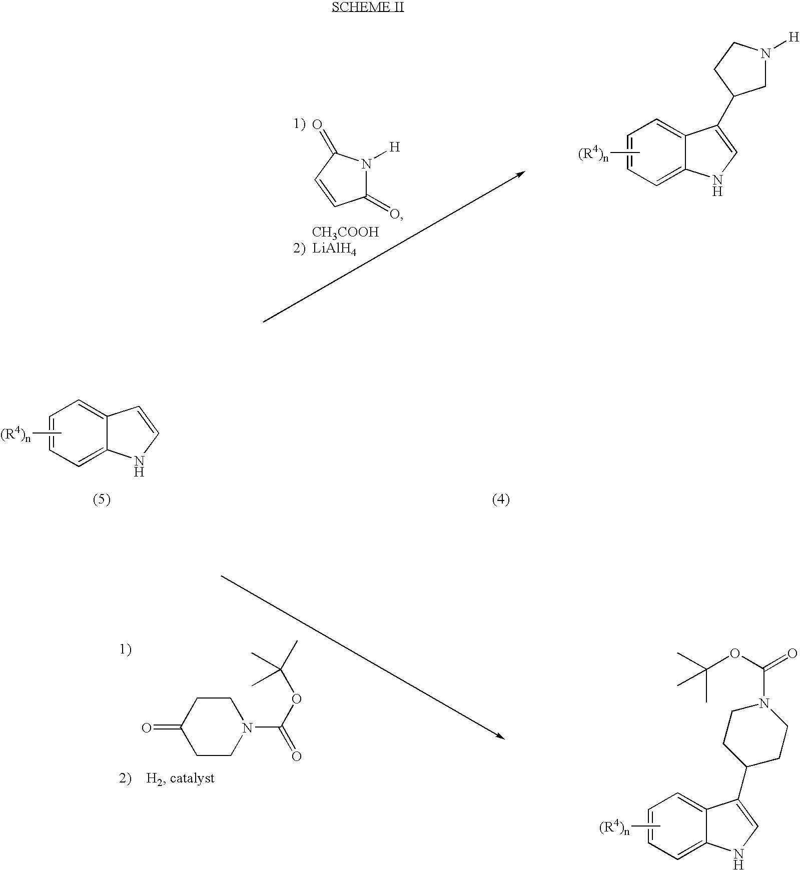

Alternatively, compounds of formula 1 may be synthesised as described on Scheme 3.

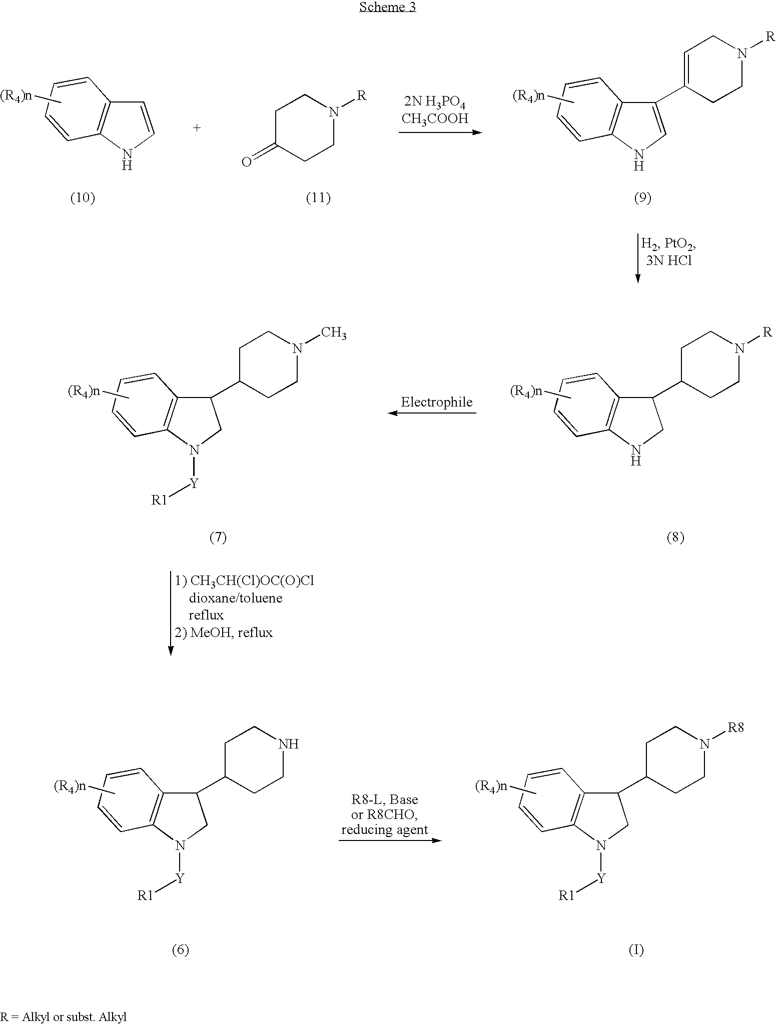

Alternatively, compounds of formula 1 may be synthesised as described on Scheme 4.

Compounds of formula 1 where R² and R³ are other than hydrogen may be made by the routes of Scheme 4. Thus for example a compound of formula 16a which are compounds of formula 16 wherein the carbon atom adjacent to the OH group carries groups R² and R³ may be converted to compounds of formula I using the methods for converting compounds of formula 16 into compounds of formula I.

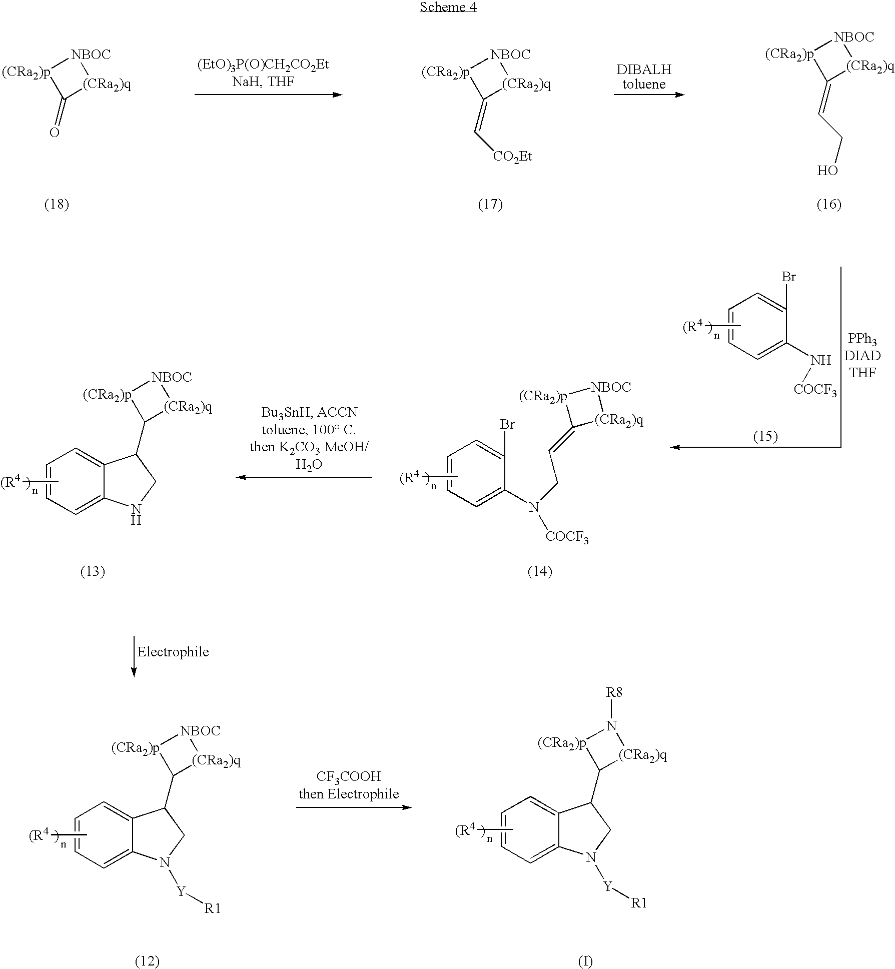

The compounds of formula (I) can be used to combat and control infestations of insect pests such as Lepidoptera, Diptera, Heriptera, Thysanoptera, Orthoptera, Dictyoptera, Coleoptera, Siphonaptera, Hymenoptera and Isoptera and also other invertebrate pests, for example, acarine, nematode and mollusc pests. Insects, acarines, nematodes and molluscs are hereinafter collectively referred to as pests. The pests which may be combated and controlled by the use of the invention compounds include those pests associated with agriculture (which term includes the growing of crops for food and fibre products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies).

Examples of pest species which may be controlled by the compounds of formula (I) include: Myzus persicae (aphid), Aphis gossypii (aphid), Aphis fabae (aphid), Lygus spp. (capsids), Dysdercus spp. (capsids), Nilapaivata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Benzisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Pluteilla xylostella (diamond back moth), Agrotis spp. (cutworms), Chilo suppressalis (rice stem borer), Locusta migratoria (locust), Chlortiocetes terminifera (locust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalidesfelis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes jormosanus, Reticulitermes flavipes, R. speratu, R. virginicus, R. hesperus, and R. santonensis) and the Termitidae (for example Globitermes sulphureus), Solenopsis geminata (fire ant), Monomorium pharaonis (pharaoh's ant), Damalinia spp. and Linognathus spp. (biting and sucking lice), Meloidogyne spp. (root knot nematodes), Globodera spp. and Heterodera spp. (cyst nematodes), Pratylenchus spp. (lesion nematodes), Rhodopholus spp. (banana burrowing nematodes), Tylenchulus spp.(citrus nematodes), Haemonchus contortus (barber pole worm), Caenorhabditis elegans (vinegar eelworm), Trichostrongylus spp. (gastro intestinal nematodes) and Deroceras reticulatuin (slug).

The invention therefore provides a method of combating and controlling insects, acarines, nematodes or molluscs which comprises applying an insecticidally, acaricidally, nematicidally or molluscicidally effective amount of a compound of formula (I), or a composition containing a compound of formula (I), to a pest, a locus of pest, or to a plant susceptible to attack by a pest, The compounds of formula (I) are preferably used against insects, acarines or nematodes.

The term “plant” assessed herein includes seedlings, bushes and trees.

In order to apply a compound of formula (I) as an insecticide, acaricide, nematicide or molluscicide to a pest, a locus of pest, or to a plant susceptible to attack by a pest, a compound of formula (I) is usually formulated into a composition which includes, in addition to the compound of formula (I), a suitable inert diluent or carrier and, optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably I to 85%, for example 5 to 60%, of a compound of formula (I). The composition is generally used for the control of pests such that a compound of formula (I) is applied at a rate of from 0.1 g to 10 kg per hectare, preferably from 1 g to 6 kg per hectare, more preferably from 1 g to 1 kg per hectare.

When used in a seed dressing, a compound of formula (I) is used at a rate of 0.0001 g to log (for example 0.001 g or 0.05 g), preferably 0.005 g to log, more preferably 0.005 g to 4 g, per kilogram of seed.

In another aspect the present invention provides an insecticidal, acaricidal, nematicidal or molluscicidal composition comprising an insecticidally, acaricidally, nematicidally or molluscicidally effective amount of a compound of formula (I) and a, suitable carrier or diluent therefor. The composition is preferably an insecticidal, acaricidal, nematicidal or molluscicidal composition.

In a still further aspect the invention provides a method of combating and controlling pests at a locus which comprises treating the pests or the locus of the pests with an insectici dally, acaricidally, nematicid ally or molluscicidally effective amount of a composition comprising a compound of formula (I). The compounds of formula (I) are preferably used against insects, acarines or nematodes.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) (or a solution thereof, in a suitable agent) in a porous granular material (such as pumice, attapulgite clays, fuller's earth, kieselguhr, diatomaceous earths or ground corn cobs) or by adsorbing a compound of formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at room temperature, it may be melted at a reasonable temperature, typically below 70° C.) or in solution (by dissolving it in an appropriate solvent) and then emulsiflying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula (I) is present initially in either the water or the solvent/SFA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I). SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (I) may be dry milled and added to water, containing agents hereinbefore described, to produce the desired end product.

Aerosol formulations comprise a compound of formula (I) and a suitable propellant (for example n-butane). A compound of formula (I) may also be dissolved or dispersed in a suitable medium (for example water or a water miscible liquid, such as n-propanol) to provide compositions for use in non-pressurised, hand-actuated spray pumps.

A compound of formula (I) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula (I) and, optionally, a carrier or diluent therefor. The polymeric shell may be produced by either an interfacial polycondensation reaction or by a coacervation procedure. The compositions may provide for controlled release of the compound of formula (I) and they may be used for seed treatment. A compound of formula (I) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (I)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (I)).

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-estets), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (I) may be applied by any of the known means of applying pesticidal compounds. For example, it may be applied, formulated or unformulated, to the pests or to a locus of the pests (such as a-habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion-of the active ingredient, the concentrate being added to water before use. These concentrates, which may include DCs, SCs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

A compound of formula (I) may be used in mixtures with fertilisers (for example nitrogen-, potassium- or phosphorus-containing fertilisers). Suitable formulation types include granules of fertiliser. The mixtures suitably contain up to 25% by weight of the compound of formula (I).

The invention therefore also provides a fertiliser composition comprising a fertiliser and a compound of formula (I).

The compositions of this invention may contain other. compounds having biological activity, for example micronutrients or compounds having fungicidal activity or which possess plant growth regulating, herbicidal, insecticidal, nematicidal or acaricidal activity.

The compound of formula (I) may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergise the activity or complement the activity (for example by increasing the speed of effect or overcoming repellency) of the compound of formula (I); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin), bifenthrin, fenpropathrin, cyfluthrin, tefluthrin, fish safe pyrethroids (for example ethofenprox), natural pyrethrin, tetramethrin, s-bioallethrin, fenfluthrin, prallethrin or 5-benzyl-3-furylmethyl-(E)-(1R,3S)-2,2-dimethyl-3-(2-oxothiolan-3-ylidenemethyl)cyclopropane carboxylate;

b) Organophosphates, such as, profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;

c) Carbamates (including aryl carbamates), such as pinimicarb, triazamate, cloethocarb, carbofuran, furathiocarb, ethiofencarb, aldicarb, thiofurox, carbosulfan, bendiocarb, fenobucarb, propoxur, methomyl or oxamyl;

d) Benzoyl ureas, such as diflubenzuron, triflumuron, hexaflumuron, flufenoxuron or chlorfluazuron;

e) Organic tin compounds, such as cyhexatin, fenbutatin oxide or azocyclotin;

f) Pyrazoles, such as tebufenpyrad and fenpyroximate;

g) Macrolides, such as avermectins or milbemycins, for example abamectin, emamectin benzoate, ivermectin, milbemycin, spinosad or azadirachtin;

h) Hormones or pheromones;

i) Organochlorine compounds such as endosulfan, benzene hexachloride, DDT, chlordane or dieldrin;

j) Amidines, such as chlordimeform or amitraz;

k) Fumigant agents, such as chloropicrin, dichloropropane, methyl bromide or metam;

l) Chloronicotinyl compounds such as imidacloprid, thiacloprid, acetamiprid, nitenpyram or thiamethoxam;

m) Diacylhydrazines, such as tebufenozide, chromafenozide or methoxyfenozide;

n) Diphenyl ethers, such as diofenolan or pyriproxifen;

o) Indoxacarb;

p) Chlorfenapyr; or

q) Pymetrozine.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulphonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)-4methylbenzamide (RH-7281, zoxamide), N-allyl4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704), alanycarb, aldirnorph, anilazine, azaconazole, azoxystrobin, benalaxyl, benomyl, biloxazol, bitertanol, blasticidin S, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulphate, copper tallate and Bordeaux mixture, cymoxanil, cyproconazole, cyprodinil, debacarb, di-2-pyridyl disulphide 1,1′-dioxide, dichlofluanid, diclomezine, dicloran, diethofencarb, difenoconazole, difenzoquat, diflumetorim, O,O-di-iso-propyl-S-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl(Z)-N-benzyl-A(Cmethyl(methyl-thioethylideneaminooxycarbonyI)amino]tho)-p -alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluoroimide, fluquinconazole, flusilazole, flutolanil, flutriafol, folpet, fuberidazole, fliralaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, kasugamycin, kresoxim-methyl, LY186054, LY211795, LY248908, rnancozeb, maneb, mefenoxam, mepanipyrim, mepronil, metalaxyl, metconazole, metiram, metiram-zinc, metominostrobin, myclobutanil, neoasozin, nickel dimethyldithiocarbarnate, nitrothal-isopropyl, nuarimol, ofurace, organomercury compounds, oxadixyl, oxasuilfron, oxolinic acid, oxpoconazole, oxycarboxin, pefurazoate, penconazole, pencycuron, phenazin oxide, phosetyl-Al, phosphorus acids, phthalide, picoxystrobin (ZA1963), polyoxin D, polyram, probenazole, prochloraz, procymidone, propamocarb, propiconazole, propineb, propionic acid, pyrazophbs, pyrifenox, pyrimethanil, pyroquilon, pyroxyfur, pyrrolnitrin, quatemary ammonium compounds, quinomethionate, quinoxyfen, quintozene, sipconazole (F-155), sodium pentachlorophenate, spiroxamine, streptomycin, sulphur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tQlyflluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram.

The compounds of formula (I) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIXTM.

Some mixtures may comprise active ingredients which have significantly different physical, chemical or biological properties such that they do not easily lend themselves to the same conventional formulation type. In these circumstances other formulation types maybe prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The invention is illustrated by the following Examples:

### EXAMPLE 1

This Example illustrates the preparation of compound III.49, (5-Chloro-3-{1-[(E)-3-(4-chloro-phenyl)-propenyl]-piperidin-4-yl)-2,3-dihydro-indol-1-yl)-(2-chloro-pyridin-4-yl)-methanone

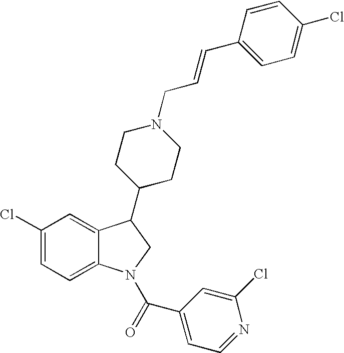

Step A: By analogy to the procedure described by P. Gharagozloo in Tetrahedron 1996, 52, 10185-10192, 5-chloroindole (0.42 g) and N-tert-butoxycarbonyl-piperidin-4-one (1.65 g) were dissolved in methanol (7 ml) and a suspension of sodium methoxide (0.9 g) in methanol (15 ml) was added dropwise to this solution. The resulting mixture was refluxed for 40 hours, cooled to room temperature, quenched by addition of acetic acid (0.8 ml) and concentrated in vacuo. The residue was partitioned between dichloromethane and saturated aqueous sodium bicarbonate, extracted four times with dichloromethane, washed with brine, dried (sodium sulphate) and concentrated in vacuo to afford 4-(5-Chloro-1H-indol-3-yl)-3,6-dihydro-2H-pyridine-1-carboxylic acid tert-butyl ester (0,5 g); ¹H NMR (300 MHz, CDCl₃) 1.50 (s, 9H), 2.46 (m, 2H), 3.61 (m, 2H), 4.03 (m, 2H), 6.02 (m, 1H), 7.03-7.22 (m, 3H), 7.77 (s, 1H), 8.43 (brs, 1H); MS (ES+) 277/279 (-isoprene+H⁺).

Step B: The product obtained in Step A (0.5 g) was hydrogenated in tetrahydrofuran (10 ml) under 1 atmosphere in the presence of 5% Rh/C (25 mg) to afford after standard work-up and silica gel chromatography (cyclohexane: ethyl acetate 75:25) 4-(5-Chloro-1H-indol-3-yl)-piperidine-1-carboxylic acid tert-butyl ester (0.15 g); ¹H NMR (400 MHz, CDCl₃) 1.49 (s, 9H), 1.55 (m, 2H), 1.92 (m, 2H), 2.70 (m, 2H), 4.18 (m, 2H), 6.71 (s, 1H), 7.05 (d, 1H), 7.16 (d, 1H), 7.50 (s, 1H), 8.43 (brs, 1H); MS.(ES+) 235/237 (M-isoprene-CO₂+H⁺).

Step C: The product obtained in Step B (0.15 g) was dissolved in trifluoroacetic acid (7.5 ml) and treated with triethylsilane (0.21 ml). The solution was stirred at 65° C. for 6 hours, cooled to room temperature and poured into cold 10% aqueous sodium hydroxide (80 ml). The mixture was extracted four times with dichloromethane, washed with brine, dried (sodium sulphate) and concentrated to afford a crude residue containing mostly 5-chloro-3-piperidin-4-yl-2,3-dihydro-1H-indole (0.19 g). This residue was dissolved in acetonitrile (3 ml) and alkylated with 4-chlorocinnamyl chloride (78 mg) in the presence of diisopropylethylamine (0.12 g) for 4 hours at room temperature. The solution was concentrated in vacuo and the residue purified by silica gel chromatography (eluent ethyl acetate/methanol 9:1) to afford 5-Chloro-3-{1-[(E)-3-(4-chloro-phenyl)-allyl]-piperidin-4-yl}-2,3-dihydro-1H-indole (0.12 g) as an oil; MS (ES+) 387/389 (M+H⁺). This compound was dissolved in dichloromethane (3 ml) and acylated with 2-chloro-isonicotinoyl chloride (0.13 g) in the presence of triethylamine (0.1 6 ml) at 0° C. for 2 hours. Standard aqueous work-up and silica gel chromatography (ethyl acetate/methanol 95:5) afforded the title product (60 mg). MS (ES+) 526/528 (M+H⁺). Compounds III.3 and III.26 were prepared according to procedures analogous to those described in Example 1.

### EXAMPLE 2

This Example illustrates the preparation of compound LXXI.26, 3-{1-[(E)-3-(4-Chloro-phenyl)-allyl]-pyrrolidin-3-yl}-5-fluoro-2,3-dihydro-indol-1-yl)-(2-chloro-pyridin-4-yl)-methanone

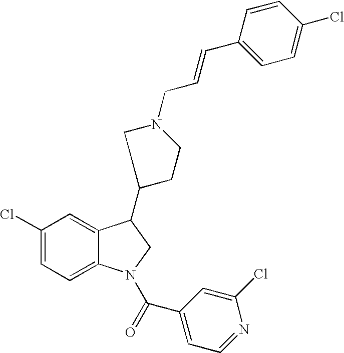

Step A: 3-(5-Fluoro-1H-indol-3-yl)-pyrrolidine-2,5-dione (0.35 g) (prepared by mixing 5-fluoroindole (0.75 g) and maleimide (1.6 g) in acetic acid (30 ml) for 24 hours at reflux, as described by J. E. Macor in Synthesis 1997, 443) was reduced with lithium aluminium hydride (0.29 g) in tetrahydrofuran (15 ml) for 4 hours at reflux. The reaction mixture was cooled to 0° C., quenched by careful addition of water (0.27 ml), 15% NaOH (0.27 ml) then water (0,8 ml). The mixture was diluted with ethyl acetate, stirred for 12 hours at room temperature, filtered and the solvents were removed in vacuo to afford crude 5-fluoro-3-pyrrolidin-3-yl-1H-indole (0.36 g), which was used as such for the next step. MS (ES+) 205 (M+H⁺).

Step B: 5-Fluoro-3-pyrrolidin-3-yl-1H-indole obtained in step A (0.18 g) was dissolved in trifluoroacetic acid (8 ml) and treated with triethylsilane (0.4 ml) at 55° C. for 5 hours. The dark solution was poured into cold 10% aqueous sodium hydroxide, extracted three times with dichloromethane, washed with brine, dried (sodium sulphate) and concentrated to afford a crude residue (0.16g) containing mostly 5-fluoro-3-pyrrolidin-3-yl-2,3-dihydro-1H-indole. The latter was dissolved in acetonitrile (1.5 ml) and alkylated with 4-chlorocinnamyl chloride (0.1 1 g) in the presence of diisopropylethylamine (0.19 g) for 2 hours at room temperature. The solution was concentrated in vacuo and the residue purified by silica gel chromatography (eluent ethyl acetatelmethanol 8:2) to afford 3-{1-[(E)-3-(4-Chloro-phenyl)-allyl]-pyrrolidin-3-yl}-5-fluoro-2,3-dihydro-1H-indole as a mixture of diastereoisomers; MS (ES+) 357/359 (M+H⁺). This compound was dissolved in dichloromethane (5 ml) and acylated with 2-chloroisonicotinoyl chloride (49 mg) in the presence of triethylamine (0.1 ml) at 0° C. for 2 hours. Standard aqueous work-up and silica gel chromatography (ethyl acetate/methanol 95:5) afforded the title product as a mixture of diastereoisomers (50 mg). MS (ES+) 496/498 (M+H⁺).

Compounds LXXI.3 and LXXI.49 were prepared according to procedures analogous to those described in Example 2.

### EXAMPLE 3

This example illustrates the preparation of compound III.118

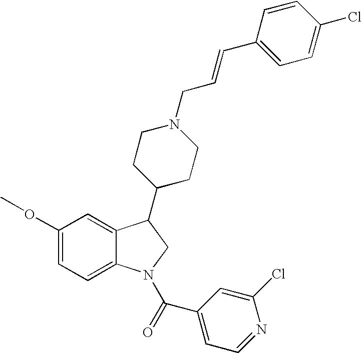

Step A: Synthesis of intermediate A

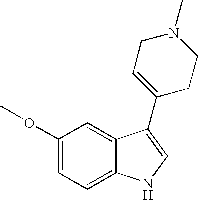

5-Methoxyindole (3.0 g) was dissolved in 60 ml of glacial acetic acid and heated to 115° C. Then 15 ml of 2N phosphoric acid and 1-methyl-4-piperidone (14.1 ml) was added. The mixture was stirred at this temperature for 2 h, then poured on ice-ammonia and the reaction products were extracted with ethyl acetate. Silica gel chromatography (ethyl acetate/methanol/Et₃N 90:5:5) afforded the intermediate A (1.62 g). MS (ES+) 243 (M+H⁺).

Step B: Synthesis of intermediate B

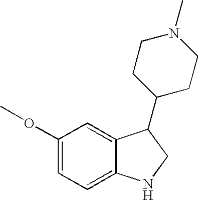

Hydrogenation of intermediate A (2.0 g) in 2N HCl (60 ml) with PtO₂ (0.4 g) as catalyst afforded 1.65 g of intermediate B. MS (ES+) 247 (M+H⁺). Step C: Synthesis of intermediate C

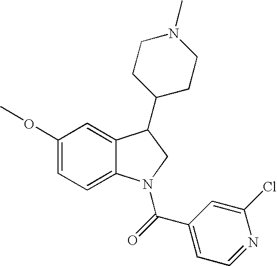

Intermediate B (615 mg) was dissolved in dichloromethane (20 ml) and acylated with 2-chloroisonicotinoyl chloride (1.5 eq.) in the presence of triethylamine (0.87 ml) at 0° C. for 3 hours. Standard aqueous work-up and silica gel chromatography (ethyl acetate/methanol 8:2) afforded intermediate C (662 mg). MS (ES+) 386 (M+H⁺). Step D: Synthesis of intermediates D and E

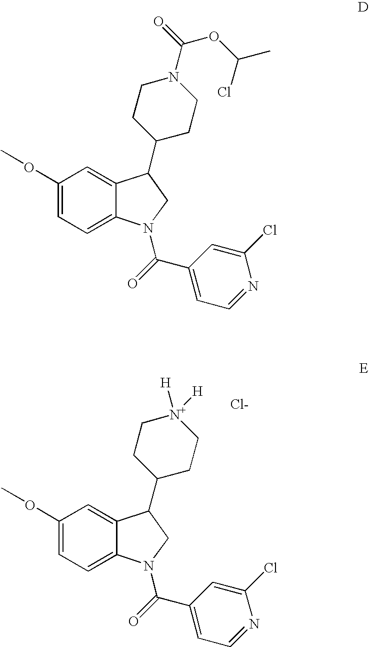

Intermediate C (384 mg) was dissolved in toluene (25 ml) and dioxane (10 ml), cooled to 0° C. and treated with 1-cbloroethylchloroformate (2.18 ml): The reaction mixture was then refluxed for 16 h, poured onto saturated aqueous NaHCO₃ solution and extracted with dichloromethane. Evaporation afforded 380 mg of crude intermediate D.

Intermediate D (380 mg) was dissolved in methanol (20 ml) and refluxed for 16 h. Evaporation of the reaction mixture under reduced pressure afforded intermediate E (200 mg). MS (ES+) 372 (M−Cl⁻). Step E: Synthesis of target compound III.118 Intermediate E (200 mg) was dissolved in chloroform (15 ml) and reacted with 4-chlorocinnamyl chloride (98 mg) in the presence of triethylamine (0.208 ml) for 16 h at 65° C. The reaction mixture was concentrated under reduced pressure and the residue purified by silica gel chromatography with dichloromethane/methanol/triethylamine 98:1.9:0.1 as eluent to afford the title compound (75 mg) as highly viscous oil. MS (ES+) 522 (M+H⁺).

### EXAMPLE 4

This Example illustrates the preparation of compound CCIII.26 (5-fluoro-3-{1-[(E)-3-(4-chloro-phenyl)-propenyl]-piperidin-3-yl}-2,3-dihydro-indol- 1 -yl)-(2-chloro-pyridin-4-yl)-methanone

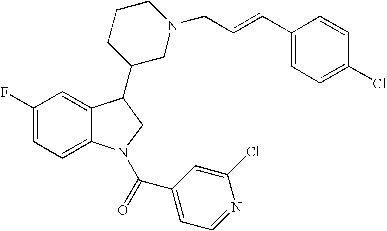

Step A: ethyl triethylphosphonoacetate (6.7 ml) in tetrahydrofuran (14 ml) was added dropwise to a suspension of sodium hydride (60% in oil, 1.3 g) in tetrahydrofiran (36 ml) at 0° C. under argon and the resulting mixture was stirred 30 min at 0° C. 1-BOC-3-piperidone (6 g) in tetrahydrofuran (14 ml) was added dropwise at 0° C. and the resulting solution was stirred at room temperature for 1 hour. The solvent was removed in vacuo, the residue partitioned between dichloromethane and water and the aqueous layer extracted four times with dichloromethane. The combined organic layers were washed with diluted aqueous sodium carbonate, dried over sodium sulfate and concentrated in vacuo. Filtration on silica gel afforded 3-[1 -ethoxycarbonyl-meth-(E)-ylidene]-piperidine-1-carboxylic acid tert-butyl ester (7.6 g, mixture of diastereoisomers) as an oil, which was characterised by its mass and NMR spectra.

Step B: a solution of the product obtained in Step A (7.6 g) in toluene (57 ml) at 5° C. was treated with diisobutylaluminium hydride (1.6M in toluene, 56 ml) and the resulting solution was stirred at room temperature for 1.5 hour. The reaction was quenched by careful addition of 1N HCl (90 ml), then basified with 1N NaOH. The mixture was filtered over Hyflo, extracted two times with ethyl acetate; the combined organic layers were washed with saturated aqueous sodium bicarbonate then water, dried over sodium sulfate and concentrated in vacuo. The residue purified by silica gel chromatography (eluent ethyl acetate/cyclohexane 4:6) to afford 3-[2-hydroxy-eth-(E)-ylidene]-piperidine-1-carboxylic acid tert-butyl ester (5 g) as an oil, which was characterised by its NMR spectrum.

Step C: diisopiopylazodicarboxylate (4.2 ml) was added dropwise to a solution of triphenylphosphine (3.8 g) in tetrahydrofuran (80 ml) at −10° C. under argon. After 20 min at this temperature, a precipate formed; 2-bromo-4-fluoro-trifluoroacetanilide (4.1 g) was added, followed by the alcohol obtained in Step B (3.0 g). The resulting reaction mixture was stirred at room temperature for 3 hours then the solvent was removed in vacuo. The residue was purified by silica gel chromatography (eluent ethyl acetate/cyclohexane 2:8) to afford 3-[2-[(2-bromo-4-fluoro-phenyl)-(2,2,2-trifluoro-acetyl)-amino]-eth-(E)-ylidene]-piperidine-1-carboxylic acid tert-butyl ester (4.7 g, 1:1 mixture of diastereoisomers), which was identified by its mass and NMR spectra. LCMS retention time (min) 2.38; MS (ES+) 395/397 (MW-BOC).

Step D: tributyltin-hydride (3.7 ml) in degassed toluene (100 ml) was added dropwise over 20 min to a solution of the product obtained in Step C (4.6 g) and 1,1′-azobis(cyclohexanecarbonitrile) (0.31 g) in degassed toluene (100 ml) at 100° C. under argon. The resulting solution was stirred at 100° C. for 1.5 hour then the solvent was removed in vacuo. The residue was dissolved in methanol (190 ml) and water (29 ml) and potassium carbonate (14 g) was added; the resulting mixture was vigorously stirred at room temperature for 2 hours, concentrated in vacuo, partitioned between ethyl acetate and water and the aqueous layer was extracted three times with ethyl acetate. The combined organic layers were dried over sodium sulfate and the solvent was removed in vacuo. The residue was purified by silica gel chromatography (eluent ethyl acetate/cyclohexane 2:8) to afford 3-(5-fluoro-2,3-dihydro-1H-indol-3-yl)-piperidine-I-carboxylic acid tert-butyl ester (2.8 g, 1:1 mixture of diastereois.omers), which was identified by its mass and N spectra. LCMS retention time (min) 1.87; MS(ES+) 221 (MH⁺-BOC), 265 (MH⁺-isoprene), 321 (MH⁺).

Step E: To a solution of the product obtained in Step D (2.8 g) in dichloromethane (20 ml) was added sodium bicarbonate (2.2 g) and 2-chloroisonicotinoyl chloride (50% w/w in toluene, 4.6 g) and the reaction mixture was stirred at room temperature for 5 hours. The reaction mixture was poured into water, extracted with dichloromethane, the organic layers were dried over sodium sulfate the concentrated in vacuo to afford crude 3-[1-(2-Chloro-pyridine-4-carbonyl)-5-fluoro-2,3-dihydro-1H-indol-3-yl]-piperidine-1-carboxylic acid tert-butyl ester as a 1:1 mixture of diastereosiomers (3.8 g), which was identified by its mass and NMR spectra. LCMS retention time (min) 2.25; MS(ES+) 360 (MH⁺-BOC), 404 (MH⁺-isoprene). 2.8 g of this crude material was purified by silica gel chromatography (ethyl acetate/cyclohexane 8:2) to afford a pure sample of each diastereoisomer (0.16 g for the least polar isomer A, 0.26 g of the most polar isomer B, 1.9 g of mixture fractions).

Step F: The product obtained in Step E (1:1 mixture of isomers, 0.3 g) in dichloromethane (5 ml) was treated with trifluoroacetic acid (0.5 ml) for 3.5 hours at room temperature. The solvent was removed in vacuo and the residue treated with 4 chlorocinnamyl chloride (0.28 g) and diisopropylethylamine (0.56 ml) in acetonitrile (5 ml) for 16 hours at room temperature. Removal of the solvent and silica gel chromatography (ethyl acetate) afforded the title product as a white solid (0.3 g, 1:1 mixture of diastereoisomers). LCMS retention time (min) 1.62; MS(ES+) 510 (MH⁺). The pure isomers A and B obtained in Step E (0.15 g and 0.2 g respectively) were treated under the same conditions to afford diastereoisomerically pure samples of the title product (0.1 g and 0.14 g respectively).

### EXAMPLE 5

This Example illustrates the pesticidal/insecticidal properties of compounds of formula (I).

Test against were performed as follows:

Spodoptera littoralis (Egyptian cotton leafworm)

Cotton leaf discs were placed on agar in a 24-well microtiter plate and sprayed with test solutions at an application rate of 200 ppm. After drying, the leaf discs were infested with 5 L₁ larvae. The samples were checked for mortality, repellent effect, feeding behaviour, and growth regulation 3 days after treatment (DAT). The following compounds gave at least 80% control of Spodoptera littoralis:

III-3, III-26, III-49, LXXI-3, LXXI-26, LXX1.29, LXXI-49, LXXI.348, LXXIII.26, LXXVI.26, CCLXVIII-1, CCLXVIII-2, CCLXVIII-3 and CCLXVIII-4 Heliothis virescens (Tobacco budworm):

Eggs (0-24 h old) were placed in 24-well microtiter plate on artificial diet and treated with test solutions at an application rate of 200 ppm by pipetting. After an incubation period of 4 days, samples were checked for egg mortality, larval mortality, and growth regulation. The following compounds gave at least 80% control of Heliothis virescen:

III-3, III-26, III-49, LXXI-3, LXXI-26, LXX1.29, LXXI-49, LXXI.348, LXXIII.26, LXXVI.26, CCLXIII-1, CCLXVII-2, CCLXVIII-3 and CCLXVIII-4 Plutella xylostella (Diamond back moth):

24-well microtiter plate (MTP) with artificial diet was treated with test solutions at an application rate of 18.2 ppm by pipetting. After drying, the MTP's were infested with larvae (L2)(10-15 per well). After an incubation period of 5 days, samples were checked for larval mortality, antifeedant and growth regulation. The following compounds gave at least 80% control of Plutella xylostella:

LXXI-3, LXXI-26, LXXI-49, LXXIII.26, CCLXVIII-1 and CCLXVIII-3.

Tetranychus urticae (Two-spotted spider mite):

Bean leaf discs on agar in 24-well microtiter plates wer sprayed with test solutions at an application rate of 200 ppm. After drying, the leaf discs are infested with mite populations of mixed ages. 8 days later, discs are checked for egg mortality, larval mortality, and adult mortality. The following compounds gave at least 80% control of Tetrainychus urticae:

LXXI-3, LXXI-26 and CCLXVIII-1

Aedes aegypti (Yellow fever mosquito):

10-15 Aedes larvae (L2) together with a nutrition mixture are placed in 96-well microtiter plates. Test solutions at an application rate of 2 ppm are pipetted into the wells. 2 days later, insects were checked for mortality and growth inhibition. The following compounds gave at least 80% control of Aedes aegypti

III-3, III-26, III-49, LXXI-26, LXXI-49, LXXIII.26 and LXXVI.26

1. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I): 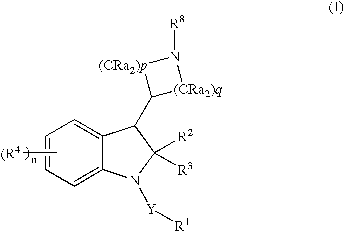
wherein Y is a single bond, C═O, C═S or S(O)m where m is 0, 1 or 2;
R¹ is hydrogen, optionally substituted alkyl, optionally substituted alkoxycarbonyl, optionally substituted alkylcarbonyl, aminocarbonyl, optionally substituted alkylaminocarbonyl, optionally substituted dialkylaminocarbonyl, optionally substituted aryl, optionally substituted heteroaryl, optionally substituted alkoxy, optionally substituted aryloxy, optionally substituted heteroaryloxy, optionally substituted heterocyclyloxy, cyano, optionally substituted alkenyl, optionally substituted alkynyl, optionally substituted cycloalkyl, optionally substituted cycloalkenyl, formyl, optionally substituted heterocyclyl, optionally substituted alkylthio, NO or NR¹³R¹⁴ where R¹³ and R¹⁴ are independently hydrogen, COR¹⁵, optionally substituted alkyl, optionally substituted aryl, optionally substituted heteroaryl, optionally substituted heterocyclyl or R¹³ and R¹⁴ together with the N atom to which they are attached form a group —N═C(R¹⁶)—NR¹⁷R¹⁸; R¹⁵ is H, optionally substituted alkyl, optionally substituted alkoxy, optionally substituted aryl, optionally substituted aryloxy optionally substituted heteroaryl, optionally substituted heteroaryloxy or NR¹⁹R²⁰; R¹⁶, R¹⁷ and R¹⁸ are each independently H or lower alkyl; R¹⁹ and R²⁰ are independently optionally substituted alkyl, optionally substituted aryl or optionally substituted heteroaryl;
R² and R³ are independently hydrogen, halogen, cyano, optionally substituted alkyl, optionally substituted alkoxy or optionally substituted aryl;
each R⁴ is independently halogen, nitro, cyano, optionally substituted C₁₋₈ alkyl, optionally substituted C₂₋₆ alkenyl, optionally substituted C₂₋₆ alkynyl, optionally substituted alkoxycarbonyl, optionally substituted alkylcarbonyl, optionally substituted alkylaminocarbonyl, optionally substituted dialkylaminocarbonyl, optionally substituted C₃₋₇ cycloalkyl, optionally substituted aryl, optionally substituted heteroaryl, optionally substituted heterocyclyl, optionally substituted alkoxy, optionally substituted aryloxy, optionally substituted heteroaryloxy, optionally substituted alkylthio or R²¹R²²N where R²¹ and R²² are, independently, hydrogen, C₁₋₈ alkyl, C₃₋₇ cycloalkyl, C₃₋₆ alkenyl, C₃₋₆ alkynyl, C₃₋₇ cycloalkyl(C₁₋₄)alkyl, C₂₋₆ haloalkyl, C₁₋₆ alkoxy(C₁₋₆)alkyl, C₁₋₆ alkoxycarbonyl or R²¹ and R²² together with the N atom to which they are attached form a five, six or seven-membered heterocyclic ring which may contain one or two further heteroatoms selected from O, N or S and which may be optionally substituted by one or two C₁₋₆ alkyl groups, or 2 adjacent groups R⁴ together with the carbon atoms to which they are attached form a 4, 5, 6,or 7 membered carbocyclic or heterocyclic ring which may be optionally substituted by halogen;
n is 0, 1, 2, 3 or 4;
each Ra is independently hydrogen, halogen, hydroxy, cyano, optionally substituted C₁₋₈ alkyl, optionally substituted C₂₋₆ alkenyl, optionally substituted C₂₋₆ alkynyl, optionally substituted alkoxycarbonyl, optionally substituted alkylcarbonyl, optionally substituted alkylaminocarbonyl, optionally substituted dialkylaminocarbonyl, optionally substituted C₃₋₇ cycloalkyl, optionally substituted aryl, optionally substituted heteroaryl, optionally substituted heterocyclyl, optionally substituted alkoxy, optionally substituted aryloxy, optionally substituted heteroaryloxy, optionally substituted alkylthio, optionally substituted arylthio or R²³R²⁴N where R²³ and R²⁴ are, independently, hydrogen, C₁₋₈ alkyl, C₃₋₇ cycloalkyl, C₃₋₆ alkenyl, C₃₋₆ alkynyl, C₃₋₇ cycloalkyl(C₁₋₄)alkyl, C₂₋₆ haloalkyl, C₁₋₆ alkoxy(C₁₋₆alkoxycarbonyl or R²³ and R²⁴ together with the N atom to which they are attached form a five, six or seven-membered heterocyclic ring which may contain one or two further heteroatoms selected from O, N or S and which may be optionally substituted by one or two C₁₋₆ alkyl groups, or two Ra groups attached to the same carbon atom are ═O or two Ra groups attached to adjacent carbon atoms form a bond, or two Ra groups together with the carbon atom to which they are attached form a three- to seven-membered ring, that may be saturated or unsaturated, and that may contain one or two hetero atoms selected from the group consisting of N, O and S, and which may be optionally substituted by one or two C₁₋₆ alkyl groups; or two Ra groups together form a group —CH₂—, —CH═CH— or —CH₂CH₂;p is 0, 1, 2, 3, 4, 5 or 6; q is 0, 1, 2, 3, 4, 5 or 6 provided that p+q is 1, 2, 3, 4, 5 or 6; R⁸ is optionally substituted alkyl, optionally substituted alkenyl, optionally substituted alkynyl, optionally substituted cycloalkyl, optionally substituted aryl, optionally substituted alkoxy, optionally substituted aryloxy, optionally substituted alkoxycarbonyl, optionally substituted alkylcarbonyl or optionally substituted alkenylcarbonyl; or salts or N-oxides thereof.

2. A method according to claim 1 wherein Y is a single bond or C═O.

3. A method according to claim 1 wherein R² and R³ are each independently hydrogen, C₁₋₆ alkyl, C₁₋₆ haloalkyl, C₁₋₆ alkoxy or cyano.

4. A method according to claim 1 wherein R¹ is hydrogen, C₁₋₆ alkyl, C₁₋₆ cyanoalkyl, C₁₋₆ haloalkyl, C₃₋₇ cycloalkyl(C₁₋₄)alkyl, C₁₋₆ alkoxy C₁₋₆)alkyl, heteroaryl(C₁₋₆)alkyl (wherein the heteroaryl group may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl C₁₋₆ alkylsulfinyl, C₁₋₆ alkylthio, C₁₋₆ alkoxycarbonyl, C₁₋₆ alkylcarbonylamino, arylcarbonyl, or two adjacent positions on the heteroaryl system may be cyclised to form a 5, 6 or 7 membered carbocyclic or heterocyclic ring, itself optionally substituted with halogen), aryl(C₁₋₆)alkyl (wherein the aryl group may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl, or two adjacent positions on the aryl system may be cyclised to form a 5, 6 or 7 membered carbocyclic or heterocyclic ring, itself optionally substituted with halogen), C₁₋₆ alkylcarbonylamino(C₁₋₆)alkyl, aryl (which may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl, or two adjacent positions on the aryl system may be cyclised to form a 5, 6 or 7 membered carbocyclic or heterocyclic ring, itself optionally substituted with halogen), heteroaryl (which may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl, or two adjacent positions on the heteroaryl system may be cyclised to form a 5, 6 or 7 membered carbocyclic or heterocyclic ring, itself optionally substituted with halogen), C₁₋₆ alkoxy, C₁₋₆ haloalkoxy, phenoxy (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryloxy (optionally substituted by halo, nitro, cyano, C₁₋₆ alkyl, C₁₋₆ haloalkyl, C₁₋₆ alkoxy or C₁₋₆ haloalkoxy), heterocyclyloxy (optionally substituted by halo, C₁₋₆ alkyl, C₁₋₆ haloalkyl, C₁₋₆ alkoxy or C₁₋₆ haloalkoxy), cyano, C₂₋₆ alkenyl, C₂₋₆ alkynyl, C₃₋₆ cycloalkyl, C₅₋₇ cycloalkenyl, heterocyclyl (optionally substituted by halo, nitro, cyano, C₁₋₆ alkyl, C₁₋₆ haloalkyl, C₁₋₆ alkoxy or C₁₋₆ haloalkoxy), Cl-6 alkylthio, C₁₋₆ haloalkylthio or NR¹³R¹⁴ where R¹³ and R¹⁴ are independently hydrogen, C₁₋₆ alkyl, C₁₋₆ haloalkyl, C₁₋₆ alkoxy(C₁₋₆)alkyl, phenyl (which may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino or C₁₋₄ alkoxycarbonyl), phenyl (C₁₋₆)alkyl (wherein the phenyl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl, or two adjacent positions on the phenyl ring may be cyclised to form a 5, 6 or 7 membered carbocyclic or heterocyclic ring, itself optionally substituted with halogen), heteroaryl (C₁₋₆)alkyl (wherein the heteroaryl group may be optionally substituted by halo, nitro, cyano, C₁₋₆ alkyl, Cl-6 haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl, or two adjacent positions on the heteroaryl system may be cyclised to form a 5, 6 or 7 membered carbocyclic or heterocyclic ring, itself optionally substituted with halogen) or heteroaryl (which may be optionally substituted by halo, nitro, cyano, C₁₋₆ alkyl, C₁₋₆ haloalkyl, C₁₋₆ alkoxy or C₁₋₆ haloalkoxy, C₁₋₄ alkoxycarbonyl C₁₋₆ alkylcarbonylamino, phenyloxycarbonylamino (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), amino, C016 alkylamino or phenylamino (wherein the phenyl group is optionally substituted halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino)).

5. A method according to claim 1 wherein each R⁴ is independently halogen, cyano, C₁₋₈ alkyl, C₁₋₈ haloalkyl, C₁₋₆ cyanoalkyl, C₁₋₆ alkoxy(C₁₋₆)alkyl, C₃₋₇ cycloalkyl(C₁₋₆)alkyl, C₅₋₆ cycloalkenyl(C₁₋₆)alkyl, C₃₋₆ alkenyloxy(C₁₋₆)alkyl, C₃₋₆ alkynyloxy(C₁₋₆)alkyl, aryloxy(C₁₋₆)alkyl, C₁₋₆ carboxyalkyl, C₁₋₆ alkylcarbonyl(C₁₋₆)alkyl, C₂₋₆ alkenylcarbonyl(C₁₋₆)alkyl, C₂₋₆ alkynylcarbonyl(C₁₋₆)-alkyl, C₁₋₆ alkoxycarbonyl(C₁₋₆)alkyl, C₃₋₆ alkenyloxycarbonyl(C₁₋₆)alkyl, C₃₋₆ alkynyloxycarbonyl(C₁₋₆)alkyl, aryloxycarbonyl(C₁₋₆)alkyl, C₁₋₆ alkylthio(C₁₋₆)alkyl, C₁₋₆ alkylsulfinyl(C₁₋₆)alkyl, C₁₋₆ alkylsulfonyl(C₁₋₆)alkyl, aminocarbonyl(C₁₋₆)alkyl, C₁₋₆alkylaminocarbonyl(C₁₋₆)alkyl, di(C₁₋₆)alkylaminocarbonyl(C₁₋₆)alkyl, phenyl(C₁₋₄)alkyl (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryl(C₁₋₄)alkyl (wherein the heteroaryl group is optionally substituted by halo, nitro, cyano, C₁₋₆ alkyl, C₁₋₆ haloalkyl, C₁₋₆ alkoxy or C₁₋₆ haloalkoxy), heterocyclyl(C₁₋₄)alkyl (wherein the heterocyclyl group is optionally substituted by halo, nitro, cyano, C₁₋₆ alkyl, C₁₋₆ haloalkyl, C₁₋₆ alkoxy or C₁₋₆ haloalkoxy), C₂₋₆ alkenyl, aminocarbonyl(C₂₋₆)alkenyl, C₁₋₆ alkylaminocarbonyl(C₂₋₆)alkenyl, di(C₁₋₆)alkylaminocarbonyl(C₂₋₆)alkenyl, phenyl(C₂₋₄)-alkenyl, (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), C₂₋₆ alkynyl, trimethylsilyl(C₂₋₆)alkynyl, aminocarbonyl(C₂₋₆)alkynyl, C₁₋₆ alkylaminocarbonyl(C₂₋₆)alkynyl, di(C₁₋₆)alkylaminocarbonyl(C₂₋₆)alkynyl, C₁₋₆ alkoxycarbonyl, C₃₋₇ cycloalkyl, C₃₋₇ halocycloalkyl, C₃₋₇ cyanocycloalkyl, C₁₋₃ alkyl(C₃₋₇)-cycloalkyl, C₁₋₃ alkyl(C₃₋₇)halocycloalkyl,phenyl (optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryl (optionally substituted by halo, nitro, cyano, C₁₋₆ alkyl, C₁₋₆ haloalkyl, C₁₋₆ alkoxy or C₁₋₆ haloalkoxy), heterocyclyl (wherein the heterocyclyl group is optionally substituted by halo, nitro, cyano, C₁₋₆ alkyl, C₁₋₆ haloalkyl, C₁₋₆ alkoxy or C₁₋₆ haloalkoxy), or 2 adjacent groups R⁴ together with the carbon atoms to which they are attached form a 4, 5, 6 or 7 membered carbocylic or heterocyclic ring which may be optionally substituted by halogen, C₁₋₈ alkoxy, C₁₋₆ haloalkoxy, phenoxy (optionally substituted by halo, nitro, cyano, C₁₋₆ alkyl, C₁₋₆ haloalkyl, C₁₋₆ alkoxy or C₁₋₆ haloalkoxy), heteroaryloxy (optionally substituted by halo, nitro, cyano, C₁₋₆ alkyl, C₁₋₆ haloalkyl, C₁₋₆ alkoxy or C₁₋₆ haloalkoxy), C₁₋₈ alkylthio or R¹⁹R²⁰N where R¹⁹ and R²⁰ are, independently, hydrogen, C₁₋₈ alkyl, C₃₋₇ cycloalkyl, C₃₋₆ alkenyl, C₃₋₆ alkynyl, C₂₋₆ haloalkyl, C₁₋₆ alkoxycarbonyl or R¹⁹ and R²⁰ together with the N atom to which they are attached form a five, six or seven-membered heterocyclic ring which may contain one or two further heteroatoms selected from O, N or S and which may be optionally substituted by one or two C₁₋₆ alkyl groups; and n is 0, 1, 2 or 3.

6. A method according to claim 1 wherein R⁸ is C₁₋₁₀ alkyl, C₁₋₁₀ haloalkyl, aryl(C₁₋₆)alkyl (wherein the aryl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryl(C₁₋₆)alkyl (wherein the heteroaryl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), arylcarbonyl-(C₁₋₆)alkyl (wherein the aryl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino and the alkyl group may be optionally substituted by aryl), C₂₋₈ alkenyl, C₂₋₈ haloalkenyl, aryl(C₂₋₆)-alkenyl (wherein the aryl group is optionally substituted halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino, C₁₋₆ alkoxycarbonyl, or two adjacent substituents can cyclise to form a 5, 6 or 7 membered carbocyclic or heterocyclic ring), heteroaryl(C₂₋₆)-alkenyl (wherein the heteroaryl group is optionally substituted halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino, C₁₋₆ alkoxycarbonyl, or two adjacent substituents can cyclise to form a 5, 6 or 7 membered carbocyclic or heterocyclic ring), C₂₋₆ alkynyl, phenyl(C₂₋₆)alkynyl (wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), C₃₋₇ cycloalkyl, C₁₋₆ alkoxycarbonyl, C₁₋₆ alkylcarbonyl, C₁₋₆ haloalkylcarbonyl or aryl(C₂₋₆)alkenylcarbonyl (wherein the aryl group may be optionally substituted halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), or —C(R⁵¹)(R⁵²)—[CR⁵³═CR⁵⁴]z-R⁵⁵ where z is 1 or 2, R⁵¹ and R⁵² are each independently H, halo or C₁₋₂ alkyl, R⁵³ and R⁵⁴ are each independently H, halogen, C₁₋₄alkyl or C₁₋₄haloalkyl and R⁵⁵ is optionally substituted aryl or optionally substituted heteroaryl.

7. A method according to claim 1 wherein each Ra is hydrogen.

8. A method according to claim 1 wherein p is 1 or 2 and q is 2.

9. A compound of formula I′ 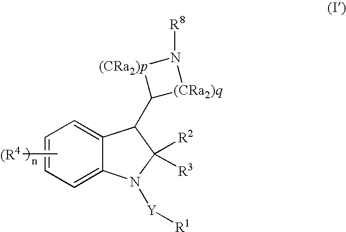
wherein Y is CO, R² and R³ are both hydrogen
R¹ is hydrogen, optionally substituted alkyl, optionally substituted alkoxycarbonyl, optionally substituted alkylcarbonyl, aminocarbonyl, optionally substituted alkylaminocarbonyl, optionally substituted dialkylaminocarbonyl, optionally substituted aryl, optionally substituted heteroaryl, optionally substituted alkoxy, optionally substituted aryloxy, optionally substituted heteroaryloxy, optionally substituted heterocyclyloxy, cyano, optionally substituted alkenyl, optionally substituted alkynyl, optionally substituted cycloalkyl, optionally substituted cycloalkenyl, formyl, optionally substituted heterocyclyl, optionally substituted alkylthio, NO or NR¹³R¹⁴ where R¹³ and R¹⁴ are independently hydrogen, COR¹⁵, optionally substituted alkyl, optionally substituted aryl, optionally substituted heteroaryl, optionally substituted heterocyclyl or R¹³ and R¹⁴ together with the N atom to which they are attached form a group —N═C(R¹⁶)—NR¹⁷R¹⁸; R¹⁵ is H, optionally substituted alkyl, optionally substituted alkoxy, optionally substituted aryl, optionally substituted aryloxy optionally substituted heteroaryl, optionally substituted heteroaryloxy or NR¹⁹R²⁰; R¹⁶, R¹⁷ and R¹³ are each independently H or lower alkyl; R¹⁹ and R²⁰ are independently optionally substituted alkyl, optionally substituted aryl or optionally substituted heteroaryl;
each R⁴ is independently halogen, nitro, cyano, optionally substituted C₁₋₈ alkyl, optionally substituted C₂₋₆ alkenyl, optionally substituted C₂₋₆ alkynyl, optionally substituted alkoxycarbonyl, optionally substituted alkylcarbonyl, optionally substituted alkylaminocarbonyl, optionally substituted dialkylaminocarbonyl, optionally substituted C₃₋₇ cycloalkyl, optionally substituted aryl, optionally substituted heteroaryl, optionally substituted heterocyclyl, optionally substituted alkoxy, optionally substituted aryloxy, optionally substituted heteroaryloxy, optionally substituted alkylthio or R²¹R²²N where R²¹ and R²² are, independently, hydrogen, C₁₋₈ alkyl, C₃₋₇ cycloalkyl, C₃₋₆ alkenyl, C₃₋₆ alkynyl, C₃₋₇ cycloalkyl(C₁₋₄)alkyl, C₂₋₆ haloalkyl, C₁₋₆ alkoxy(C₁₋₆)alkyl, C₁₋₆ alkoxycarbonyl or R²¹ and R²² together with the N atom to which they are attached form a five, six or seven-membered heterocyclic ring which may contain one or two further heteroatoms selected from O, N or S and which may be optionally substituted by one or two C₁₋₆ alkyl groups, or 2 adjacent groups R⁴ together with the carbon atoms to which they are attached form a 4, 5, 6,or 7 membered carbocyclic or heterocyclic ring which may be optionally substituted by halogen;
n is 0, 1, 2, 3 or 4;
each Ra is independently hydrogen, halogen, hydroxy, cyano, optionally substituted C₁₋₈ alkyl, optionally substituted C₂₋₆ alkenyl, optionally substituted C₂₋₆ alkynyl, optionally substituted alkoxycarbonyl, optionally substituted alkylcarbonyl, optionally substituted alkylaminocarbonyl, optionally substituted dialkylaminocarbonyl, optionally substituted C₃₋₇ cycloalkyl, optionally substituted aryl, optionally substituted heteroaryl, optionally substituted heterocyclyl, optionally substituted alkoxy, optionally substituted aryloxy, optionally substituted heteroaryloxy, optionally substituted alkylthio, optionally substituted arylthio or R²³R²⁴N where R²³ and R²⁴ are, independently, hydrogen, C₁₋₈ alkyl, C₃₋₇ cycloalkyl, C-₃₋₆ alkenyl, C₃₋₆ alkynyl, C₃₋₇ cycloalkyl(C₁₋₄)alkyl, C₂₋₆ haloalkyl, C₁₋₆ alkoxy(C₁₋₆) alkoxycarbonyl or R²³ and R²⁴ together with the N atom to which they are attached form a five, six or seven-membered heterocyclic ring which may contain one or two further heteroatoms selected from O, N or S and which may be optionally substituted by one or two C₁₋₆ alkyl groups, or two Ra groups attached to the same carbon atom are ═O or two Ra groups attached to adjacent carbon atoms form a bond, or two Ra groups together with the carbon atom to which they are attached form a three- to seven-membered ring, that may be saturated or unsaturated, and that may contain one or two hetero atoms selected from the group consisting of N, O and S, and which may be optionally substituted by one or two C₁₋₆ alkyl groups; or two Ra groups together form a group —CH₂—, —CH═CH— or —CH₂CH₂;
p is 0, 1, 2, 3, 4, 5 or 6; q is 0, 1, 2, 3, 4, 5 or 6 provided that p+q is 1, 2, 3, 4, 5 or 6;
R⁸ is optionally substituted alkyl, optionally substituted alkenyl, optionally substituted alkynyl, optionally substituted cycloalkyl, optionally substituted aryl, optionally substituted alkoxy, optionally substituted aryloxy, optionally substituted alkoxycarbonyl, optionally substituted alkylcarbonyl or optionally substituted alkenylcarbonyl; or salts or N-oxides thereof provided that when n is 0 and R¹ is CH₃ then the ring containing (CRa2)p and C(Ra2)q is not 1,2,3,6-tetrahydro-1,4-dimethyl-2-pyridinyl, 1,2,5,6-tetrahydro-1,4-dimethyl-2-pyridinyl or 2-pyrrolidinyl.

10. A compound of formula II 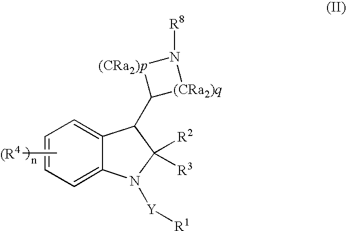
wherein Y, n, p, q, R¹, R², R³, R⁴ and Ra are as defined in claim 9 and R⁸ is hydrogen or tert-butoxycarbonyl.

11. An insecticidal acaricidal and nematicidal composition comprising an insecticidally, acaricidally or nematicidally effective amount of a compound of formula I as defined in claim 1.
